(19) United States
(12) Patent Application Publication
(10) Pub. No.: US 2021/0138066 A1
(43) Pub. Date: May 13, 2021

(54) REMOTE-CONTROLLED IMAGE-GUIDED DRUG DELIVERY VIA ULTRASOUND-MODULATED MOLECULAR DIFFUSION

(71) Applicant: California Institute of Technology, Pasadena, CA (US)

(72) Inventors: Maria Paulene B. Abundo, Pasadena, CA (US); Mikhail Shapiro, Los Angeles, CA (US)

(21) Appl. No.: 17/092,215

(22) Filed: Nov. 7, 2020

Related U.S. Application Data
(60) Provisional application No. 62/933,059, filed on Nov. 8, 2019.

(51) Int. Cl. A61K 41/00; A61N 7/00; A61K 47/46; A61K 47/32; A61K 47/36; A61K 49/00; A61K 47/42; C07K 16/24

(52) U.S. Cl. CPC A61K 41/0028; A61N 7/00; A61K 47/46; A61K 47/32; A61K 47/36; A61N 2007/0039; A61K 49/0032; A61K 47/42; C07K 16/241; A61K 49/0067; A61N 2007/0004; A61K 49/0054

(57) ABSTRACT

Disclosed herein include methods, compositions, and kits suitable for use in the spatial and temporal delivery of payload molecules to a target site of a subject. Disclosed herein include hydrogel compositions (e.g., particles) comprising a polymer scaffold, a plurality of payload molecules, and a plurality of gas vesicles. The method can comprise administering said hydrogel compositions to a subject and applying one or more ultrasonic (US) pulses to a target site of the subject to induce the release of payload molecules from the hydrogel composition, thereby delivering payload molecules to the target site. The method can comprise detecting the presence of the hydrogel composition at the target site prior to inducing release of payload molecules from the hydrogel composition.

### RELATED APPLICATIONS

This application claims the benefit under 35 U.S.C. § 119(e) of U.S. Provisional Patent Application Ser. No. 62/933,059, filed Nov. 8, 2019, the content of this related application is incorporated herein by reference in its entirety for all purposes.

### BACKGROUND

### Field

The present disclosure relates generally to the field of targeted payload delivery, and more particularly to compositions and methods for the spatially and temporally controlled delivery of payload molecules to target sites.

### Description of the Related Art

The development of technologies for remote-controlled, targeted drug delivery represents a “holy grail” of biomedical research. If implemented successfully, such platforms could enable the transport of potent small molecules, biologics or gene therapy treatments to specific anatomical locations in complex tissues such as the gastrointestinal (GI) tract, thereby maximizing efficacy at the intended site of action while minimizing side-effects. Ideally, remote-controlled drug delivery would be performed under image guidance, making it possible to monitor vehicle location, trigger release, and confirm payload delivery. These design features are important for therapeutic strategies to manage incurable inflammatory bowel diseases (IBD) such as Crohn's disease, which is notorious for its repeat episodes of discontinuous inflammation along extensive yet inaccessible areas of the gut.

Oral dosage forms that exploit regional variations in the GI tract such as pH, transit times and microbiome populations to delay or control the release of anti-inflammatory drugs are currently the cheapest, most common treatment for low-risk IBD symptoms. Despite the high patient compliance due to its convenience, these stimuli-based methods tend to be unreliable as basal GI tract physiology can be heavily altered by diet, disease, bowel movement and enterectomy procedures. For severe cases of IBD that necessitate greater specificity and drug bioavailability to diseased tissues, externally triggered electromechanical devices such as the capsule endoscope are preferred and administered clinically. However, these devices are expensive, challenging to produce, difficult to miniaturize and require sophisticated external apparatus for monitoring and control.

Hybrid polymeric drug delivery “soft devices” that can be externally triggered using radiation, ultrasound (“US”) and magnetic fields have later been developed as an alternative to electromechanical actuation by enhancing the capabilities of polymer-based formulations in performing spatiotemporal payload targeting. Amongst feasible external stimuli, US remains the most attractive option for triggering drug release as it is already widely used to noninvasively image whole organs at excellent spatial and temporal resolutions of around 0.1 to 1 mm and 1-100 ms respectively. Nevertheless, these soft devices were mostly designed for systemic administration and were often unsuitable for oral delivery as the diverse gastrointestinal physiology can interfere with the sensitive chemistries that trigger polymeric microstructural actuation. Besides the complex synthesis requirements, these systems also typically require the addition of contrast agents in order to locate the delivery vehicle in vivo, further complicating the drug formulation process.

In order for externally triggered soft devices to eventually be a viable method of treating patients, the drug release actuation and in vivo tracking mechanisms have to be greatly streamlined to enable its formulation into conventional oral dosage forms that can be self-administered and cheaply manufactured. There is a need for compositions, methods, and systems allowing the remote-controlled image-guided delivery of payloads to target sites.

### SUMMARY

Disclosed herein include hydrogel compositions. In some embodiments, the hydrogel composition comprises: a polymer scaffold comprising a plurality of polymers, a plurality of payload molecules, and a plurality of gas vesicles. As described in detail below, gas vesicles” can comprise protein-shelled nanostructures formed inside cells based on a specific genetic program, and can be purified from inside the cells. The phrases “gas vesicles protein structure” or “GV”, “GVP”, “GVPS” or “gas vesicles” as used herein shall be given their ordinary meaning, and shall also refer to a gas-filled protein structure intracellularly expressed by certain bacteria or archea as a mechanism to regulate cellular buoyancy in aqueous environments. In some embodiments, hydrogel compositions (e.g., hydrogels) and methods enabling control of hydrogel bulk properties using ultrasound via the introduction of acoustic nanoadditives. These additives can be based on gas vesicles (GVs), which are gas-filled protein nanostructures found in cyanobacteria whose native function is to regulate cellular buoyancy.

In some embodiments, the hydrogel composition comprises one or more particles. In some embodiments, the plurality of payload molecules and the plurality of gas vesicles are embedded within the polymer scaffold. In some embodiments, the plurality of polymers are cross-linked. In some embodiments, the plurality of polymers are cross-linked via (1) covalent bonds and/or non-covalent bonds, (2) inter-polymer bonds and/or intra-polymer bonds, and/or (3) physical crosslinks and/or chemical crosslinks. In some embodiments, the physical crosslinks are formed by a mechanism selected from the group consisting of ionic interactions, hydrophobic interactions, hydrogen bonding, Van der Waals forces and desolvation. In some embodiments, the chemical crosslinks are formed by a mechanism selected from the group consisting of free radical polymerization, condensation polymerization, anionic or cationic polymerization and step growth polymerization. In some embodiments, the plurality of payload molecules and/or the plurality of gas vesicles are not covalently bound to the polymer scaffold.

In some embodiments, the plurality of polymers comprise a copolymer. In some embodiments, the copolymer is a block copolymer. In some embodiments, the plurality of polymers comprise a natural polymer. In some embodiments, the natural polymer comprises alginate, agarose, carrageenan, chitosan, dextran, carboxymethylcellulose, heparin, hyaluronic acid, polyamino acid, collagen, gelatin, fibrin, a fibrous protein-based biopolymer, or any combination thereof. In some embodiments, the plurality of polymers comprise a synthetic polymer. In some embodiments, the synthetic polymer comprises alginic acid-polyethylene glycol copolymer, poly(ethylene glycol), poly(2-methyl-2-oxazoline), poly(ethylene oxide), poly(vinyl alcohol), poly(acrylamide), poly(n-butyl acrylate), poly-(α-esters), poly(glycolic acid), poly(lactic-co-glycolic acid), poly(L-lactic acid), poly(N-isopropylacrylamide), butyryl-trihexyl-citrate, di(2-ethylhexyl)phthalate, di-iso-nonyl-1,2-cyclohexanedicarboxylate, expanded polytetrafluoroethylene, ethylene vinyl alcohol copolymer, poly(hexamethylene diisocyanate), highly crosslinked poly(ethylene), poly(isophorone diisocyanate), poly(amide), poly(acrylonitrile), poly(carbonate), poly(caprolactone diol), poly(D-lactic acid), poly(dimethylsiloxane), poly(dioxanone), poly(ethylene), polyether ether ketone, polyester polymer alloy, polyether sulfone, poly(ethylene terephthalate), poly(hydroxyethyl methacrylate), poly(methyl methacrylate), poly(methylpentene), poly(propylene), polysulfone, poly(vinyl chloride), poly(vinylidene fluoride), poly(vinylpyrrolidone), poly(styrene-b-isobutylene-b-styrene), or any combination thereof. In some embodiments, the plurality of polymers comprise a polymer selected from the group comprising collagen, gelatin, chitosan, alginate, hyaluronic acid, dextran, polylactic acid, polyglycolic acid, poly(lactic acid-co-glycolic acid), polycaprolactone, polyanhydride, polyorthoester, polyvinyl alcohol, polyethylene glycol, polyurethane, polyacrylic acid, poly-N-isopropylacrylamide, poly(ethylene oxide)-poly(propylene oxide)-poly(ethylene oxide) copolymer, copolymers thereof, or any combination thereof. In some embodiments, the plurality of polymers comprise polystyrene, neoprene, polyetherether 10 ketone (PEEK), carbon reinforced PEEK, polyphenylene, PEKK, PAEK, polyphenylsulphone, polysulphone, PET, polyurethane, polyethylene, low-density polyethylene (LDPE), linear low-density polyethylene (LLDPE), high-density polyethylene (HDPE), polypropylene, polyetherketoneetherketoneketone (PEKEKK), nylon, TEFLON® TFE, polyethylene terephthalate (PETE), TEFLON® FEP, TEFLON® PFA, and/or polymethylpentene (PMP) styrene maleic anhydride, styrene maleic acid, polyurethane, silicone, polymethyl methacrylate, polyacrylonitrile, poly (carbonate-urethane), poly (vinylacetate), nitrocellulose, cellulose acetate, urethane, urethane/carbonate, polylactic acid, polyacrylamide (PAAM), poly (N-isopropylacrylamine) (PNIPAM), poly (vinylmethylether), poly (ethylene oxide), poly (ethyl (hydroxyethyl) cellulose), poly(2-ethyl oxazoline), polylactide (PLA), polyglycolide (PGA), poly(lactide-co-glycolide) PLGA, poly(e-caprolactone), polydiaoxanone, polyanhydride, trimethylene carbonate, poly(p-ydroxybutyrate), poly(g-ethyl glutamate), poly(DTH-iminocarbonate), poly(bisphenol A iminocarbonate), poly(orthoester) (POE), polycyanoacrylate (PCA), polyphosphazene, polyethyleneoxide (PEO), polyethylene glycol (PEG), polyacrylacid (PAA), polyacrylonitrile (PAN), polyvinylacrylate (PVA), polyvinylpyrrolidone (PVP), polyglycolic lactic acid (PGLA), poly(2-hydroxypropyl methacrylamide) (pHPMAm), poly(vinyl alcohol) (PVOH), PEG diacrylate (PEGDA), poly(hydroxyethyl methacrylate) (pHEMA), N-i sopropylacrylamide (NIPA), poly(vinyl alcohol) poly(acrylic acid) (PVOH-PAA), collagen, silk, fibrin, gelatin, hyaluron, cellulose, chitin, dextran, casein, albumin, ovalbumin, heparin sulfate, starch, agar, heparin, alginate, fibronectin, fibrin, keratin, pectin, elastin, ethylene vinyl acetate, polyethylene oxide, PEG or any of its derivatives, PLLA, PDMS, PIPA, PEVA, PILA, PEG styrene, Teflon RFE, FLPE, Teflon FEP, methyl palmitate, NIPA, polycarbonate, polyethersulfone, polycaprolactone, polymethyl methacrylate, polyisobutylene, nitrocellulose, medical grade silicone, cellulose acetate, cellulose acetate butyrate, polyacrylonitrile, PLCL, and/or chitosan.

In some embodiments, the hydrogel composition comprises an elastic modulus of less than about 100,000 pascals (Pa), of less than about 10,000 Pa, of less than about 1,000 Pa, or of less than about 100 Pa. In some embodiments, the polymer scaffold is biodegradable, biocompatible and/or hydrophilic. In some embodiments, the polymer scaffold is a water-insoluble network of polymers. In some embodiments, the hydrogel composition comprises a water content between about 10% and about 100%.

In some embodiments, the polymer scaffold comprises about 0.1% to about 50% volume per volume (% v/v), weight per volume (% w/v) or weight per weight (% w/w) of the hydrogel composition. In some embodiments, the plurality of gas vesicles comprise about 0.1% to about 50% volume per volume (% v/v), weight per volume (% w/v) or weight per weight (% w/w) of the hydrogel composition. In some embodiments, the plurality of payload molecules comprise about 0.1% to about 50% volume per volume (% v/v), weight per volume (% w/v) or weight per weight (% w/w) of the hydrogel composition. In some embodiments, less than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the plurality of payload molecules and/or the plurality of gas vesicles within the hydrogel composition are released from the hydrogel composition within a 24 hour period in the absence of exposure to ultrasound.

In some embodiments, the polymer scaffold comprises a plurality of pores. In some embodiments, the polymer scaffold comprises a porosity of at least about 10%, at least about 20%, at least about 30%, at least about 40%, at least about 50%, at least about 60%, at least about 70%, at least about 80%, or at least about 90%. In some embodiments, the polymer scaffold comprises an average pore size in the range of about 1 nm to about 30000 nm. In some embodiments, the polymer scaffold comprises an average pore size in the range of about 1 nm to about 100 nm. In some embodiments, the polymer scaffold comprises a pore size in the range of about 10 nm to about 1,000 μm and a porosity in the range of about 40 to about 97%. In some embodiments, the polymer scaffold does not comprise pores more than 150 nm in size. In some embodiments, the porosity of the polymer scaffold is configured to retain embedded payload molecules of less than about 100,000 kDa, of less than about 10,000 kDa, of less than about 1,000 kDa, of less than about 100 kDa, of less than about 100 kDa, or of less than about 10 kDa. In some embodiments, the porosity of the polymer scaffold is configured to retain embedded payload molecules with a hydrodynamic radius of less than about 100 nm, about 80 nm, about 60 nm, about 40 nm, about 30 nm, about 20 nm, about 10 nm, about 5 nm, or about 1 nm. In some embodiments, the average pore size of the polymer scaffold is smaller than the average diameter of the payload molecules.

In some embodiments, the one or more particles comprises an average diameter of between about 1 μm and about 1 cm. In some embodiments, the one or more particles comprises at least one cross-sectional dimension of between about 1 μm and about 1 cm. In some embodiments, the one or more particles have a substantially spheroid, cube, polyhedron, prism, cylinder, rod, and/or disc shape. In some embodiments, the one or more particles are substantially spherical and comprise a diameter of between about 1 μm and 1 cm. In some embodiments, a particle comprises an average diameter of less than about 1 m.

The hydrogel composition can comprise: a shell surrounding at least a portion of the polymer scaffold. In some embodiments, the shell comprises a thickness of from about 200 nm to about 200 μm, about 200 nm to about 750 nm, from about 200 nm to about 1 μm, from about 750 nm to about 50 μm, from about 1 μm to about 50 μm, from about 25 μm to about 50 μm, from about 2 μm to about 10 μm, or from about 2 μm to about 5 m. In some embodiments, the shell is biodegradable, biocompatible, and/or hydrophilic.

In some embodiments, the payload molecules comprise an immunosuppressant. In some embodiments, the plurality of payload molecules comprise a molecular weight of less than about 100,000 kDa, of less than about 10,000 kDa, of less than about 1,000 kDa, of less than about 100 kDa, of less than about 100 kDa, or of less than about 10 kDa. In some embodiments, the plurality of payload molecules comprise a hydrodynamic radius of about 1 nm to about 100 nm. In some embodiments, the plurality of payload molecules comprise a vector. In some embodiments, the vector is a viral vector. In some embodiments, the vector comprises an adenovirus vector, an adeno-associated virus vector, an Epstein-Barr virus vector, a Herpes virus vector, an attenuated HIV vector, a retroviral vector, a vaccinia virus vector, or any combination thereof. In some embodiments, the plurality of payload molecules comprise a contrast agent, an imaging agent, and/or a diagnostic agent.

In some embodiments, the plurality of payload molecules comprise a therapeutic agent useful for treating a disease of the GI tract. In some embodiments, the disease of the GI tract is an inflammatory bowel disease. In some embodiments, the therapeutic agent useful for treating inflammatory bowel disease comprises one of the following classes of compounds: 5-aminosalicyclic acids, corticosteroids, thiopurines, tumor necrosis factor-alpha blockers and JAK inhibitors. In some embodiments, the therapeutic agent useful for treating inflammatory bowel disease comprises one or more of Prednisone, Humira, Lialda, Imuran, Sulfasalazine, Pentasa, Mercaptopurine, Azathioprine, Apriso, Simponi, Enbrel, Humira Crohn's Disease Starter Pack, Colazal, Budesonide, Azulfidine, Purinethol, Proctoso! HC, Sulfazine EC, Delzicol, Balsalazide, Hydrocortisone acetate, Infliximab, Mesalamine, Proctozone-HC, Sulfazine, Orapred ODT, Mesalamine, Azasan, Asacol HD, Dipentum, Prednisone Intensol, Anusol-HC, Rowasa, Azulfidine EN-tabs, Veripred 20, Uceris, Adalimumab, Hydrocortisone, Colocort, Pediapred, Millipred, Azathioprine injection, Prednisolone sodium phosphate, Flo-Pred, Aminosalicylic acid, ProctoCream-HC, 5-aminosalicylic acid, Millipred DP, Golimumab, Prednisolone acetate, Rayos, Proctocort, Paser, Olsalazine, Procto-Pak, Purixan, Cortenema, Giazo, Vedolizumab, Entyvio, Micheliolide, and Parthenolide. In some embodiments, the plurality of payload molecules comprise a single dose.

In some embodiments, the plurality of payload molecules comprise a small molecule, a nucleic acid, a cell, a vector, a saccharine, a monosaccharide, a disaccharide, a trisaccharide, an oligosaccharide, a polysaccharide, a peptide, a protein, a peptide analog, a peptidomimetic, an antibody or antigen-binding fragment thereof, an antisense oligonucleotide, an siRNA, an shRNA, a ribozyme, an aptamer, a microRNA, a pre-microRNA, plasmid DNA, modified RNA, or any combination thereof. In some embodiments, the plurality of payload molecules treat and/or prevent a disease or disorder. In some embodiments, a payload molecule comprises comprise one or more of (i) a programmable nuclease or a nucleic acid encoding the programmable nuclease, (ii) a targeting molecule or a nucleic acid encoding the targeting molecule, and/or (iii) a donor nucleic acid or a nucleic acid encoding the donor nucleic acid. In some embodiments, the targeting molecule comprises a single guide RNA (sgRNA). In some embodiments, the programmable nuclease comprises a zinc finger nuclease, TAL effector nuclease, meganuclease, MegaTAL, Tev-m TALEN, MegaTev, homing endonuclease, derivatives thereof, or any combination thereof. In some embodiments, the programmable nuclease comprises Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof.

In some embodiments, the plurality of gas vesicles comprise an average cross-sectional diameter of about 40 nm to about 250 nm. In some embodiments, the plurality of gas vesicles comprise a gas permeable protein vesicle wall. In some embodiments, the plurality of gas vesicles are bacterially-derived gas vesicles and/or archaea-derived gas vesicles. In some embodiments, the plurality of gas vesicles are derived from a species of Anabaena bacteria, Halobacterium salinarum, and/or Bacillus megaterium. As provided herein, the plurality of gas vesicles are derived from said species by one or more methods, such as, for example, isolation from cultures of said species, genetic engineering of Gyp genes, heterologous expression of Gyp genes in other species, or any combination thereof. In some embodiments, the plurality of gas vesicles are capable of gas vesicle collapse. In some embodiments, at least one of the plurality of gas vesicle collapse comprises an at least about 10-fold reduction in volume. In some embodiments, at least one of the plurality of gas vesicle collapse comprises an at least about 10-fold reduction in acoustic contrast. In some embodiments, the plurality of gas vesicles are capable of acting as steric diffusion blockers to the payload molecules. In some embodiments, the gas vesicle collapse yields the formation of pores and/or percolation channels within the hydrogel composition. In some embodiments, the dimensions of said pores and/or percolation channels are larger than the dimensions of the payload molecules. In some embodiments, the gas vesicles are capable of impeding the diffusion of the payload molecules through the polymer scaffold. In some embodiments, the gas vesicle collapse increases the diffusivity of the hydrogel material by at least about 2-fold. In some embodiments, the gas vesicle collapse increases the porosity of the hydrogel composition by at least about 2-fold. In some embodiments, the gas vesicle collapse increases the rate of payload molecule release from the hydrogel composition by at least about 2-fold. In some embodiments, the percentage of noncollapsed gas vesicles within the hydrogel composition and diffusivity of the hydrogel composition are inversely related. In some embodiments, the gas vesicles reduce the diffusivity of the hydrogel composition by at least 2-fold as compared a hydrogel composition which does not comprise the gas vesicles.

In some embodiments, the plurality of gas vesicles comprise clustered gas vesicles. In some embodiments, the plurality of gas vesicles comprise a clustering moiety configured to form clustered gas vesicles. In some embodiments, the clustered gas vesicles comprise aggregates of two or more gas vesicles bound to each other via one or more clustering moieties. The clustering moiety can comprise biotin, avidin, streptavidin, heparin, an aptamer, a click-chemistry moiety, digoxigenin, anti-digoxigenin, dinitrophenol, anti-dinitrophenol. primary amine(s), carboxyl(s), hydroxyl(s), aldehyde(s), ketone(s), or any combination thereof. In some embodiments, the clustering of gas vesicles is achieved without the use of a cross-linker. For example, in some embodiments, clustering of gas vesicles can be accomplished via van der waals interactions, electrostatic interactions and/or depletion forces. In some embodiments, the clustered gas vesicles comprises at least one cross-sectional dimension of between about 100 nm and about 100 m. In some embodiments, the clustered gas vesicles comprise a fractal dimension between about 1 nm about 10 m. In some embodiments, the collapse of clustered gas vesicles leads to the formation of pores and/or percolation channels within the hydrogel composition that are at least 2-fold larger in at least one dimension as compared to the formation of pores and/or percolation channels within a hydrogel composition wherein the gas vesicles are not clustered.

The plurality of gas vesicles can comprise a first collapse pressure profile (e.g., peak acoustic pressure). In some embodiments, the first collapse pressure profile comprises a collapse function from which a gas vesicle collapse amount can be determined for a given pressure value. In some embodiments, the first collapse pressure profile comprises a first initial collapse pressure where 5% or lower of the plurality of gas vesicles are collapsed, a first midpoint collapse pressure where 50% of the plurality of gas vesicles are collapsed, a first complete collapse pressure where at least 95% of the plurality of gas vesicles are collapsed, any pressure between the first initial collapse pressure and the first midpoint collapse pressure, and any pressure between the first midpoint collapse pressure and the first complete collapse pressure. In some embodiments, a first selectable collapse pressure is any collapse pressure within the first collapse pressure profile. In some embodiments, a first selectable collapse pressure is selected from the first collapse pressure profile at a value between 0.05% collapse of the plurality of gas vesicles and 95% collapse of the plurality of gas vesicles. In some embodiments, a first selectable collapse pressure is equal to or greater than the first initial collapse pressure. In some embodiments, a first selectable collapse pressure is equal to or greater than the first midpoint collapse pressure. In some embodiments, a first selectable collapse pressure is equal to or greater than the first complete collapse pressure. As disclosed herein, the collapse pressure of gas vesicles can be tuned, by, for example, GypC-based tuning. GypC-based tuning of collapsed pressure can be employed in embodiments wherein a single GV type is employed. GypC-based tuning of collapsed pressure can also be employed in embodiments wherein a more than one GV type is employed. In some embodiments, the peak pressure is peak positive pressure. In some embodiments, the peak pressure is peak negative pressure. The collapse pressure profiles provided herein can relate to peak acoustic pressure and can be a function of the applied ultrasound frequency (e.g., increasing collapse pressure up to frequencies of 1 MHz and relatively steady above that). In some embodiments, the collapse pressure profiles provided herein do not relate to other pressure profiles (e.g., hydrostatic collapse pressure).

The hydrogel composition can comprise: a plurality of secondary gas vesicles. In some embodiments, the secondary gas vesicles comprise different mechanical, acoustic, and/or surface properties as compared to the gas vesicles. In some embodiments, the plurality of secondary gas vesicles comprises a second collapse pressure profile (e.g., peak acoustic pressure). In some embodiments, the second collapse pressure profile comprises a collapse function from which a secondary gas vesicle collapse amount can be determined for a given pressure value. In some embodiments, the first collapse pressure profile and the second collapse pressure profile are different. In some embodiments, the first collapse pressure profile and/or second collapse pressure profile has been configured by engineering a gas vesicle protein C (GvpC) protein of the gas vesicles and/or the secondary gas vesicles. In some embodiments, a midpoint of the second collapse profile has a higher pressure component than a midpoint of the first collapse profile. In some embodiments, the second collapse pressure profile comprises a second initial collapse pressure where 5% or lower of the plurality of secondary gas vesicles are collapsed, a second midpoint collapse pressure where 50% of the plurality of secondary gas vesicles are collapsed, a second complete collapse pressure where at least 95% of the plurality of secondary gas vesicles are collapsed, any pressure between the second initial collapse pressure and the second midpoint collapse pressure, and any pressure between the second midpoint collapse pressure and the second complete collapse pressure. In some embodiments, a second selectable collapse pressure is any collapse pressure within the second collapse pressure profile. In some embodiments, a second selectable collapse pressure is selected from the second collapse pressure profile at a value between 0.05% collapse of the plurality of secondary gas vesicles and 95% collapse of the plurality of secondary gas vesicles. In some embodiments, a second selectable collapse pressure is equal to or greater than the second initial collapse pressure. In some embodiments, a second selectable collapse pressure is equal to or greater than the second midpoint collapse pressure. In some embodiments, a second selectable collapse pressure is equal to or greater than the second complete collapse pressure. In some embodiments, the gas vesicles comprise one or more moieties configured to bind the polymer scaffold.

The hydrogel composition can comprise: one or more targeting moieties configured to bind a component of a target site of a subject. In some embodiments, the one or more targeting moieties comprise mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, and an RGD peptide or RGD peptide mimetic. In some embodiments, the one or more targeting moieties comprise one or more of the following: an antibody or antigen-binding fragment thereof, a peptide, a polypeptide, an enzyme, a peptidomimetic, a glycoprotein, a lectin, a nucleic acid, a monosaccharide, a disaccharide, a trisaccharide, an oligosaccharide, a polysaccharide, a glycosaminoglycan, a lipopolysaccharide, a lipid, a vitamin, a steroid, a hormone, a cofactor, a receptor, a receptor ligand, and analogs and derivatives thereof. In some embodiments, the antibody or antigen-binding fragment thereof comprises a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising complementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof. The one or more targeting moieties can be configured to bind one or more of the following: CD3, CD4, CD5, CD6, CD7, CD8, CD9, CD10, CD11a, CD11b, CD11c, CD12w, CD14, CD15, CD16, CDw7, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD46, CD47, CD48, CD49b, CD49c, CD51, CD52, CD53, CD54, CD55, CD56, CD58, CD59, CD61, CD62E, CD62L, CD62P, CD63, CD66, CD68, CD69, CD70, CD72, CD74, CD79, CD79a, CD79b, CD80, CD81, CD82, CD83, CD86, CD87, CD88, CD89, CD90, CD91, CD95, CD96, CD98, CD100, CD103, CD105, CD106, CD109, CD117, CD120, CD125, CD126, CD127, CD133, CD134, CD135, CD137, CD138, CD141, CD142, CD143, CD144, CD147, CD151, CD147, CD152, CD154, CD156, CD158, CD163, CD166, CD168, CD174, CD180, CD184, CDw186, CD194, CD195, CD200, CD200a, CD200b, CD209, CD221, CD227, CD235a, CD240, CD262, CD271, CD274, CD276 (B7-H3), CD303, CD304, CD309, CD326, 4-1BB, 5 AC, 5T4 (Trophoblast glycoprotein, TPBG, 5T4, Wnt-Activated Inhibitory Factor 1 or WAIF1), Adenocarcinoma antigen, AGS-5, AGS-22M6, Activin receptor like kinase 1, AFP, AKAP-4, ALK, Alpha integrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), B-lymphoma cell, bcr-abl, Bombesin, BORIS, C5, C242 antigen, CA125 (carbohydrate antigen 125, MUC16), CA-IX (CAIX, carbonic anhydrase 9), CALLA, CanAg, Canis lupus familiaris IL31, Carbonic anhydrase IX, Cardiac myosin, CCL11 (C-C motif chemokine 11), CCR4 (C-C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, CRIPTO, FCSF1R (Colony stimulating factor 1 receptor, CD 115), CSF2 (colony stimulating factor 2, Granulocyte-macrophage colony-stimulating factor (GM-CSF)), CTLA4 (cytotoxic T-lymphocyte-associated protein 4), CTAA16.88 tumor antigen, CXCR4 (CD 184), C—X—C chemokine receptor type 4, cyclic ADP ribose hydrolase, Cyclin B 1, CYP1B 1, Cytomegalovirus, Cytomegalovirus glycoprotein B, Dabigatran, DLL4 (delta-like-ligand 4), DPP4 (Dipeptidyl-peptidase 4), DR5 (Death receptor 5), E. coli Shiga toxin type-1, E. coli Shiga toxin type-2, ED-B, EGFL7 (EGF-like domain-containing protein 7), EGFR, EGFRII, EGFRvIII, Endoglin (CD 105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen l.F protein of respiratory syncytial virus, Frizzled receptor, Fucosyl GM1, GD2 ganglioside, G-28 (a cell surface antigen glycolipid), GD3 idiotype, GloboH, Glypican 3, N-glycolylneuraminic acid, GM3, GMCSF receptor a-chain, Growth differentiation factor 8, GP100, GPNMB (Transmembrane glycoprotein NMB), GUCY2C (Guanylate cyclase 2C, guanylyl cyclase C(GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-7, Influenza hemagglutinin, IgE, IgE Fc region, IGHE, IL-1, IL-2 receptor (interleukin 2 receptor), IL-4, IL-5, IL-6, IL-6R (interleukin 6 receptor), IL-9, IL-10, IL-12, IL-13, IL-17, IL-17A, IL-20, IL-22, IL-23, IL31RA, ILGF2 (Insulin-like growth factor 2), Integrins (α4, αᵤβ₃, αvβ3, αₐβ₇, α5β1, α6β4, α7β7, α11β3, α5β5, αvβ5), Interferon gamma-induced protein, ITGA2, ITGB2, KIR2D, LCK, Le, Legumain, Lewis-Y antigen, LFA-1 (Lymphocyte function-associated antigen 1, CD11a), LHRH, LINGO-1, Lipoteichoic acid, LIV1A, LMP2, LTA, MAD-CT-1, MAD-CT-2, MAGE-1, MAGE-2, MAGE-3, MAGE A1, MAGE A3, MAGE 4, MARTI, MCP-1, MIF (Macrophage migration inhibitory factor, or glycosylation inhibiting factor (GIF)), MS4A1 (membrane-spanning 4-domains subfamily A member 1), MSLN (mesothelin), MUC (Mucin 1, cell surface associated (MUC1) or polymorphic epithelial mucin (PEM)), MUC1-KLH, MUC16 (CA125), MCPI (monocyte chemotactic protein 1), MelanA/MARTI, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES 1, P21, p53 nonmutant, P97, Page4, PAP, Paratope of anti-(N-glycolylneuraminic acid), PAX3, PAX5, PCSK9, PDCD1 (PD-1, Programmed cell death protein 1, CD279), PDGF-Ra (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-α, TGF-β (Transforming growth factor beta), TGF-01, TGF-02 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TRP-2, Tyrosinase, VCAM-1 (CD 106), VEGF, VEGF-A, VEGF-2 (CD309), VEGFR-1, VEGFR2, or vimentin, WT1, XAGE 1, or cells expressing any insulin growth factor receptors, or any epidermal growth factor receptors.

The one or more particles can comprise a mixture of particles. The mixture of particles (e.g., hydrogels) can be heterogenous. The mixture of particles can comprise between about 1 and about 100 particles with different compositions (e.g., different polymer matrix, payload, GV, or any combination thereof). The mixture of particles can comprise one or more first particles and one or more second particles. There are provided, in some embodiments, hydrogel compositions comprising a mixture of particles comprising different types of payload molecules and/or gas vesicles. The polymer matrix of the first particles and second particles can be the same or different. The ratio of first particles to second particles can vary. The one or more particles can comprise one or more first particles and one or more second particles. The one or more first particles can comprise a plurality of payload molecules and a plurality of gas vesicles. The one or more second particles comprise a plurality of secondary payload molecules and a plurality of secondary gas vesicles. The gas vesicles and secondary gas vesicles can have different collapse pressure profiles. In some embodiments of the methods provided herein, the payload molecules of the one or more first particles are delivered to a first target site, and the secondary payload molecules of the one or more second particles are delivered to a second target site. The first target site and the second target site can be the same or different areas of the subject. In some embodiments, the payload of the first particles is released at the first target site and the payload of the second particles is not. In some embodiments, the payload of the second particles is released at the second target site and the payload of the first particles is not. In some embodiments, the payload of the first particles is released prior to the payload of the second particles.

The method can comprise applying collapsing ultrasound (e.g., second US pulse(s)) that causes the collapse of the gas vesicles within the one or more first particles (and thereby induce release of embedded payload molecules) but does not cause the collapse of gas vesicles (e.g., secondary gas vesicles) situated within the one or more second particles. The method can further comprise applying collapsing ultrasound (e.g., fifth US pulse(s)) that cause the collapse of the secondary gas vesicles within the one or more second particles (and thereby induce release of embedded secondary payload molecules). The method can comprise detecting the presence of the one or more first particles at a target site (e.g., a first target site) using the gas vesicles as contrast agents as disclosed herein (e.g., imaging ultrasound, detecting scattering of first US pulse(s) and/or third US pulse(s)). The method can comprise detecting the presence of the one or more second particles at a target site using the secondary gas vesicles as contrast agents as disclosed herein (e.g., imaging ultrasound, detecting scattering of fourth US pulse(s) and/or sixth US pulse(s)). The method can comprise detecting the presence of the one or more first particles at a target site (e.g., a second target site) in the subject using the gas vesicles as contrast agents as disclosed herein and/or detecting the presence of the one or more first particles at a target site in the subject using the secondary gas vesicles as contrast agents as disclosed herein. Confirming the delivery of payload molecules to a target site (e.g., a first target site) can comprise detecting a difference in the images derived from the first US pulse(s) and third US pulse(s). Confirming the delivery of secondary payload molecules to a target site (e.g., a second target site) can comprise detecting a difference in the images derived from the fourth US pulse(s) and sixth US pulse(s). In some embodiments, imaging US is not performed. In some such embodiments, only collapsing US is performed. For example, in some embodiments, collapsing US can be applied to a general region of anatomy. In some embodiments, the first US pulse(s) and the third US pulse(s) have the same parameters. In some embodiments, the fourth US pulse(s) and the sixth US pulse(s) have the same parameters.

Disclosed herein include methods for the spatial and temporal delivery of payload molecules to a target site of a subject. In some embodiments, the method comprises: administering a hydrogel composition disclosed herein to a subject; and applying one or more ultrasonic (US) pulses to a target site of the subject, wherein the one or more US pulses induce the release of payload molecules from the hydrogel composition, thereby delivering payload molecules to the target site. Disclosed herein include methods of treating a disease or disorder. In some embodiments, the method comprises: administering a hydrogel composition disclosed herein to a subject; and applying one or more ultrasonic (US) pulses to a target site of the subject, wherein the one or more US pulses induce the release of payload molecules from the hydrogel composition, thereby delivering payload molecules to the target site and treat the subject of the disease or disorder. Applying one or more US pulses can comprise applying second US pulse(s). In some embodiments, the second US pulse(s) induces the release of payload molecules from the hydrogel composition. The method can comprise detecting the presence of the hydrogel composition at the target site prior to inducing the release of payload molecules from the hydrogel composition. Applying one or more US pulses can comprise applying fifth US pulse(s). The method can comprise applying first US pulse(s), applying third US pulse(s), applying fourth US pulse(s), and/or applying sixth US pulse(s) to the target site.

Disclosed herein include methods for the spatial and temporal delivery of payload molecules to a target site of a subject. In some embodiments, the method comprises: administering a hydrogel composition disclosed herein to a subject; applying first ultrasonic (US) pulse(s) to a target site of the subject; detecting the presence of the hydrogel composition at the target site; and applying second US pulse(s) to the target site of the subject, wherein the second US pulse(s) induces the release of payload molecules from the hydrogel composition, thereby delivering payload molecules to the target site.

Disclosed herein include methods of treating a disease or disorder. In some embodiments, the method comprises: administering a hydrogel composition disclosed herein to a subject suffering from a disease or disorder; applying first ultrasonic (US) pulse(s) to a target site of the subject; detecting the presence of the hydrogel composition at the target site; and applying second US pulse(s) to the target site of the subject, wherein the second US pulse(s) induces the release of payload molecules from the hydrogel composition, thereby delivering payload molecules to the target site and treat the subject of the disease or disorder.

In some embodiments, detecting the presence of the hydrogel composition at the target site comprises detecting scattering of the first US pulse(s) by the gas vesicles. The method can comprise: confirming the delivery of payload molecules at the target site. In some embodiments, confirming the delivery of payload molecules comprises detecting reduced scattering of the second US pulse(s) by the gas vesicles. In some embodiments, the gas vesicles are capable of acting as a contrast agent at the first US pulse(s) but not at the second US pulse(s). In some embodiments, the first US pulse(s) comprises a pressure value less than the first selectable collapse pressure value. In some embodiments, the second US pulse(s) comprises a pressure value equal to or higher than the first selectable collapse pressure value. In some embodiments, the second US pulse(s) induces gas vesicle collapse. In some embodiments, the second US pulse(s) increases the diffusivity of the hydrogel material by at least about 2-fold. In some embodiments, the second US pulse(s) increases the porosity of the hydrogel composition by at least about 2-fold. In some embodiments, the second US pulse(s) increases the rate of payload molecule release from the hydrogel composition by at least about 2-fold. In some embodiments, the gas vesicles are capable of acting as a contrast agent at the first US pulse(s) but not at the second US pulse(s).

The method can comprise confirming the delivery of payload molecules at the target site. Confirming the delivery of payload molecules at the target site can comprise applying third US pulse(s) to the target site. Confirming the delivery of payload molecules can comprise detecting reduced scattering of the third US pulse(s) by the gas vesicles. The third US pulse(s) can comprise a pressure value less than the first selectable collapse pressure value, further optionally the target site is a first target site.

In some embodiments, the gas vesicle collapse results in the release of a nanoscale air bubble. In some embodiments, the released nanoscale air bubble undergoes cavitation and is converted into a micron-scale air bubble. In some embodiments, the second US pulse(s) is capable of inducing cavitation. In some embodiments, the cavitation comprises cavitation of the gas vesicles and/or bubbles created by gas vesicle collapse. In some embodiments, the gas vesicles are capable as acting as the nuclei for the formation and/or cavitation of bubbles. In some embodiments, the cavitation comprises stable cavitation. In some embodiments, the cavitation comprises inertial cavitation. In some embodiments, the cavitation triggers the degradation of the hydrogel composition. In some embodiments, the cavitation induces the release of payload molecules from the hydrogel composition. In some embodiments, the cavitation exerts mechanical forces and/or thermal forces on the hydrogel composition, thereby inducing the release of payload molecules. In some embodiments, the target site comprises target cells. In some embodiments, the cavitation exerts mechanical forces and/or thermal forces on target cells proximate to the hydrogel composition, thereby enhancing uptake of payload molecules by said target cells. In some embodiments, said mechanical forces and/or thermal forces reduce the membrane permeability of target cells proximate to the hydrogel composition. In some embodiments, the peak positive pressure of the second US pulse(s) is equal to or higher than an initial collapse pressure of the gas vesicles, thereby collapsing the gas vesicles. In some embodiments, the peak negative pressure of the second US pulse(s) is below the critical cavitation pressure of the gas vesicles.

In some embodiments, at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the plurality of payload molecules are released at the target site. In some embodiments, less than about 5%, about 10%, about 15%, about 20%, abou^{t} 25%, about 30%, about 35%, about 40%, about 45%, about 50%, about 55%, about 60%, about 65%, about 70%, about 75%, about 80%, about 85%, about 90%, about 95%, or about 100% of the plurality of payload molecules are released at a location other than the target site. In some embodiments, at least about 5%, about 10%, about 15%, about 20%, about 25%, about 30%, about 35%, about 40%, about 45%, about 50%, about 55%, about 60%, abou^{t} 65%, about 70%, about 75%, about 80%, about 85%, about 90%, about 95%, or about 100% of the plurality of payload molecules are released from the hydrogel composition within about 1 ns, about 10 ns, about 100 ns, about 1 ms, about 10 ms, about 100 ms, or about 1 s after the second US pulse(s). In some embodiments, at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the plurality of payload molecules are released from the hydrogel composition within about 1 nm, about 10 nm, about 100 nm, about 1 μm, about 10 μm, about 100 μm, about 1 mm, about 10 mm, or about 100 mm of the location of the hydrogel composition at the time of the second US pulse(s). In some embodiments, the ratio of the concentration of payload molecules at the subject's target site to the concentration of payload molecules in subject's blood, serum, or plasma is about 2:1 to about 3000:1, about 2:1 to about 2000:1, about 2:1 to about 1000:1, or about 2:1 to about 600:1.

In some embodiments, the second US pulse(s) comprises a pressure value is selected from the first collapse pressure profile (e.g., peak acoustic pressure) that optimally maximizes collapse of the first gas vesicles while minimizing collapse of the secondary gas vesicles. In some embodiments, the first US pulse(s) and the second US pulse(s) comprise a pressure value lower than the second selectable collapse pressure value. The method can comprise: applying fifth US pulse(s) to the same or different target site of the subject. In some embodiments, the fifth US pulse(s) comprises a pressure value equal to or higher than the second selectable collapse pressure value. In some embodiments, the fifth US pulse(s) induces secondary gas vesicle collapse. In some embodiments, the fifth US pulse(s) increases the diffusivity of the hydrogel material by at least about 2-fold. In some embodiments, the fifth US pulse(s) increases the porosity of the hydrogel composition by at least about 2-fold. The hydrogel composition can comprise: a plurality of secondary payload molecules. In some embodiments, a secondary payload molecule comprises a hydrodynamic radius at least 1.1-folder larger than a payload molecule. In some embodiments, the fifth US pulse(s) increases the rate of secondary payload molecule release from the hydrogel composition by at least about 2-fold. The method can comprise: confirming the delivery of the secondary payload molecules to the same or different target site of the subject. In some embodiments, confirming the delivery of the secondary payload comprises detecting reduced scattering of the fifth US pulse(s) by the secondary gas vesicles. In some embodiments, the secondary gas vesicles are capable of acting as a contrast agent at the fourth US pulse(s) and sixth US pulse(s) but not at the fifth US pulse(s). In some embodiments, less than about 10% of the secondary gas vesicles are not collapsed following the second US pulse(s). In some embodiments, applying US pulse(s) comprises applying focused US pulse(s). In some embodiments, applying US pulse(s) comprises applying ultrasound at a frequency of 100 kHz to 100 MHz. In some embodiments, applying US pulse(s) comprises applying ultrasound at a frequency of 0.2 to 1.5 MHz. In some embodiments, applying US pulse(s) comprises applying ultrasound having a mechanical index in a range between 0.2 and 0.6. In some embodiments, the first US pulse(s), second US pulse(s), and/or fifth US pulse(s) comprises a peak pressure (e.g., peak positive pressure) of about 40 kPa to about 800 kPa. In some embodiments, the first US pulse(s), second US pulse(s), and/or fifth US pulse(s) comprises a peak pressure (e.g., peak positive pressure) of about 70 kPa to about 150 kPa, and/or about 440 kPa to about 605 kPa. In some embodiments, the peak pressure is peak positive pressure. In some embodiments, the peak pressure is peak negative pressure.

The method can comprise applying fifth US pulse(s) to the same or different target site of the subject. The fifth US pulse(s) can comprise a pressure value equal to or higher than the second selectable collapse pressure value. The method can comprise detecting the presence of the hydrogel composition at the same or different target site prior to applying fifth US pulse(s). Detecting the presence of the hydrogel composition at the same or different target site can comprise applying fourth ultrasonic (US) pulse(s) to the same or different target site. Detecting the presence of the hydrogel composition at the same or different target site can comprise detecting scattering of the fourth US pulse(s) by the gas vesicles. The fourth US pulse(s) can comprise a pressure value less than the second selectable collapse pressure value.

The fifth US pulse(s) can induce secondary gas vesicle collapse. The fifth US pulse(s) can induce the release of secondary payload molecules from the hydrogel composition, thereby delivering secondary payload molecules to the same or different target site (e.g., a second target site).

The method can comprise confirming the delivery of the secondary payload molecules to the same or different target site of the subject (e.g., a second target site). Confirming the delivery of secondary payload molecules can comprise applying sixth US pulse(s) to the same or different target site. Confirming the delivery of secondary payload molecules can comprise detecting reduced scattering of the sixth US pulse(s) by the gas vesicles. The sixth US pulse(s) can comprise a pressure value less than the second selectable collapse pressure value.

In some embodiments, applying first US pulse(s) comprises applying a single first US pulse, applying second US pulse(s) comprises applying a single second US pulse, applying third US pulse(s) comprises applying a single third US pulse, applying fourth US pulse(s) comprises applying a single fourth US pulse, applying fifth US pulse(s) comprises applying a single fifth US pulse, and/or applying sixth US pulse(s) comprises applying a single sixth US pulse. In some embodiments, applying first US pulse(s) comprises applying a set of first US pulses, applying second US pulse(s) comprises applying a set of second US pulses, applying third US pulse(s) comprises applying a set of third US pulses, and/or applying fourth US pulse(s) comprises applying a set of fourth US pulses, applying fifth US pulse(s) comprises applying a set of fifth US pulses, and/or applying sixth US pulse(s) comprises applying a set of sixth US pulses. In some embodiments, first US pulse(s) comprises a set of first US pulses, second US pulse(s) comprises a set of second US pulses, third US pulse(s) comprises a set of third US pulses, fourth US pulse(s) comprises a set of fourth US pulses, fifth US pulse(s) comprises a set of fifth US pulses, and/or sixth US pulse(s) comprises a set of sixth US pulses. In some embodiments, first US pulse(s) comprises a single first US pulse, second US pulse(s) comprises a single second US pulse, third US pulse(s) comprises a single third US pulse, fourth US pulse(s) comprises a single fourth US pulse, fifth US pulse(s) comprises a single fifth US pulse, and/or sixth US pulse(s) comprises a single sixth US pulse. The peak acoustic pressure for a single US pulse or a set of US pulses can range from about 1 pascals to about 10 megapascals (and all combinations and subcombinations of ranges therein). In some embodiments, the pulse(s) comprise a pulse train, and can have a pulse duration ranging from about 10 microseconds to about 10 seconds (and all combinations and subcombinations of ranges therein). In some embodiments, applying pulse(s) comprises applying multiple pulses to ensure spatial coverage and/or complete GV collapse.

In some embodiments, the subject has a disease or disorder. In some embodiments, the subject is a mammal. In some embodiments, the subject is a marine mammal. In some embodiments, the subject has a disease of the GI tract. In some embodiments, the disease of the GI tract is an inflammatory bowel disease. In some embodiments, the inflammatory bowel disease comprises Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease or Behcet's disease. In some embodiments, the target site comprises a section or subsection of the GI tract. In some embodiments, the section or subsection of the GI tract can be the stomach, proximal duodenum, distal duodenum, proximal jejunum, distal jejunum, proximal ileum, distal ileum, proximal cecum, distal cecum, proximal ascending colon, distal ascending colon, proximal transverse colon, distal transverse colon, proximal descending colon and distal descending colon, or any combination thereof. In some embodiments, the target site comprises a site of disease or disorder or is proximate to a site of a disease or disorder. In some embodiments, the location of the one or more sites of a disease or disorder is predetermined. In some embodiments, the location of the one or more sites of a disease or disorder is determined during the method. The target site can comprises a tissue. In some embodiments, the tissue comprises adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue and/or fat tissue. In some embodiments, the tissue is inflamed tissue. In some embodiments, the tissue comprises (i) grade I, grade II, grade III or grade IV cancerous tissue; (ii) metastatic cancerous tissue; (iii) mixed grade cancerous tissue; (iv) a sub-grade cancerous tissue; (v) healthy or normal tissue; and/or (vi) cancerous or abnormal tissue. In some embodiments, upon administration, the hydrogel composition accumulates in vasculature of cancerous tissue.

In some embodiments, the payload comprises a contrast agent, an imaging agent and/or a diagnostic agent. The method can comprise: determining the presence and/or progression of a disease or disorder using the contrast agent, the imaging agent and/or the diagnostic agent.

In some embodiments, administering comprises aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, and/or intradermal injection, or any combination thereof. In some embodiments, the period of time between the administering and applying the first US pulse(s) is about 48 hours, about 44 hours, about 40 hours, about 35 hours, about 30 hours, about 25 hours, 20 hours, 15 hours, 10 hours, about 8 hours, about 8 hours, about 8 hours, about 7 hours, about 6 hours, about 5 hours, about 4 hours, about 3 hours, about 2 hours, about 1 hour, about 30 minutes, about 15 minutes, about 10 minutes, or about 5 minutes. In some embodiments, the hydrogel composition is formulated for oral administration. In some embodiments, the hydrogel composition comprises an enteric coating. In some embodiments, the enteric coating comprises one or more of acetyltributyl citrate, carbomers, cellulose acetate phthalate, cellulose acetate succinate, ethyl cellulose, guar gum, hypromellose acetate succinate, hypromellose phthalate, polymethacrylates, polyvinyl acetate phthalate, shellac, tributyl citrate, triethyl citrate, white wax, and zein. In some embodiments, the method does not comprise a pH-dependent payload release mechanism.

### BRIEF DESCRIPTION OF THE DRAWINGS

FIGS. 1A-1C depict several embodiments of the hydrogel compositions and methods provided herein. FIG. 1A depicts a non-limiting exemplary schematic of a gas vesicle highlighting its ability to exclude volume from surrounding water and solute. FIG. 1B depicts TEM images showing intact (left) and ultrasound-induced collapsed (right) gas vesicles. FIG. 1C depicts a non-limiting exemplary schematic illustration of GVs acting as steric diffusion blockers to internally embedded payload. Upon ultrasound application, GVs are shown to collapse and liberate excluded volume within the gel, triggering rapid outflow diffusion of payload.

FIGS. 2A-2D depict data related to BSA-AlexaFluor diffusivity through GV-gels. FIG. 2A depicts a non-limiting exemplary illustration of the experimental set-up (top) and a fluorescence image showing diffusion of BSA-AlexaFluor through a GV-gel with and without ultrasound exposure (bottom). FIG. 2B depicts characteristic time-series fluorescence intensity curves. FIG. 2C depicts switch-like diffusivity changes of BSA-AlexaFluor through GV-gels with and without ultrasound exposure for varying GV and polyacrylamide volume fractions (n=3). FIG. 2D depicts a 100-fold and 10-fold diffusivity switch observed for clustered and unclustered GVs-gels respectively (n=3). These gels contain 15% GVs and 10% Polyacrylamide by volume.

FIGS. 3A-3D depict data related to in vitro cargo release kinetics. FIG. 3A depicts a non-limiting exemplary schematic of the 3D-printed mould used to form milimeter-sized cubes. FIG. 3B depicts a non-limiting exemplary diagram of the experimental set-up. FIG. 3C depicts the release profiles of GV-gels with and without ultrasound exposure (n=3). FIG. 3D depicts the release profiles of clustered GV-gels with and without ultrasound exposure.

FIGS. 4A-4D depict data related to changes in the material diffusivity of hydrogel compositions disclosed when exposed to ultrasound. FIG. 4A depicts data related to ultrasound-modulated diffusivity changes with a BSA payload. FIG. 4B depicts a non-limiting exemplary schematic of preclustering GVs using a biotin-streptavidin reaction. FIG. 4C depicts data related to ultrasound-modulated diffusivity changes of clustered GVs embedded in a polyacrylamide hydrogel. FIG. 4D depicts data related to GV US-induced collapse triggering changes in material diffusivity with a variety of payloads and hydrogel systems across clinically-relevant length scales.

FIGS. 5A-5E depict data related to in vitro release of payload from GV-gel vehicles. FIG. 5A depicts a non-limiting schematic illustration of casting delivery vehicles into a plate containing 1% agarose. FIG. 5B depicts data related to GV-gels imaged and collapsed using the Verasonics L22 transducer operating at 18 MHz at 12 V and 20 V, respectively. FIG. 5C depicts imaging data related to GV collapse. FIG. 5D depicts a non-limiting schematic illustration of GV-gel tablets placed into a stirred vial filled with simulated stomach fluid. FIG. 5E depicts data related to drug release kinetics in simulated stomach fluid.

FIGS. 6A-6B depict data related to in vivo release of payload from GV-gel vehicles in mice. FIG. 6A depict representative pre and post-collapse x-AM images of the vehicles in vivo. FIG. 6B depict representative fluorescence images of the three different mice groups.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include hydrogel compositions. In some embodiments, the hydrogel composition comprises: a polymer scaffold comprising a plurality of polymers, a plurality of payload molecules, and a plurality of gas vesicles.

Disclosed herein include methods for the spatial and temporal delivery of payload molecules to a target site of a subject. In some embodiments, the method comprises: administering a hydrogel composition disclosed herein to a subject; applying first ultrasonic (US) pulse(s) to a target site of the subject; detecting the presence of the hydrogel composition at the target site; and applying second US pulse(s) to the target site of the subject, wherein the second US pulse(s) induces the release of payload molecules from the hydrogel composition, thereby delivering payload molecules to the target site.

Disclosed herein include methods of treating a disease or disorder. In some embodiments, the method comprises: administering a hydrogel composition disclosed herein to a subject suffering from a disease or disorder; applying first ultrasonic (US) pulse(s) to a target site of the subject; detecting the presence of the hydrogel composition at the target site; and applying second US pulse(s) to the target site of the subject, wherein the second US pulse(s) induces the release of payload molecules from the hydrogel composition, thereby delivering payload molecules to the target site and treat the subject of the disease or disorder.

Disclosed herein include methods for the spatial and temporal delivery of payload molecules to a target site of a subject. In some embodiments, the method comprises: administering a hydrogel composition disclosed herein to a subject; and applying one or more ultrasonic (US) pulses to a target site of the subject, wherein the one or more US pulses induce the release of payload molecules from the hydrogel composition, thereby delivering payload molecules to the target site. Disclosed herein include methods of treating a disease or disorder. In some embodiments, the method comprises: administering a hydrogel composition disclosed herein to a subject; and applying one or more ultrasonic (US) pulses to a target site of the subject, wherein the one or more US pulses induce the release of payload molecules from the hydrogel composition, thereby delivering payload molecules to the target site and treat the subject of the disease or disorder. Applying one or more US pulses can comprise applying second US pulse(s). In some embodiments, the second US pulse(s) induces the release of payload molecules from the hydrogel composition. The method can comprise detecting the presence of the hydrogel composition at the target site prior to inducing the release of payload molecules from the hydrogel composition. Applying one or more US pulses can comprise applying fifth US pulse(s). The method can comprise applying first US pulse(s), applying third US pulse(s), applying fourth US pulse(s), and/or applying sixth US pulse(s) to the target site.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

The term “contrast enhanced imaging” or “imaging”, as used herein indicates a visualization of a target site performed with the aid of a contrast agent administered to the target site to improve the visibility of structures or fluids by devices process and techniques suitable to provide a visual representation of a target site. Accordingly a contrast agent is a substance that enhances the contrast of structures or fluids within the target site, producing a higher contrast image for evaluation.

The term “ultrasound imaging” or ultrasound scanning” or “sonography” as used herein indicate imaging performed with techniques based on the application of ultrasound. Ultrasound can refer to sound with frequencies higher than the audible limits of human beings, typically over 20 kHz. Ultrasound devices typically can range up to the gigahertz range of frequencies, with most medical ultrasound devices operating in the 1 to 18 MHz range. The amplitude of the waves relates to the intensity of the ultrasound, which in turn relates to the pressure created by the ultrasound waves. Applying ultrasound can be accomplished, for example, by sending strong, short electrical pulses to a piezoelectric transducer directed at the target. Ultrasound can be applied as a continuous wave, or as wave pulses as will be understood by a skilled person.

Accordingly, the wording “ultrasound imaging” as used herein can refer to in particular to the use of high frequency sound waves, typically broadband waves in the megahertz range, to image structures in the body. The image can be up to 3D with ultrasound. In particular, ultrasound imaging typically involves the use of a small transducer (probe) transmitting high-frequency sound waves to a target site and collecting the sounds that bounce back from the target site to provide the collected sound to a computer using sound waves to create an image of the target site. Ultrasound imaging allows detection of the function of moving structures in real-time. Ultrasound imaging works on the principle that different structures/fluids in the target site will attenuate and return sound differently depending on their composition. A contrast agent sometimes used with ultrasound imaging are microbubbles created by an agitated saline solution, which works due to the drop in density at the interface between the gas in the bubbles and the surrounding fluid, which creates a strong ultrasound reflection. Ultrasound imaging can be performed with conventional ultrasound techniques and devices displaying 2D images as well as three-dimensional (3-D) ultrasound that formats the sound wave data into 3-D images. In addition to 3D ultrasound imaging, ultrasound imaging also encompasses Doppler ultrasound imaging, which uses the Doppler Effect to measure and visualize movement, such as blood flow rates. Types of Doppler imaging includes continuous wave Doppler, where a continuous sinusoidal wave is used; pulsed wave Doppler, which uses pulsed waves transmitted at a constant repetition frequency, and color flow imaging, which uses the phase shift between pulses to determine velocity information which is given a false color (such as red=flow towards viewer and blue=flow away from viewer) superimposed on a grey-scale anatomical image. Ultrasound imaging can use linear or non-linear propagation depending on the signal level. Harmonic and harmonic transient ultrasound response imaging can be used for increased axial resolution, as harmonic waves are generated from non-linear distortions of the acoustic signal as the ultrasound waves insonate tissues in the body. Other ultrasound techniques and devices suitable to image a target site using ultrasound would be understood by a skilled person.

The compositions, methods and systems described herein can be used with compositions, methods and systems (e.g., gas vesicle compositions and ultrasonic methods) previously described in U.S. Patent Application Publication Nos. 2014/0288411, 2014/0288421, 2018/0030501, 2018/0038922, 2019/0175763, 2019/0314001, 2020/0164095, 2020/0237346, and International Patent Application Publication WO2020/146379; the content of each of these applications is incorporated herein by reference in its entirety.

Hydrogels

The ability to spatially and temporally control the porosity and diffusivity of hydrogels is a long-standing need in multiple areas of regenerative medicine. In some embodiments, hydrogel compositions (e.g., hydrogels) and methods enabling control of hydrogel bulk properties using ultrasound via the introduction of acoustic nanoadditives. These additives can be based on gas vesicles (GVs), which are gas-filled protein nanostructures found in cyanobacteria whose native function is to regulate cellular buoyancy. GVs can undergo pressure induced collapse, instantaneously reducing their volume by nearly 10-fold. Due to their rapid and drastic volume reduction in response to pressure, it is demonstrated herein that GVs can serve as nanoadditives in materials to influence bulk properties in a stimulus-responsive manner. As shown herein, GVs can act as stable inclusions when embedded in polyacrylamide hydrogels, without compromising gel integrity. Moreover, as disclosed herein, GVs can be acoustically collapsed by ultrasound in situ, forming nanoscale pores within the polymeric network. Additionally, disclosed herein is evidence that GVs, in their intact form, can impede the diffusion of proteins through the hydrogel, and upon in situ collapse, increase the diffusivity of the hydrogel material. As described herein, GVs are capable of serving as nanoadditives to enable acoustically responsive polymeric materials with locally tunable porosity and diffusivity, and thereby enables the disclosed applications in drug delivery and tissue engineering in a broad range of in vitro and in vivo contexts. In some embodiments, compositions and methods for remote-controlled image-guided oral delivery of payload to the gut with high spatiotemporal specificity using a generic hydrogel formulation. As described herein, hydrogel can be a network of crosslinked polymer chains that are hydrophilic, for example a colloidal gel in which water is the dispersion medium.

The hydrogel compositions (e.g., GV-gel, GV-gel vehicles) provided herein can serve as a versatile drug delivery platform designed to impart acoustic responsiveness and ultrasound contrast to pharmaceutically acceptable compositions (e.g., generic oral drug formulations) for use in methods a wide range of in vitro and in vivo contexts, such as, for example in the treatment of IBD. The compositions, methods, and system provided herein can combine the manufacturing ease and convenience of polymer-based formulations whilst enabling features typically found only in electromechanical devices such as the image-guided triggered release of payloads with high spatiotemporal resolution.

The unique applications of the hydrogel compositions provided herein are possible by exploiting GVs, a unique class of proteins derived from buoyant microbes, which possess 2-nm thick protein shells enclosing air-filled compartments with dimensions on the order of 200 nm. These hollow protein nanostructures are stable at physiologically relevant conditions by allowing air to partition across the shell interface whilst preventing the transport of water and solute particles (FIG. 1A). In its intact state, GVs effectively scatter sound waves and serve as ultrasound contrast agents that are currently being utilized in many in vivo applications to control and image biological function. These protein nanostructures can also be collapsed when exposed to ultrasound waves with amplitudes above a critical pressure threshold, resulting in the simultaneous loss of acoustic contrast and 99.9% of its volume (FIG. 1B).

When embedded in a drug-loaded polymer system, GVs can concurrently function as ultrasound imaging agents and steric-blockers that hinder diffusion of the drug out of the polymeric delivery vehicle, as well as the diffusion of any degrading factors into the GV (FIG. 1C). To trigger rapid drug release, the dispersed GVs can be collapsed to trigger the formation of percolation channels within the hydrogel material (FIG. 1C). Because ultrasound at pressures below the critical collapse threshold can be used to locate GVs, widely available ultrasound imaging systems can be used to track these GV-containing drug delivery vehicles inside the body, trigger payload release with high-amplitude imaging pulses, and confirm that GV collapse has taken place.

Disclosed herein include hydrogel compositions. In some embodiments, the hydrogel composition comprises: a polymer scaffold comprising a plurality of polymers, a plurality of payload molecules, and a plurality of gas vesicles. The plurality of payload molecules and the plurality of gas vesicles can be embedded within the polymer scaffold. The hydrogel composition can comprise one or more (e.g., 1, 2, 3, 4, 5, 6, 7, 8, 9, 10, 20, 30, 40, 50, 60, 70, 80, 90, 100, 200, 300, 400, 500, 600, 700, 800, 900, 1000, 10000, 100000, 1000000, 1000000, 1000000 particles, or a number or a range between any two of these values) particles. The one or more particles can comprise an average diameter of between about 1 nm and about 1 cm (e.g., about 1 nm, 5 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm, 10 μm, 20 μm, 30 m, 70 μm, 100 μm, 200 μm, 400 μm, 600 μm, 800 μm, 1000 μm, 2000 μm, 3000 am, 4000 am, 5000 μm, 6000 μm, 7000 μm, 8000 μm, 9000 μm, 10000 μm, or a number or a range between any of these values). The one or more particles can comprise at least one cross-sectional dimension of between about 1 nm and about 1 cm (e.g., about 1 nm, 5 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 m, 8 am, 9 μm, 10 μm, 20 μm, 30 μm, 70 μm, 100 am, 200 am, 400 am, 600 am, 800 am, 1000 am, 2000 am, 3000 am, 4000 am, 5000 am, 6000 am, 7000 am, 8000 am, 9000 am, 10000 m, or a number or a range between any of these values). In some embodiments, the one or more particles have a substantially spheroid, cube, polyhedron, prism, tube, sheet, cylinder, rod, and/or disc shape. The one or more particles can be substantially spherical and can comprise a diameter of between about 1 nm and 100 μm (e.g., about 1 nm, 5 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 m, 9 μm, 10 μm, 20 μm, 30 μm, 70 μm, 100 μm, or a number or a range between any of these values). In some embodiments, applications involving delivery of payload molecules to the GI tract comprise particles with a mm dimension. In some embodiments, applications involving delivery of payload molecules to humans comprise particles with a cm dimension.

In some embodiments, hydrogel compositions (e.g., hydrogels). The hydrogel composition can comprise a hydrogel. The hydrogel composition can comprise a polymer scaffold. A hydrogel can comprise a polymer scaffold. The hydrogel composition can comprise one or more additional components (e.g., gas-filled protein structures, payload molecules). The one or more additional components (e.g., gas-filled protein structures, payload molecules) can be embedded into the hydrogel. The term “hydrogel,” as used herein, shall be given its ordinary meaning, and shall also refer to a gel in which water is the dispersion medium. Typically, a hydrogel comprises a plurality of polymer molecules that are cross-linked, either via covalent bonds or via non-covalent interactions, thus forming a polymer scaffold, also referred to herein as a hydrogel scaffold. A hydrogel can refer to a water-swellable polymeric matrix formed from a three-dimensional network of macromolecules held together by covalent or non-covalent crosslinks, that can absorb a substantial amount of water (by weight) to form a gel. A hydrogel can refer to a network of polymer chains in an aqueous dispersion medium. In some preferred embodiments, the cross-linking is via covalent bonds. Cross-linking typically comprises inter-polymer bonds (bonds between different polymer molecules), but may also comprise intra-polymer bonds (bonds within the same polymer molecule). In some embodiments, the polymers are water-soluble in their non-cross-linked form, but are insoluble once they are cross-linked. Hydrogels useful in the context of this disclosure typically comprise a water content within the range of about 85% to about 99%. For example, in some embodiments, a hydrogel has a water content of about 99%, about 98%, about 97.5%, about 97%, about 96%, about 94%, about 93%, about 92%, about 91%, or about 90%. In some embodiments, hydrogels with a water content of less than 90% are employed. A hydrogel may comprise components in addition to the scaffold and water, for example, GVs and payload. The terms “hydrogel scaffold,” or “polymer scaffold” as used herein, can be used interchangeably, and can refer to a water-insoluble network of polymers within a hydrogel.

The hydrogel composition can comprise a shell surrounding at least a portion of the polymer scaffold. The shell can comprise a thickness of about 5 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 jm, 9 μm, 10 μm, 20 μm, 30 μm, 40 μm, 50 μm, 60 μm, 70 μm, 80 μm, 90 μm, 100 μm, 200 m, 300 μm, 400 μm, 500 μm, 600 μm, 700 μm, 800 μm, 900 μm, 1 mm, 2 mm, 3 mm, 4 mm, 5 mm, 6 mm, 7 mm, 8 mm, 9 mm, 1 cm, 2 cm, 3 cm, 4 cm, 5 cm, 6 cm, 7 cm, 8 cm, 9 cm, 10 cm, or a number or a range between any of these values. The shell can be biodegradable, biocompatible, and/or hydrophilic.

Cross-Linking

The hydrogels provided herein can comprise hydrogel scaffolds made of cross-linked polymers. The term “cross-linked,” as used herein, can refer to a type of binding involving a plurality of polymers and a plurality of binding interactions. Cross-linked polymers are polymers that are connected to form a network, and, in the context of hydrogels, a hydrogel scaffold. Accordingly, a polymer in a cross-linked state is connected to another polymer or a plurality of other polymers through two or more covalent bonds or non-covalent interactions, thus forming a network of interconnected polymer molecules. Cross-linking can be either via covalent bonds or via non-covalent interactions. In some embodiments, hydrogel-forming polysaccharides cross-link via non-covalent bonds, e.g., as is the case for alginates, via chelation of ions. In other embodiments, however, the polymers forming the hydrogel scaffold of an engineered hydrogel provided herein are cross-linked via covalent bonds. The formation of such covalently cross-linked hydrogel scaffolds can involve the formation of covalent bonds between individual polymer molecules, but may also involve the formation of intra-molecular bonds within the same polymer molecule.

In some embodiments, covalent bond-formation between hydrogel-forming polymer molecules involves a chemical reaction between reactive moieties comprised in or conjugated to the hydrogel-forming polymers. The term “reactive moiety,” as used herein in the context of hydrogel-forming polymers, can refer to a moiety comprised in or conjugated to a first polymer that can react with a second reactive moiety comprised in or conjugated to a second polymer to form a covalent bond. In some embodiments, a hydrogel-forming polymer comprises or is conjugated to a plurality of reactive moieties, which allows for the generation of covalent cross-links with a number of polymer molecules. In some embodiments the plurality of reactive moieties comprised in or conjugated to a polymer are of the same type. In other embodiments, a polymer may comprise or be conjugated to a plurality of different reactive moieties.

The plurality of polymers can be cross-linked. For example, the plurality of polymers can be cross-linked via (1) covalent bonds and/or non-covalent bonds, (2) inter-polymer bonds and/or intra-polymer bonds, and/or (3) physical crosslinks and/or chemical crosslinks. The physical crosslinks can be formed by a mechanism selected from the group consisting of ionic interactions, hydrophobic interactions, hydrogen bonding, Van der Waals forces and desolvation. The chemical crosslinks can be formed by a mechanism selected from the group consisting of free radical polymerization, condensation polymerization, anionic or cationic polymerization and step growth polymerization. In some embodiments, the plurality of payload molecules and/or the plurality of gas vesicles are not covalently Bound to the Polymer Scaffold.

Hydrogel-Forming Polymers

The term “polymer,” as used herein, can refer to a molecule comprising a plurality of repeating structural units (monomers), typically at least 3, linked together via covalent bonds. Non-limiting examples of polymers are polysaccharides, polynucleotides, and polypeptides. Exemplary hydrogel-forming polymers, e.g., dextrose, carboxymethylcellulose, and hyaluronic acid, are described in more detail elsewhere herein. Additional polymers that can form hydrogels are also encompassed. In some embodiments, where a hydrogel or hydrogel-forming composition is administered to a subject, the polymer and the respective hydrogel scaffold formed are preferably biocompatible in that they do not elicit an immune or inflammatory response once administered and in that the formation of the hydrogel scaffold does not result in toxic or otherwise harmful side reactions or side products.

It will be apparent to the skilled artisan that any suitable hydrogel scaffold can be employed in some embodiments of this disclosure, and that the exemplary scaffolds and hydrogel-forming polymers described herein in more detail are not in any way limiting. For example, in some embodiments, engineered hydrogels are provided that comprise polymer scaffolds made of polymers that are known in the art to be useful in the preparation of hydrogels. Such polymers may include, in some embodiments, e.g., cellulose derivatives, xyloglucans, chitosans, glycerophosphates, alginates, gelatin, polyethylene glycol, N-isopropylamide copolymers (e.g., poly(N-isopropylacrylamide-co-acrylic acid) or poly(N-isopropylacrylamide)/poly(ethylene oxide)), poloxamers (e.g., pluronic-modified poloxamer or poloxamer/poly(acrylicacid)), poly(ethylene oxide)/poly(D,L-lactic acid-co-glycolic acid), poly(organophosphazene), or poly(1,2-propylene phosphate), and their derivatives. Additional polymers useful for the formation of a hydrogel scaffold in the context of some embodiments of this disclosure will be apparent to those of skill in the art, and the disclosure is not limited in this respect.

The plurality of polymers can comprise a copolymer. The copolymer can be a block copolymer. The plurality of polymers can comprise a natural polymer (e.g., alginate, agarose, carrageenan, chitosan, dextran, carboxymethylcellulose, heparin, hyaluronic acid, polyamino acid, collagen, gelatin, fibrin, a fibrous protein-based biopolymer, or any combination thereof). A “natural polymer” can refer to a polymeric material that may be found in nature. In various embodiments, hydrogels are formed by natural polymers selected from the group consisting of polysaccharides, glycosaminoglycans, proteins, and mixtures thereof. These hydrogels may also be termed herein as “natural hydrogels”. Polysaccharides are carbohydrates which may be hydrolyzed to two or more monosaccharide molecules. They may contain a backbone of repeating carbohydrate i.e. sugar unit. Examples of polysaccharides include, but are not limited to, alginate, agarose, chitosan, dextran, starch, and gellan gum. Glycosaminoglycans are polysaccharides containing amino sugars as a component. Examples of glycosaminoglycans include, but are not limited to, hyaluronic acid, chondroitin sulfate, dermatin sulfate, keratin sulfate, dextran sulfate, heparin sulfate, heparin, glucuronic acid, iduronic acid, galactose, galactosamine, and glucosamine.

Examples of natural hydrogels which are well known in the art include alginate and agarose. In some embodiments, the degradable hydrogel comprises alginate. The term “alginate” can refer to any of the conventional salts of algin, which is a polysaccharide of marine algae, and which may be polymerized to form a matrix for use in drug delivery and in tissue engineering due to its biocompatibility, low toxicity, relatively low cost, and simple gelation with divalent cations such as calcium ions (Ca²⁺) and magnesium ions (Mg²⁺). Examples of alginate include sodium alginate which is water soluble, and calcium alginate which is insoluble in water. In some embodiments, agarose may be used as the hydrogel. Agarose can refer to a neutral gelling fraction of a polysaccharide complex extracted from the agarocytes of algae such as a Rhodophyceae. However, unlike alginate, it forms thermally reversible gels.

The plurality of polymers can comprise a synthetic polymer, such as, for example, alginic acid-polyethylene glycol copolymer, poly(ethylene glycol), poly(2-methyl-2-oxazoline), poly(ethylene oxide), poly(vinyl alcohol), poly(acrylamide), poly(n-butyl acrylate), poly-(α-esters), poly(glycolic acid), poly(lactic-co-glycolic acid), poly(L-lactic acid), poly(N-isopropylacrylamide), butyryl-trihexyl-citrate, di(2-ethylhexyl)phthalate, di-iso-nonyl-1,2-cyclohexanedicarboxylate, expanded polytetrafluoroethylene, ethylene vinyl alcohol copolymer, poly(hexamethylene diisocyanate), highly crosslinked poly(ethylene), poly(isophorone diisocyanate), poly(amide), poly(acrylonitrile), poly(carbonate), poly(caprolactone diol), poly(D-lactic acid), poly(dimethylsiloxane), poly(dioxanone), poly(ethylene), polyether ether ketone, polyester polymer alloy, polyether sulfone, poly(ethylene terephthalate), poly(hydroxyethyl methacrylate), poly(methyl methacrylate), poly(methylpentene), poly(propylene), polysulfone, poly(vinyl chloride), poly(vinylidene fluoride), poly(vinylpyrrolidone), poly(styrene-b-isobutylene-b-styrene), or any combination thereof.

The plurality of polymers can comprise a polymer selected from the group comprising collagen, gelatin, chitosan, alginate, hyaluronic acid, dextran, polylactic acid, polyglycolic acid, poly(lactic acid-co-glycolic acid), polycaprolactone, polyanhydride, polyorthoester, polyvinyl alcohol, polyethylene glycol, polyurethane, polyacrylic acid, poly-N-isopropylacrylamide, poly(ethylene oxide)-poly(propylene oxide)-poly(ethylene oxide) copolymer, copolymers thereof, or any combination thereof.

The plurality of polymers can comprise polystyrene, neoprene, polyetherether 10 ketone (PEEK), carbon reinforced PEEK, polyphenylene, PEKK, PAEK, polyphenylsulphone, polysulphone, PET, polyurethane, polyethylene, low-density polyethylene (LDPE), linear low-density polyethylene (LLDPE), high-density polyethylene (HDPE), polypropylene, polyetherketoneetherketoneketone (PEKEKK), nylon, TEFLON® TFE, polyethylene terephthalate (PETE), TEFLON® FEP, TEFLON® PFA, and/or polymethylpentene (PMP) styrene maleic anhydride, styrene maleic acid, polyurethane, silicone, polymethyl methacrylate, polyacrylonitrile, poly (carbonate-urethane), poly (vinylacetate), nitrocellulose, cellulose acetate, urethane, urethane/carbonate, polylactic acid, polyacrylamide (PAAM), poly (N-isopropylacrylamine) (PNIPAM), poly (vinylmethylether), poly (ethylene oxide), poly (ethyl (hydroxyethyl) cellulose), poly(2-ethyl oxazoline), polylactide (PLA), polyglycolide (PGA), poly(lactide-co-glycolide) PLGA, poly(e-caprolactone), polydiaoxanone, polyanhydride, trimethylene carbonate, poly(p-ydroxybutyrate), poly(g-ethyl glutamate), poly(DTH-iminocarbonate), poly(bisphenol A iminocarbonate), poly(orthoester) (POE), polycyanoacrylate (PCA), polyphosphazene, polyethyleneoxide (PEO), polyethylene glycol (PEG), polyacrylacid (PAA), polyacrylonitrile (PAN), polyvinylacrylate (PVA), polyvinylpyrrolidone (PVP), polyglycolic lactic acid (PGLA), poly(2-hydroxypropyl methacrylamide) (pHPMAm), poly(vinyl alcohol) (PVOH), PEG diacrylate (PEGDA), poly(hydroxyethyl methacrylate) (pHEMA), N-i sopropylacrylamide (NIPA), poly(vinyl alcohol) poly(acrylic acid) (PVOH-PAA), collagen, silk, fibrin, gelatin, hyaluron, cellulose, chitin, dextran, casein, albumin, ovalbumin, heparin sulfate, starch, agar, heparin, alginate, fibronectin, fibrin, keratin, pectin, elastin, ethylene vinyl acetate, polyethylene oxide, PEG or any of its derivatives, PLLA, PDMS, PIPA, PEVA, PILA, PEG styrene, Teflon RFE, FLPE, Teflon FEP, methyl palmitate, NIPA, polycarbonate, polyethersulfone, polycaprolactone, polymethyl methacrylate, polyisobutylene, nitrocellulose, medical grade silicone, cellulose acetate, cellulose acetate butyrate, polyacrylonitrile, PLCL, and/or chitosan.

In various embodiments, synthetic hydrogels are selected from the group consisting of hydrogels made from a hydrophilic monomer, hydrogels made from a hydrophilic polymer, hydrogels made from a hydrophilic copolymer, and combinations thereof.

Examples of hydrophilic monomer that may be used include, but are not limited to, 2-hydroxyethyl methacrylate, 2-hydroxyethyl acrylate, 2-hydroxyethyl methacrylamide, 2-hydroxyethyl acrylamide, N-2-hydroxyethyl vinyl carbamate, 2-hydroxyethyl vinyl carbonate, 2-hydroxypropyl methacrylate, hydroxyhexyl methacrylate, hydroxyoctyl methacrylate, acrylic acid, methacrylic acid, itaconic acid, fumaric acid, crotonic acid, maleic acid, monomethyl maleate ester, monoethyl maleate ester, monomethyl fumarate ester, monoethyl fumarate ester, (meth)acrylamide, crotonic amide, cinnamic amide, maleic diamide, fumaric diamide, methanethiole, ethanethiol, 1-propanethiol, butanethiol, tert-butyl mercaptan, pentanethiols, p-styrenesulfonic acid, vinylsulfonic acid, p-a-methylstyrenesulfonic acid, isoprene sulfonide and salts thereof.

Examples of hydrophilic polymer that may be used include, but are not limited to, polymers and oligomers of glycolide, lactide, polylactic acid, polyesters of a-hydroxy acids, including lactic acid and glycolic acid, such as the poly(a-hydroxy) acids including polyglycolic acid, poly-DL-lactic, poly-L-lactic acid, and terpolymers of DL-lactide and glycolide, e-caprolactone and e-caprolactone copolymerized with polyesters, polylactones and polycaprolactones including poly(e-caprolactone), poly(8-valerolactone) and poly (gamma-butyrolactone); polyanhydrides, polyorthoesters, other hydroxy acids, polydioxanone, collagen-hydroxyethylmethacrylate (HEMA), poly(hydroxylethyl methacrylate) (PHEMA), and other biologically degradable polymers that are non-toxic or are present as metabolites in the body. The above listed examples of hydrophilic polymers are also biodegradable.

The hydrogel composition can comprise an elastic modulus of at least, or of at most, 2 Pa, 3 Pa, 4 Pa, 5 Pa, 6 Pa, 7 Pa, 8 Pa, 9 Pa, 10 Pa, 20 Pa, 30 Pa, 40 Pa, 50 Pa, 60 Pa, 70 Pa, 80 Pa, 90 Pa, 100 Pa, 200 Pa, 300 Pa, 400 Pa, 500 Pa, 600 Pa, 700 Pa, 800 Pa, 900 Pa, 1000 Pa, 10000 Pa, 100000 Pa, or a number or a range between any two of these values. The polymer scaffold can be biodegradable, biocompatible and/or hydrophilic. The polymer scaffold can be a water-insoluble network of polymers.

The hydrogel composition can comprise a water content between about 10% and about 100% (e.g., 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values).

The polymer scaffold can comprise about 0.000000001%, to about 90% (e.g., 0.000000001%, 0.00000001%, 0.0000001%, 0.000001%, 0.00001%, 0.0001%, 0.001%, 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, or a number or a range between any two of these values) volume per volume (% v/v), weight per volume (% w/v) or weight per weight (% w/w) of the hydrogel composition.

The plurality of gas vesicles can comprise about 0.000000001% to about 90% (e.g., 0.000000001%, 0.00000001%, 0.0000001%, 0.000001%, 0.00001%, 0.0001%, 0.001%, 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, or a number or a range between any two of these values) volume per volume (% v/v), weight per volume (% w/v) or weight per weight (% w/w) of the hydrogel composition.

The plurality of payload molecules can comprise about 0.000000001% to about 90% (e.g., 0.000000001%, 0.00000001%, 0.0000001%, 0.000001%, 0.00001%, 0.0001%, 0.001%, 0.01%, 0.1%, 1%, 2%, 3%, 4, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, or a number or a range between any two of these values) volume per volume (% v/v), weight per volume (% w/v) or weight per weight (% w/w) of the hydrogel composition.

In some embodiments, less than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or a number or a range between any two of these values, of the plurality of payload molecules and/or the plurality of gas vesicles within the hydrogel composition are released from the hydrogel composition within a 24 hour period in the absence of exposure to ultrasound.

Porosity

The polymer scaffold can comprise a plurality of pores. The hydrogels can comprise porous structures, and the size and uniformity of the pores in the hydrogels can depend on, among other factors, the nature of the scaffold forming the structural basis of a given hydrogel, e.g., the composition of polymers forming the scaffold, the grade of cross-linking of polymers within the scaffold, and the concentration of the scaffold-forming polymers in the hydrogel, with higher densities typically associated with smaller pore size and vice versa. The size of the pores of a hydrogel can determine, in turn, how well a hydrogel can retain a given molecule, cell, particle, or controlled-release form. The term “pore size,” as used herein in the context of hydrogels, can refer to the diameter of the pores in a hydrogel scaffold. In some embodiments, the pore size is the average inner diameter of pores in a hydrogel. In other embodiments, the term can refer to the smallest or the largest inner diameter of a pore found in a given hydrogel. The pore size of some hydrogels are known to those of skill in the art, and the pore size of a hydrogel in question can be determined with no more than routine experimentation, e.g., by subjecting the hydrogel to an imaging assay of suitable resolution, e.g., a scanning microscopy assay, as described herein, or to a size exclusion assay with a series of molecules of known molecular weight and/or diameter. In some embodiments, the pore size of a hydrogel used in the context of this disclosure is about 10 nm to about 100 nm, about 50 nm to about 250 nm, about 250 nm to about 500 nm, or about 300 nm to about 700 nm. In some embodiments, the pore size of a hydrogel used in the context of this disclosure is about 10 nm, about 20 nm, about 30 nm, about 40 nm, about 50 nm, about 60 nm, about 70 nm, about 80 nm, about 90 nm, about 100 nm, about 200 nm, about 300 nm, about 400 nm, about 500 nm, about 600 nm, about 700 nm, about 800 nm, about 900 nm, or about 1000 nm. Pore sizes that are larger or smaller than the ones enumerated immediately above may be used in some embodiments.

The polymer scaffold can comprise a porosity of at least about 5% (e.g., 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or a number or a range between any two of these values). The polymer scaffold can comprise an average pore size in the range of about 1 nm to about 30000 nm (e.g., about 1 nm, 5 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm, 10 m, 20 μm, 30 μm, or a number or a range between any of these values).

The polymer scaffold can comprise an average pore size in the range of about 1 nm to about 100 nm (e.g., about 1 nm, 5 nm, 10 nm, 20 nm, 30 nm, 40 nm, 50 nm, 60 nm, 70 nm, 80 nm, 90 nm, 100 nm, or a number or a range between any of these values). The polymer scaffold can comprise a pore size in the range of about 1 nm to about 1,000 m (e.g., about 1 nm, 5 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 μm, 2 μm, 3 m, 4 μm, 5 μm, 6 μm, 7 μm, 8 μm, 9 μm, 10 μm, 20 μm, 30 μm, 70 μm, 100 μm, 300 m, 500 μm, 700 μm, 900 μm, 1000 μm, or a number or a range between any of these values) and a porosity in the range of about 5% to about 97% (e.g., 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or a number or a range between any two of these values).

In some embodiments, the average pore size of the hydrogels is smaller than the average size of a payload, e.g., drug or treatment. For example, if the hydrogel comprises a payload molecule with the average diameter being about 20-30 nm, then, in some embodiments, the average pore size of the hydrogel is less than 20-30 nm, including, for example, 100-200 μm. Payloads and hydrogel systems across clinically-relevant length scales are provided herein (e.g, Tables 1-2). Choosing an average pore size smaller than the average diameter of an payload to be encapsulated, here the controlled-release form of a payload molecule, ensures that the payload is effectively retained by the hydrogel scaffold and cannot easily diffuse or otherwise leak out of the hydrogel scaffold. In some embodiments, the cells to be encapsulated within the hydrogel scaffold are smaller in diameter than the average pore size of the hydrogel, which, in turn, is smaller than the average diameter of the controlled-release form of the respective payload molecule to be encapsulated. In some embodiments, cell adhesion, rather than hydrogel pore size, retains cells encapsulated in the gel within the hydrogel scaffold. In some embodiments, the polymer scaffold does not comprise pores more than about 10 nm, 20 nm, 30 nm, 40 nm, 50 nm, 60 nm, 70 nm, 80 nm, 90 nm, 100 nm, 200 nm, 300 nm, 400 nm, 500 nm, or a number or a range between any of these values, in size. The porosity of the polymer scaffold can be configured to retain embedded payload molecules of less than about 100,000 kDa, of less than about 10,000 kDa, of less than about 1,000 kDa, of less than about 100 kDa, of less than about 100 kDa, of less than about 10 kDa, or a number or a range between any two of these values. The porosity of the polymer scaffold can be configured to retain embedded payload molecules with a hydrodynamic radius of less than about 100 nm, about 80 nm, about 60 nm, about 40 nm, about 30 nm, about 20 nm, about 10 nm, about 5 nm, about 1 nm, or a number or a range between any two of these values. The average pore size of the polymer scaffold can be smaller than the average diameter of the payload molecules.

In Situ Hydrogels, Microgels, Degradable Hydrogels, and Cyrogels

Some embodiments of the hydrogel compositions provided herein relate to in situ hydrogels, microgels, cryogels, and/or degradable hydrogels.

In some embodiments, as for example in embodiments that relate to the in situ formation of a hydrogel in a dysfunctional tissue of a subject, preferred reactive moieties form a covalent bond under physiological conditions and do not produce any toxic side products when forming a covalent bond.

The term “physiological conditions,” as used herein, can refer to a range of chemical (e.g., pH, ionic strength), biochemical (e.g., enzyme concentrations), and physical (e.g., temperature, pressure) conditions that can be encountered in intracellular and extracellular fluids of tissues, such as, for example, in the intracellular and extracellular fluids of a subject. For most cells and tissues, the physiological pH ranges from about 7.0 to about 7.5, the physiological ionic strength ranges from about 50 mM to about 400 mM, the physiological temperature ranges from about 20° C. to about 42° C., and the physiological pressure ranges from about 925 mbar to about 1050 mbar.

Suitable chemistries for in situ hydrogel formation include, without limitation, boronate esterification (e.g., phenylboronate-salicylhydroxamate conjugation), click chemistry reactions (e.g., 1,3-dipolar cycloaddition), Diels-Alder reactions, amidation via modified Staudinger ligation, as well as chemistries yielding imine, oxime, and hydrazone linkages. In some embodiments the reactive moiety is an anhydride, such as, for example, and adipic anhydride, and the second reactive moiety is an aldehyde moiety, and these moieties react to form a hydrazone bond. In some such embodiments, the reactive moiety of a first polymer forms a covalent bond with a reactive moiety of a second polymer, thus linking the polymers. In some embodiments, a single polymer partaking in such a reaction is conjugated to or comprises a plurality of reactive moieties of the same type, thus allowing cross-linking of the reactant polymers. In the context of hydrogel formation, suitable reactive moieties are typically stable in water and in air, comprise nontoxic functional groups that react without toxic side products, and the bond-forming reaction kinetics are rapid or controllable.

In some embodiments, the reactive moiety is a click chemistry moiety. The term “click chemistry,” as used herein, can refer to a chemical philosophy introduced by K. Barry Sharpless of The Scripps Research Institute, describing chemistry tailored to generate covalent bonds quickly and reliably by joining small units comprising reactive groups together. Click chemistry does not refer to a specific reaction, but to a concept including reactions that mimic reactions found in nature. In some embodiments, click chemistry reactions are modular, wide in scope, give high chemical yields, generate non-toxic byproducts, are stereospecific, exhibit a large thermodynamic driving force >84 kJ/mol to favor a reaction with a single reaction product, and/or can be carried out under physiological conditions. In particular, click chemistry reactions that can be carried out under physiological conditions and that do not produce toxic or otherwise harmful side products are suitable for the generation of hydrogels in situ. Reactive moieties that can partake in a click chemistry reaction are well known to those of skill in the art, and include, but are not limited to alkyne and azide, alkene and tetrazole or tetrazine, or diene and dithioester. Other suitable reactive click chemistry moieties suitable for use in the context of polymer functionalization for hydrogel generation are known to those of skill in the art.

As used herein, the term “microparticle” can refer to a microscopic particle having a size measured in micrometers (m). Size of the microparticles may be characterized by their maximal dimension. The term “maximal dimension” as used herein can refer to the maximal length of a straight line segment passing through the center of a microparticle and terminating at the periphery. In the case of microspheres, the maximal dimension of a microsphere corresponds to its diameter. The term “mean maximal dimension” can refer to an average or mean maximal dimension of the microparticles, and may be calculated by dividing the sum of the maximal dimension of each microparticle by the total number of microparticles. Accordingly, value of maximal dimension may be calculated for microparticles of any shape, such as microparticles having a regular shape such as a sphere, a hemispherical, a cube, a prism, or a diamond, or an irregular shape. The particles provided herein can need not be spherical and can comprise, for example, a shape such as a cube, cylinder, tube, block, film, and/or sheet.

The maximal dimension of the hydrogel microparticle (e.g., microgel) formed may be in the range from about 1 μm to about 10000 μm, such as between about 50 m to about 150 μm, about 50 μm to about 100 μm, or about 50 μm to about 75 m. In various embodiments, the hydrogel microparticles formed are essentially monodisperse.

As used herein, the term “degradable hydrogel” can refer to a hydrogel having a structure which may decompose to smaller molecules under certain conditions, such as temperature, abrasion, pH, ionic strength, electrical voltage, current effects, radiation and biological means.

Thermal degradation can refer to the use of heat to apply to a material such that it decomposes into smaller molecules. Abrasion degradation or physical degradation can refer to the application of force or pressure on the material so as to break down the material into smaller components. Chemical degradation can refer to use of a chemical reagent which degrades a material such as hydrogel into smaller molecules through effects of pH or ionic strength of the solution, or through chemical reaction with the material. For example, a form of chemical degradation can be hydrolytic degradation, wherein gelatin undergoes hydrolytic degradation in the presence of water, and can form a product called collagen hydrolysate (CH), which can contain peptides with a mean molecular weight of 3-6 kDa.

Electrical degradation can refer to use of electrical current and/or voltage to pass through the material such that the material is decomposed. In radiation degradation, electromagnetic waves such as gamma and ultraviolet waves are used to degrade the material. The degradable hydrogel may also be degraded biologically, i.e. it is biodegradable. The term “biodegradable” can refer to a substance which can be broken down by microorganisms, or which spontaneously breaks down over a relatively short time (within 2-15 months) when exposed to environmental conditions commonly found in nature. For example, gelatin can be degraded by enzymes which are present in the body.

In some embodiments, degradation of the degradable hydrogel may take place over a time period ranging from a few seconds to a few days or months. The time period required for the hydrogel to degrade may depend on a few parameters, for example, constituent of hydrogel, such as type of hydrogel precursors or hydrogel-forming agents used and water content of the hydrogel, degree of cross-linking, temperature, pH, amount of aqueous medium present, and pressure during gelation. Under physiological conditions, that means in an animal body, degradation is in general about 2 months. This period may be extended by subjecting the hydrogel to a cross-linking agent as described above.

Cryogels are a class of materials with a highly porous interconnected structure that are produced using a cryotropic gelation (or cryogelation) technique. Cryogelation is a technique in which the polymerization-crosslinking reactions are conducted in quasi-frozen reaction solution. During freezing of the macromoner (MA-alginate) solution, the macromonomers and initiator system (APS/TEMED) expelled from the ice concentrate within the channels between the ice crystals, so that the reactions only take place in these unfrozen liquid channels. After polymerization and, after melting of ice, a porous material is produced whose microstructure is a negative replica of the ice formed. Ice crystals act as porogens. Pore size is tuned by altering the temperature of the cryogelation process. For example, the cryogelation process is typically carried out by quickly freezing the solution at −20° C. Lowering the temperature to, e.g., −80° C., would result in more ice crystals and lead to smaller pores.

Targeting Moieties

In some embodiments of the methods and compositions provided herein, the hydrogel composition can comprise one or more targeting moieties configured to bind a component of a target site of a subject. The one or more targeting moieties can be mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, and an RGD peptide or RGD peptide mimetic. The one or more targeting moieties can comprise one or more of the following: an antibody or antigen-binding fragment thereof, a peptide, a polypeptide, an enzyme, a peptidomimetic, a glycoprotein, a lectin, a nucleic acid, a monosaccharide, a disaccharide, a trisaccharide, an oligosaccharide, a polysaccharide, a glycosaminoglycan, a lipopolysaccharide, a lipid, a vitamin, a steroid, a hormone, a cofactor, a receptor, a receptor ligand, and analogs and derivatives thereof. The antibody or antigen-binding fragment thereof can comprise a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising complementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

The one or more targeting moieties can be configured to bind one or more of the following: CD3, CD4, CD5, CD6, CD7, CD8, CD9, CD10, CD11a, CD11b, CD11c, CD12w, CD14, CD15, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD46, CD47, CD48, CD49b, CD49c, CD51, CD52, CD53, CD54, CD55, CD56, CD58, CD59, CD61, CD62E, CD62L, CD62P, CD63, CD66, CD68, CD69, CD70, CD72, CD74, CD79, CD79a, CD79b, CD80, CD81, CD82, CD83, CD86, CD87, CD88, CD89, CD90, CD91, CD95, CD96, CD98, CD100, CD103, CD105, CD106, CD109, CD117, CD120, CD125, CD126, CD127, CD133, CD134, CD135, CD137, CD138, CD141, CD142, CD143, CD144, CD147, CD151, CD147, CD152, CD154, CD156, CD158, CD163, CD166, CD168, CD174, CD180, CD184, CDw186, CD194, CD195, CD200, CD200a, CD200b, CD209, CD221, CD227, CD235a, CD240, CD262, CD271, CD274, CD276 (B7-H3), CD303, CD304, CD309, CD326, 4-1BB, 5 AC, 5T4 (Trophoblast glycoprotein, TPBG, 5T4, Wnt-Activated Inhibitory Factor 1 or WAIF1), Adenocarcinoma antigen, AGS-5, AGS-22M6, Activin receptor like kinase 1, AFP, AKAP-4, ALK, Alpha integrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), B-lymphoma cell, bcr-abl, Bombesin, BORIS, C5, C242 antigen, CA125 (carbohydrate antigen 125, MUC16), CA-IX (CAIX, carbonic anhydrase 9), CALLA, CanAg, Canis lupus familiaris IL31, Carbonic anhydrase IX, Cardiac myosin, CCL11 (C-C motif chemokine 11), CCR4 (C-C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, CRIPTO, FCSF1R (Colony stimulating factor 1 receptor, CD 115), CSF2 (colony stimulating factor 2, Granulocyte-macrophage colony-stimulating factor (GM-CSF)), CTLA4 (cytotoxic T-lymphocyte-associated protein 4), CTAA16.88 tumor antigen, CXCR4 (CD 184), C—X—C chemokine receptor type 4, cyclic ADP ribose hydrolase, Cyclin B 1, CYP1B 1, Cytomegalovirus, Cytomegalovirus glycoprotein B, Dabigatran, DLL4 (delta-like—ligand 4), DPP4 (Dipeptidyl-peptidase 4), DR5 (Death receptor 5), E. coli Shiga toxin type-1, E. coli Shiga toxin type-2, ED-B, EGFL7 (EGF-like domain-containing protein 7), EGFR, EGFRII, EGFRvIII, Endoglin (CD 105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen l.F protein of respiratory syncytial virus, Frizzled receptor, Fucosyl GM1, GD2 ganglioside, G-28 (a cell surface antigen glycolipid), GD3 idiotype, GloboH, Glypican 3, N-glycolylneuraminic acid, GM3, GMCSF receptor a-chain, Growth differentiation factor 8, GP100, GPNMB (Transmembrane glycoprotein NMB), GUCY2C (Guanylate cyclase 2C, guanylyl cyclase C(GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-7, Influenza hemagglutinin, IgE, IgE Fc region, IGHE, IL-1, IL-2 receptor (interleukin 2 receptor), IL-4, IL-5, IL-6, IL-6R (interleukin 6 receptor), IL-9, IL-10, IL-12, IL-13, IL-17, IL-17A, IL-20, IL-22, IL-23, IL31RA, ILGF2 (Insulin-like growth factor 2), Integrins (α4, αᵤ β3, αvβ3, α₄β₇, α5β1, α6β4, α7β7, α11β3, α5β5, αvβ5), Interferon gamma-induced protein, ITGA2, ITGB2, KIR2D, LCK, Le, Legumain, Lewis-Y antigen, LFA-1 (Lymphocyte function-associated antigen 1, CD11a), LHRH, LINGO-1, Lipoteichoic acid, LIV1A, LMP2, LTA, MAD-CT-1, MAD-CT-2, MAGE-1, MAGE-2, MAGE-3, MAGE A1, MAGE A3, MAGE 4, MARTI, MCP-1, MIF (Macrophage migration inhibitory factor, or glycosylation inhibiting factor (GIF)), MS4A1 (membrane-spanning 4-domains subfamily A member 1), MSLN (mesothelin), MUC (Mucin 1, cell surface associated (MUC1) or polymorphic epithelial mucin (PEM)), MUC1-KLH, MUC16 (CA125), MCPI (monocyte chemotactic protein 1), MelanA/MARTI, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES 1, P21, p53 nonmutant, P97, Page4, PAP, Paratope of anti-(N-glycolylneuraminic acid), PAX3, PAX5, PCSK9, PDCD1 (PD-1, Programmed cell death protein 1, CD279), PDGF-Ra (Alpha-type platelet-derived growth factor receptor), PDGFR-J, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-α, TGF-β (Transforming growth factor beta), TGF-01, TGF-02 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TRP-2, Tyrosinase, VCAM-1 (CD 106), VEGF, VEGF-A, VEGF-2 (CD309), VEGFR-1, VEGFR2, or vimentin, WT1, XAGE 1, or cells expressing any insulin growth factor receptors, or any epidermal growth factor receptors.

### Gas Vesicles

Disclosed herein include hydrogel compositions comprising one or more types of gas vesicles (GVs). The plurality of gas vesicles can be capable of acting as steric diffusion blockers to the payload molecules embedded within the hydrogel. In some embodiments, the induction of gas vesicle collapse via US yields the formation of pores and/or percolation channels induced by US within the hydrogel composition. The dimensions of said pores and/or percolation channels can be larger than the dimensions of the payload molecules. The gas vesicles can be capable of impeding the diffusion of the payload molecules through the polymer scaffold in the absence of collapsing US.

Provided herein are gas-filled protein structures (GVPS), also referred to as “gas vesicles” (GVs), and related compositions methods and systems embedded within hydrogel compositions provided herein for remote-controlled, targeted payload delivery. The phrases “gas vesicles protein structure” or “GV”, “GVP”, “GVPS” or “Gas Vesicles” as used herein shall be given their ordinary meaning, and shall also refer to a gas-filled protein structure intracellularly expressed by certain bacteria or archea as a mechanism to regulate cellular buoyancy in aqueous environments. GVs are described in Walsby, A. E. ((1994). Gas vesicles. Microbiology and Molecular Biology Reviews, 58(1), 94-144) hereby incorporated by reference in its entirety. The term Gas Vesicle Structural (GVS) proteins as used herein indicates proteins forming part of a gas-filled protein structure intracellularly expressed by certain bacteria or archaea and can be used as a mechanism to regulate cellular buoyancy in aqueous environments. In particular, GVS shell comprises a GVS identified as gvpA or gvpB (herein also referred to as gvpA/B) and optionally also a GVS identified as gvpC. Exemplary microorganisms expressing or carrying gas vesicle protein structure include cyanobacteria such as Microcystis aeruginosa, Aphanizomenon flos aquae Oscillatoria agardhii, Anabaena, Microchaete diplosiphon and Nostoc; phototropic bacteria such as Amoebobacter, T. hiodiclyon, Pelodiclyon, and Ancalochloris; non phototropic bacteria such as Microcyclus aquaticus; Gram-positive bacteria such as Bacillus megalerium; Gram-negative bacteria such as Serratia; and archaea such as Haloferax mediterranei, Methanosarcina barkeri, Halobacteria salinarium as well as additional microorganisms identifiable by a skilled person. As described herein, GVs can be purified from their native genetic host organisms and/or be heterologously expressed in bacterial or eukaryotic cells (followed by purification).

In particular, a GV in the sense of the disclosure is a structure intracellularly expressed by bacteria or archaea forming a hollow structure wherein a gas is enclosed by a protein shell, which is a shell substantially made of protein (up at least 95% protein). In gas vesicles in the sense of the disclosure, the protein shell is formed by a plurality of proteins herein also indicated as Gyp proteins or Gvps, which are expressed by the bacteria or archaea and form in the bacteria or archaea cytoplasm a gas permeable and liquid impermeable protein shell configuration encircling gas. Accordingly, a protein shell of a GV is permeable to gas but not to surrounding liquid such as water. For example, GVs' protein shells exclude liquid water but permit gas to freely diffuse in and out from the surrounding media making them physically stable despite their usual nanometer size.

GV structures are typically nanostructures with widths and lengths of nanometer dimensions (in particular with widths of 45-250 nm and lengths of 100-800 nm) but can have lengths as large as the dimensions of a cell in which they are expressed, as will be understood by a skilled person. GVs and methods are described in Farhadi et al, Science, 2019, hereby incorporated by reference. In certain embodiments, the gas vesicles protein structure have average dimensions of 1000 nm or less, such as 900 nm or less, including 800 nm or less, or 700 nm or less, or 600 nm or less, or 500 nm or less, or 400 nm or less, or 300 nm or less, or 250 nm or less, or 200 nm or less, or 150 nm or less, or 100 nm or less, or 75 nm or less, or 50 nm or less. For example, the average diameter of the gas vesicles may range from 10 nm to 1000 nm, such as 25 nm to 500 nm, including 50 nm to 250 nm, or 100 nm to 250 nm. By “average” is meant the arithmetic mean.

GVs in the sense of the disclosure have different shapes depending on their genetic origins. For example, GVs in the sense of the disclosure can be substantially spherical, ellipsoid, cylindrical, or have other shapes such as football shape or cylindrical with cone shaped end portions depending on the type of bacteria providing the gas vesicles.

The term Gas Vesicle Structural (GVS) proteins as used herein indicates proteins forming part of a gas-filled protein structure intracellularly expressed by certain bacteria or archaea and can be used as a mechanism to regulate cellular buoyancy in aqueous environments. In particular, GVS shell comprises a GVS identified as gvpA or gvpB (herein also referred to as Gyp A/B) and optionally also a GVS identified as gvpC. GvpA is a structural protein that assembles through repeated unites to make up the bulk of GVs. GvpC is a scaffold protein with 5 repeat units that assemble on the outer shell of GVs. GvpC can be engineered to tune the mechanical and acoustic properties of GVs as well as act as a handle for appending moieties on to. A gvpC protein is a hydrophilic protein of a GV shell, which includes repetitions of one repeat region flanked by an N-terminal region and a C terminal region. The term “repeat region” or “repeat” as used herein with reference to a protein can refer to the minimum sequence that is present within the protein in multiple repetitions along the protein sequence without any gaps. Accordingly, in a gvpC multiple repetitions of a same repeat is flanked by an N-terminal region and a C-terminal region. In a same gvpC, repetitions of a same repeat in the gvpC protein can have different lengths and different sequence identity one with respect to another.

In some embodiments, the isolating GVs from a prokaryote of interest can be performed following methods to isolate gas vesicles as described in U.S. application Ser. No. 15/613,104, filed on Jun. 2, 2017. Isolating the protein for the protein shell of the GV and obtaining the related amino acidic sequence can be performed with tandem liquid chromatography mass-spectrometry alone or in combination with obtaining amino acid sequences of the isolated protein with wet lab techniques or from available databases comprising the sequences of the prokaryote of interest as well as additional techniques and approaches identifiable by a skilled person. Obtaining amino acid sequences of GV shell proteins of the prokaryote of interest can be performed by screening available databases of gene and protein sequences identifiable by a skilled person. Performing a sequence alignment of the sequences of the isolated GV proteins or proteins encoded in the genome of a prokaryote of interest can be performed (using Protein BLAST as described herein) against a Gyp A/B protein consensus sequence. In particular, a sequence alignment can be performed using Gyp A/B protein sequences from the closest phylogenetic relative to the prokaryote of interest.

The optional gvpC gene encodes for a gvpC protein which is a hydrophilic protein of a GV shell, including repetitions of one repeat region flanked by an N-terminal region and a C terminal region. The term “repeat region” or “repeat” as used herein with reference to a protein can refer to the minimum sequence that is present within the protein in multiple repetitions along the protein sequence without any gaps. Accordingly, in a gvpC multiple repetitions of a same repeat is flanked by an N-terminal region and a C-terminal region. In a same gvpC, repetitions of a same repeat in the gvpC protein can have different lengths and different sequence identity one with respect to another. In performing alignment steps sequence are identified as repeat when the sequence shows at least 3 or more of the characteristics described in U.S. application Ser. No. 15/663,635 published as US 2018/0030501 (incorporated herein by reference in its entirety) which also include additional features of gvpC proteins and the related identification.

The phrase “GV type” as used herein shall be given its ordinary meaning, and shall also refer to a gas vesicle having dimensions and shape resulting in distinctive mechanical, acoustic, surface and/or magnetic properties as will be understood by a skilled person upon reading of the present disclosure. In particular, a skilled person will understand that different shapes and dimensions will result in different properties in view of the indications in provided in U.S. application Ser. No. 15/613,104 and U.S. Ser. No. 15/663,600 and additional indications identifiable by a skilled person.

In some embodiments, GVs are capable of withstanding pressures of several kPa. but collapse irreversibly at a pressure at which the GV protein shell is deformed to the point where it flattens or breaks, allowing the gas inside the GV to dissolve irreversibly in surrounding media, herein also referred to as a critical collapse pressure, or selectable critical collapse pressure, as there are various points along a collapse pressure profile (e.g., peak acoustic pressure).

A collapse pressure profile (e.g., peak acoustic pressure) as used herein indicates a range of pressures over which collapse of a population of GVs of a certain type occurs. In particular, a collapse pressure profile in the sense of the disclosure comprise increasing acoustic collapse pressure values, starting from an initial collapse pressure value at which the GV signal/optical scattering by GVs starts to be erased to a complete collapse pressure value at which the GV signal/optical scattering by GVs is completely erased. The collapse pressure profile of a set type of GV is thus characterized by a mid-point pressure where 50% of the GVs of the set type have been collapsed (also known as the “midpoint collapse pressure”), an initial collapse pressure where 5% or lower of the GVs of the type have been collapsed, and a complete collapse pressure where at least 95% of the GVs of the type have been collapsed. In some embodiments herein described a selectable critical collapse pressure can be any of these collapse pressures within a collapse pressure profile, as well as any point between them. The critical collapse pressure profile of a GV is functional to the mechanical properties of the protein shell and the diameter of the shell structure. U.S. Patent Application Publication No. 2020/0164095 describes gas vesicles, protein variants and related compositions methods and systems for singleplexed and/or multiplexed ultrasonic methods (e.g., imaging of a target site in which a gas vesicle provides contrast for the imaging) which is modifiable by application of a selectable acoustic collapse pressure value of the gas vesicle, the content of which is hereby expressly incorporated by reference in its entirety.

In some embodiments herein described, it has been found that the critical collapse pressure is also functional to the manner in which the forces are applying the pressure to the GV shell. Accordingly, different ways of applying pressure on a set GVs result in different types of critical collapse pressures associated to the set GV. The term the “acoustic pressure” as used herein shall be given its ordinary meaning and shall also indicate the pressure exerted by a sound wave, such as ultrasound wave, propagating through a medium. In ultrasound imaging, this wave is typically generated by an ultrasound transducer, and the pressure resulting at any time and point in the medium is determined by transducer output and patterns of constructive and destructive interference, attenuation, reflection, refraction and diffraction. Ultrasound images are generated by transmitting one or more pulses into the medium and acquiring backscattered signals from the medium, which depend on medium composition, including the presence of contrast agents.

The acoustic collapse pressure profile (e.g., peak acoustic pressure) of a given GV type can be determined by imaging GVs with imaging ultrasound energy after collapsing portions of the given GV type population with a collapsing ultrasound energy (e.g. ultrasound pulses) with increasing peak positive pressure amplitudes to obtain acoustic pressure data point of acoustic pressure values, the data points forming an acoustic collapse curve. The acoustic collapse pressure function f(p) can be derived from the acoustic collapse curve by fitting the data with a sigmoid function such as a Boltzmann sigmoid function. An acoustic collapse pressure profile in the sense of the disclosure can include a set of initial collapse pressure values, a midpoint collapse pressure value and a set of complete collapse pressure values. The initial collapse pressures are the acoustic collapse pressures at which 5% or less of the GV signal is erased. A midpoint collapse pressure is the acoustic collapse pressure at which 50% of the GV signal is erased. Complete collapse pressures are the acoustic collapse pressures at which 95% or more of the GV signal is erased. The pressure can be peak pressure. In some embodiments, the peak pressure is peak positive pressure. In some embodiments, the peak pressure is peak negative pressure.

The plurality of gas vesicles can comprise an average cross-sectional diameter of about 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 μm, or a number or a range between any of these values. The plurality of gas vesicles can comprise a gas permeable protein vesicle wall.

The plurality of gas vesicles can be bacterially-derived gas vesicles and/or archaea-derived gas vesicles. The plurality of gas vesicles can be derived from a variety of species, such as, for example, species of Anabaena bacteria, Halobacterium salinarum, and/or Bacillus megaterium.

The plurality of gas vesicles can be capable of gas vesicle collapse. Gas vesicle collapse can comprise an at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values) reduction in volume of one or more gas vesicles of the plurality of gas vesicles. Gas vesicle collapse can comprise an at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values) reduction in acoustic contrast of one or more gas vesicles of the plurality of gas vesicles. In some embodiments, the upper limit of volume reduction per GV is 99.9% or about 1000-fold.

In some embodiments, the gas vesicle collapse increases the diffusivity of the hydrogel material by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values). In some embodiments, the gas vesicle collapse increases the porosity of the hydrogel composition by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values). In some embodiments, the gas vesicle collapse increases the rate of payload molecule release from the hydrogel composition by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values). The percentage of noncollapsed gas vesicles within the hydrogel composition and diffusivity of the hydrogel composition can be inversely related.

In some embodiments, the gas vesicles reduce the diffusivity of the hydrogel composition by at least 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values) as compared a hydrogel composition which does not comprise the gas vesicles.

The plurality of gas vesicles can comprise clustered gas vesicles. In some embodiments, the clustering of gas vesicles is achieved without the use of a cross-linker. For example, in some embodiments, clustering of gas vesicles can be accomplished via van der waals interactions, electrostatic interactions and/or depletion forces. The plurality of gas vesicles can comprise a clustering moiety configured to form clustered gas vesicles. The clustered gas vesicles can comprise aggregates of two or more gas vesicles bound to each other via one or more clustering moieties. The clustering moiety can comprise biotin, avidin, streptavidin, heparin, an aptamer, a click-chemistry moiety, digoxigenin, anti-digoxigenin, dinitrophenol, anti-dinitrophenol. primary amine(s), carboxyl(s), hydroxyl(s), aldehyde(s), ketone(s), or any combination thereof. The clustered gas vesicles can comprise at least one cross-sectional dimension of between about 100 nm and about 100 μm (e.g., 100 nm, 200 nm, 300 nm, 400 nm, 500 nm, 600 nm, 700 nm, 800 nm, 900 nm, 1 μm, 2 μm, 3 μm, 4 μm, 5 m, 6 μm, 7 μm, 8 μm, 9 μm, 10 μm, 20 μm, 30 μm, 70 μm, 100 am, or a number or a range between any of these values).

The clustered gas vesicles can comprise a largest dimension between about 100 nm about 10 μm (e.g., about 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm, 10 m, or a number or a range between any of these values). The clustered gas vesicles can comprise a fractal dimension between about 1 and about 3 (e.g., about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.8, 3.0, or a number or a range between any of these values). The collapse of clustered gas vesicles can lead to the formation of pores and/or percolation channels within the hydrogel composition that can be at least 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values) larger in at least one dimension as compared to the formation of pores and/or percolation channels within a hydrogel composition wherein the gas vesicles are not clustered.

The plurality of gas vesicles can comprise a first collapse pressure profile. The first collapse pressure profile can comprise a collapse function from which a gas vesicle collapse amount can be determined for a given pressure value (e.g., peak acoustic pressure). The first collapse pressure profile can comprise a first initial collapse pressure where about 5% or lower of the plurality of gas vesicles are collapsed, a first midpoint collapse pressure where about 50% of the plurality of gas vesicles are collapsed, a first complete collapse pressure where at least about 95% of the plurality of gas vesicles are collapsed, any pressure between the first initial collapse pressure and the first midpoint collapse pressure, and any pressure between the first midpoint collapse pressure and the first complete collapse pressure. A first selectable collapse pressure can be any collapse pressure within the first collapse pressure profile. A first selectable collapse pressure can be selected from the first collapse pressure profile at a value between about 0.05% collapse of the plurality of gas vesicles and about 95% collapse of the plurality of gas vesicles. A first selectable collapse pressure can be equal to or greater than the first initial collapse pressure. A first selectable collapse pressure can be equal to or greater than the first midpoint collapse pressure. A first selectable collapse pressure can be equal to or greater than the first complete collapse pressure.

The hydrogel composition can comprise: a plurality of secondary gas vesicles. The secondary gas vesicles can comprise different mechanical, acoustic, and/or surface properties as compared to the gas vesicles. The plurality of secondary gas vesicles can comprise a second collapse pressure profile. The second collapse pressure profile can comprise a collapse function from which a secondary gas vesicle collapse amount can be determined for a given pressure value (e.g., peak acoustic pressure). The first collapse pressure profile and the second collapse pressure profile can be different. In some embodiments, the first collapse pressure profile and/or second collapse pressure profile has been configured by engineering a gas vesicle protein C (GvpC) protein of the gas vesicles and/or the secondary gas vesicles, or has been configured by any of the other methods provided herein. In some embodiments, a midpoint of the second collapse profile has a higher pressure component than a midpoint of the first collapse profile. The second collapse pressure profile can comprise a second initial collapse pressure where about 5% or lower of the plurality of secondary gas vesicles are collapsed, a second midpoint collapse pressure where about 50% of the plurality of secondary gas vesicles are collapsed, a second complete collapse pressure where at least about 95% of the plurality of secondary gas vesicles are collapsed, any pressure between the second initial collapse pressure and the second midpoint collapse pressure, and any pressure between the second midpoint collapse pressure and the second complete collapse pressure. A second selectable collapse pressure can be any collapse pressure within the second collapse pressure profile. A second selectable collapse pressure can be selected from the second collapse pressure profile at a value between about 0.05% collapse of the plurality of secondary gas vesicles and about 95% collapse of the plurality of secondary gas vesicles. A second selectable collapse pressure can be equal to or greater than the second initial collapse pressure. A second selectable collapse pressure can be equal to or greater than the second midpoint collapse pressure. A second selectable collapse pressure can be equal to or greater than the second complete collapse pressure. The gas vesicles can comprise one or more moieties configured to bind the polymer scaffold. The fourth US pulse(s) can comprise a pressure value less than the second selectable collapse pressure value. The sixth US pulse(s) can comprise a pressure value less than the second selectable collapse pressure value.

Engineered GVs and methods of tuning the acoustic properties thereof are provided in U.S. Patent Application Publication No. 2020/0164095, the content of which is incorporated herein by reference in its entirety. In some embodiments, the GVs can be engineered to modulate the GV mechanical, acoustic, surface and targeting properties in order to achieve enhanced harmonic responses and multiplexed imaging to be better distinguished from background tissues. In some embodiments herein described Gas vesicles protein structures can be provided by Gyp genes endogenously expressed in bacteria or archaea. Endogenous expression can refer to expression of Gyp proteins forming the protein shell of the GV in bacteria or archaea that naturally produce gas vesicles encoded (e.g. in their genome or native plasmid DNA). Gyp proteins expressed by bacteria or archaea typically include two primary structural proteins, here also indicated as GvpA and GvpC, and several putative minor components and chaperones as would be understood by a person skilled in the art. In some embodiments, heterologously expressed Gyp proteins to provide a GV type have independently at least 50% sequence identity, preferably at least 80%, more preferably at least 90%, most preferably at least 95% sequence identity compared to a reference sequence of corresponding Gyp protein using one of the alignment programs described using standard parameters.

In bacteria or archaea expressing GVs, the Gyp proteins forming a GV's protein shell are encoded by a cluster of 8 to 14 different genes depending on the host bacteria or archaea, as will be understood by a skilled person.

U.S. Patent Application Publication No. 2018/0030501 describes hybrid gas vesicle gene cluster (GVGC) configured for expression in a prokaryotic host comprising gas vesicle assembly (GVA) genes native to a GVA prokaryotic species and capable of being expressed in a functional form in the prokaryotic host, as well as one or more gas vesicle structural (GVS) genes native to one or more GVS prokaryotic species, at least one of the one or more GVS prokaryotic species different from the GVA prokaryotic species, and related gas vesicle reporting (GVR) genetic circuits, genetic, vectors, engineered cells, and related compositions methods and systems to produce GVs, hybrid GVGC and/or image a target site, the content of which is hereby expressly incorporated by reference in its entirety. The term “Gas Vesicle Genes Cluster” or “GVGC” as described herein indicates a gene cluster encoding a set of GV proteins capable of providing a GV upon expression within a bacterial or archaeal cell. The term “gene cluster” as used herein means a group of two or more genes found within an organism's DNA that encode two or more polypeptides or proteins, which collectively share a generalized function or are genetically regulated together to produce a cellular structure and are often located within a few thousand base pairs of each other. The size of gene clusters can vary significantly, from a few genes to several hundred genes. Portions of the DNA sequence of each gene within a gene cluster are sometimes found to be similar or identical; however, the resulting protein of each gene is distinctive from the resulting protein of another gene within the cluster. Genes found in a gene cluster can be observed near one another on the same chromosome or native plasmid DNA, or on different, but homologous chromosomes. An example of a gene cluster is the Hox gene, which is made up of eight genes and is part of the Homeobox gene family. In the sense of the disclosure, gene clusters as described herein also comprise gas vesicle gene clusters, wherein the expressed proteins thereof together are able to form gas vesicles.

In some embodiments herein described, identification of a gene cluster encoding for Gyp proteins in a bacteria or archaea can be performed for example by isolating the GVs from the bacteria or archaea, isolating the protein for the protein shell of the GV and derive the related amino acidic sequence with methods and techniques identifiable by a skilled person. The sequence of the genes encoding for the Gyp proteins can then be identified by method and techniques known in the art. For example, gas vesicle gene clusters can also be identified by persons skilled in the art by performing gene sequencing or partial- or whole-genome sequencing of organisms using wet lab and in silico molecular biology techniques known to those skilled in the art. As understood by those skilled in the art, gas vesicle gene clusters can be located on the chromosomal DNA or native plasmid DNA of microorganisms. After performing DNA or cDNA isolation from a microorganism, the polynucleotide sequences or fragments thereof or PCR-amplified fragments thereof can be sequenced using DNA sequencing methods such as Sanger sequencing, DNASeq, RNASeq, whole genome sequencing, and other methods known in the art using commercially available DNA sequencing reagents and equipment, and then the DNA sequences analyzed using computer programs for DNA sequence analysis known to skilled persons.

Gas vesicle gene cluster genes can also be identified in DNA sequence databases such as GenBank, EMBL, DNA Data Bank of Japan, and others. Gas vesicle gene cluster gene sequences in databases such as those above can be searched using tools such as NCBI Nucleotide BLAST and the like, for gas vesicle gene sequences and homologs thereof, using gene sequence query methods known to those skilled in the art.

Representative examples of endogenously expressed GVs are the gas vesicle protein structure produced by the Cyanobacterium Anabaena flos-aquae (Ana GVs), and the Halobacterium Halobacterium salinarum (Halo GVs). In particular, Ana GVs are cone-tipped cylindrical structures with a diameter of approximately 140 nm and length of up to 2 μm and in particular 200-800 nm or longer, encoded by a cluster of nine different genes, including the two primary structural proteins, GvpA and GvpC, and several putative minor components and putative chaperones as would be understood by a person skilled in the art. Halo GVs are typically spindle-like structures with a maximal diameter of approximately 250 nm and length of 250-600 nm, encoded by a cluster of fourteen different genes, including the two primary structural proteins, GvpA and GvpC, and several putative minor components and putative chaperones as would be understood by a person skilled in the art

In some embodiments herein described Gas vesicles protein structures can be provided by Gyp genes heterologously expressed in bacteria or archaea or eukaryotes (e.g., animal cells). Said animal cells include, for example, human and Chinese Hamster cells, using the compositions and methods disclosed in Farhadi et al, Science, 2019, hereby incorporated by reference. Heterologous expression can refer to expression of Gyp proteins in any species that either does not naturally produce gas vesicles, or where its natural production of gas vesicles has been suppressed, for example through genetic knock-out of the genes encoding Gyp proteins, and where foreign DNA encoding gas vesicle genes is introduced into the organism to persist as a plasmid or integrate into the genome.

In some embodiments, heterologously expressed Gyp genes can comprise genes encoding for corresponding Gyp proteins which are naturally occurring or have sequences having at least 50% identity with naturally occurring Gyp proteins.

As used herein, “sequence identity” or “identity” in the context of two nucleic acid or polypeptide sequences makes reference to the nucleotide bases or residues in the two sequences that are the same when aligned for maximum correspondence over a specified comparison window. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule.

A functionally equivalent residue of an amino acid used herein typically can refer to other amino acid residues having physiochemical and stereochemical characteristics substantially similar to the original amino acid. The physiochemical properties include water solubility (hydrophobicity or hydrophilicity), dielectric and electrochemical properties, physiological pH, partial charge of side chains (positive, negative or neutral) and other properties identifiable to a person skilled in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

Heterologous expression of GVs in bacteria or archaea that do not express GVs can be performed by cloning one or more polynucleotides encoding naturally occurring Gyp proteins or homologs thereof that are required for production of GVs (comprising gvpA, gvpC, and other proteins known to those skilled in the art and described herein) into one or more suitable expression plasmids or vectors, and expressing the heterologous GV proteins in the bacteria or archaea. Polynucleotides encoding GV protein genes can be cloned using commercially available reagents from vendors such as Qiagen, Invitrogen, Applied Biosystems, Promega, and others, following standard molecular biology methods known in the art, such as those described herein. As would be understood by those skilled in the art, polynucleotides encoding GV protein genes can be obtained from several different sources. For example, polynucleotides encoding GV proteins can be obtained by isolating genomic DNA or cDNA encoding GV proteins from microorganisms whose genomes encode GV proteins genes, and/or express GV proteins RNA. RNA can be isolated from a cell that expresses GV proteins genes, and cDNA produced by reverse transcription using standard techniques and commercial kits. Genomic DNA can be purified from the cell, and cDNA or genomic DNA encoding one or more GV proteins isolated, following methods known to those in the art. Alternatively, polynucleotides comprising one or more gas vesicle genes can be synthesized using oligonucleotide and polynucleotide synthetic methods known in the art. PCR-based amplification of one or more GV protein genes can be performed using appropriately designed primer pairs (e.g. using PrimerDesign or other programs known to those skilled in the art). PCR-based amplification can be followed by ligation (e.g. using T4 DNA ligase) of a polynucleotide encoding gas vesicle gene amplicon into an appropriate expression cassette in a plasmid suitable for propagation in bacteria or other cells, such as transformation-competent E. co/iDH5alpha, followed by growth of transformed cell cultures, purification of the plasmid for confirmation of the cloned enzyme by DNA sequence analysis, among other methods known to those skilled in the art. Expression vectors can comprise plasmid DNA, viral vectors, or non-viral vectors, among others known to those skilled in the art, comprising appropriate regulatory elements such as promoters, enhancers, and post-transcriptional and post-translational regulatory sequences that are compatible with the bacteria or archaea heterologously expressing the GV, as would be understood by a skilled person. Promoters can be constitutively active or inducible. Exemplary inducible expression systems comprise IPTG-inducible expression as described in the Examples.

In some embodiments, where one or more Gyp proteins are expressed heterologously to form GVs in microorganisms other that the native host, the related sequence can be optimized for expression in the heterologous host microorganism as will be understood by a skilled person.

In particular, in some embodiments described herein, wherein GV is produced heterologously production of a GV gvpc gene sequences can be codon-optimized for expression in one or more microorganism of choice such as Escherichia coli, according to methods identifiable by a skilled person. As would be understood by those skilled in the art, the term “codon optimization” as used herein can refer to the introduction of synonymous mutations into codons of a protein-coding gene in order to improve protein expression in expression systems of a particular organism, such as E. coli in accordance with the codon usage bias of that organism. The term “codon usage bias” can refer to differences in the frequency of occurrence of synonymous codons in coding DNA. The genetic codes of different organisms are often biased towards using one of the several codons that encode a same amino acid over others-thus using the one codon with, a greater frequency than expected by chance. Optimized codons in microorganisms, such as Escherichia coli or Saccharomyces cerevisiae, reflect the composition of their respective genomic tRNA pool. The use of optimized codons can help to achieve faster translation rates and high accuracy.

In the field of bioinformatics and computational biology, many statistical methods have been proposed and used to analyze codon usage bias. Methods such as the ‘frequency of optimal codons’ (Fop), the Relative Codon Adaptation (RCA) or the ‘Codon Adaptation Index’ (CAI) are used to predict gene expression levels, while methods such as the ‘effective number of codons’ (Nc) and Shannon entropy from information theory are used to measure codon usage evenness. Multivariate statistical methods, such as correspondence analysis and principal component analysis, are widely used to analyze variations in codon usage among genes. There are many computer programs to implement the statistical analyses enumerated above, including CodonW, GCUA, INCA, and others identifiable by those skilled in the art. Several software packages are available online for codon optimization of gene sequences, including those offered by companies such as GenScript, EnCor Biotechnology, Integrated DNA Technologies, ThermoFisher Scientific, among others known those skilled in the art. Those packages can be used in providing Gyp proteins with codon ensuring optimized expression in various cell systems as will be understood by a skilled person.

A representative example of heterologous GVs is the E. coli expressing a heterologous GV gene cluster from Bacillus megaterium (Mega). Mega GVs are typically cone-tipped cylindrical structures with a diameter of approximately 73 nm and length of 100-600 nm, encoded by a cluster of eleven or fourteen different genes, including the primary structural protein, GvpA, and several putative minor components and putative chaperones as would be understood by a person skilled in the art.

In some embodiments, the GVs can be engineered to modulate their mechanical, acoustic, surface and targeting properties in order to achieve enhanced harmonic responses and multiplexed imaging to be better distinguished from background tissues. In particular in those embodiments, a GV can be engineered to provide a variant GvpC protein and corresponding variant GV type and/or to provide a variant GV type with a modified amount of native or engineered GvpC protein on the protein shell of the GV.

A GvpC protein is a hydrophilic protein encoded by a gene of the GV gene cluster, which includes repetitions of one repeat region flanked by an N-terminal region and a C terminal region. The term “repeat region” or “repeat” as used herein with reference to a protein can refer to the minimum sequence that is present within the protein in multiple repetitions along the protein sequence without any gaps. Accordingly, in a GvpC multiple repetitions of a same repeat is flanked by an N-terminal region and a C-terminal region. In a same GvpC, repetitions of a same repeat in the GvpC protein can have different lengths and different sequence identity one with respect to another.

In some embodiments, the engineered GvpC variants are obtained by further linking the native GvpC protein to one or more other proteins, polypeptides, or domains to form a recombinant fusion protein. In some embodiments of methods and systems and related compositions herein described one or more GVs (including variants GVs) can be engineered to include tags peptides and/or functional group to provide the GV with additional functionalities. In particular in some embodiments GVs can be functionalized through genetic and/or chemical modification of a Gyp protein (including variants GvpC protein described herein).

In particular, some embodiments here described tags and/or functional groups can be added through chemical or genetic modification of a GvpC protein or a variant thereof in accordance with the present disclosure of a set type of GV and/or through chemical modification of another Gyp protein of the set type GV.

In some embodiments, functionalization of a Gyp protein can be performed by reacting one or more GVs with one or more compounds to allow attachment and presentation of a functional group on the protein shell of the one or more GVs.

The term “functional group” as used herein indicates specific groups of atoms within a molecular structure that are responsible for the characteristic chemical reactions of that structure. Exemplary functional groups include hydrocarbons, groups containing halogen, groups containing oxygen, groups containing nitrogen and groups containing phosphorus and sulfur all identifiable by a skilled person. In particular, functional groups in the sense of the present disclosure include a carboxylic acid, amine, triarylphosphine, azide, acetylene, sulfonyl azide, thio acid and aldehyde. In particular, for example, the first functional group and the second functional group can be selected to comprise the following binding partners: carboxylic acid group and amine group, azide and acetylene groups, azide and triarylphosphine group, sulfonyl azide and thio acid, and aldehyde and primary amine. Additional functional groups can be identified by a skilled person upon reading of the present disclosure. As used herein, the term “corresponding functional group” can refer to a functional group that can react to another functional group. Thus, functional groups that can react with each other can be referred to as corresponding functional groups.

The term “present” as used herein with reference to a compound or functional group indicates attachment performed to maintain the chemical reactivity of the compound or functional group as attached. Accordingly, a functional group presented on a GV shell and/or a Gyp protein thereof, is able to perform under the appropriate conditions the one or more chemical reactions that chemically characterize the functional group.

In some embodiments, the functionalization of the GvpC can be performed by chemical conjugation to a GvpC protein shell of moieties such as lysine residues and/or amine-reactive crosslinkers such as sulfo-N-hydroxysuccinimide esters (Sulfo-NHS). Depending on the application, the desired extent of labeling can be tuned by varying the molar ratio of Sulfo-NHS to GVs and by changing the incubation time as will be understood by a skilled person. Additional, chemical moieties including polymers (e.g. polyethylene glycol), fluorophores and small molecules (e.g. biotin) which can be conjugated methods identifiable. Biotinylated GVs can subsequently react with streptavidin or avidinated antibodies. Either dialysis or buoyancy purification can be used to separate the labeled GVs from excess reactants.

In some embodiments methods to functionalize GVs can be performed by genetically engineering a GvpC protein of the GV shell to include one or more protein tags. The term “tag” as used herein means protein tags comprising peptide sequences introduced onto a recombinant protein. Tags can be removable by chemical agents or by enzymatic means, such as proteolysis or splicing. Tags can be attached to proteins for various purposes: Affinity tags are appended to proteins so that they can be purified from their crude biological source using an affinity technique. These include chitin binding protein (CBP), and the poly(His) tag. The poly(His) tag is a widely-used protein tag; it binds to metal matrices. Chromatography tags can be used to alter chromatographic properties of the protein to afford different resolution across a particular separation technique. Often, these consist of polyanionic amino acids, such as FLAG-tag. Epitope tags are short peptide sequences which are chosen because high-affinity antibodies can be reliably produced in many different species. These are usually derived from viral genes, which explain their high immunoreactivity. Epitope tags include V5-tag, Myc-tag, HA-tag and NE-tag. These tags are particularly useful for western blotting, immunofluorescence and immunoprecipitation experiments, although they also find use in antibody purification. Protein tags can allow specific enzymatic modification (such as biotinylation by biotin ligase) or chemical modification (such as reaction with FlAsH-EDT2 for fluorescence imaging). Tags can be combined, in order to connect proteins to multiple other components. However, with the addition of each tag comes the risk that the native function of the protein may be abolished or compromised by interactions with the tag. Therefore, after purification, tags are sometimes removed by specific proteolysis (e.g. by TEV protease, Thrombin, Factor Xa or Enteropeptidase).

For example in some embodiments described herein, a GvpC protein of a GV can be engineered to attach one or more protein tags or polypeptide tags while optionally substantially altering the acoustic collapse pressure of a GV shell comprising the engineered GvpC as compared to a GV shell of a same non-engineered GvpC. The term “substantially alter” or “substantially decrease” as used herein means a decrease of more than 10% in acoustic collapse pressure, preferably more than 20% in acoustic collapse pressure.

Addition of a functional moiety comprised in a tag to a gvpC or a variant gvpC can be obtained through different approaches identifiable by a skilled person. For example, in some embodiments, an addition of a tag to a gvpC or variant gvpC can be performed at a protein level by first providing the gvpC protein or variant gvpC protein and the protein tag and then performing the insertion into a N-terminal or a C-terminal region by breaking a peptide bond between two adjacent amino acids of the gvpC or variant gvpC N-terminal region of C-terminal region and then forming new peptide bonds between the gvpC or variant gvpC and the protein tag, as described above. For example, the gvpC or variant gvpC can be digested with a protease to break a peptide bond between two adjacent amino acids in the gvpC or variant gvpC N-terminal region or C-terminal region, followed by insertion of the protein tag between the previously adjacent amino acids of the N-terminal region or C-terminal region, for example using native chemical ligation methods known to those skilled in the art. In other embodiments, a protein tag can be fused to a C-terminus or an N-terminus of a gvpC protein or a variant gvpC protein using native chemical ligation methods known in the art.

In some embodiments, the gas vesicles are Engineered Harmonic GV Variants (hGVs). Methods and compositions employing Gas Vesicles (GVs) and Engineered Harmonic GV Variants (hGVs) have been described in U.S. Patent Application Publication No. 2019/0314001, the content of which is hereby expressly incorporated by reference in its entirety. Generally, natural GVs behave as linear ultrasound scatterers at acoustic pressures above and below 300 kPa. Harmonic GV variants (hGVs) can be engineered, however, with fewer stiffening proteins so as to buckle and scatter higher harmonics at peak acoustic pressures of 300 kPa or higher. The bucking threshold of 300 kPa corresponds to a mechanical index of 0.08, which is below the FDA safety requirement of 1.9. Some examples of molecular engineered hGVs that buckle and scatter higher harmonics at acoustic pressures of 320 kPa are discussed in Lakshmanan A., Farhadi, A., Nety, Lee-Gosselin, S. P., A., Bourdeau, R. W., Maresca, D., and Shapiro, M. G., “Molecular Engineering of Acoustic Protein Nanostructures,” ACS Nano 10, 7314 (2016) and in Maresca, D., Lakshmanan, A., Lee-Gosselin, A., Melis, J. M., Ni, Y. L., Bourdeau, R. W., Kochmann, D. M., and Shapiro, M. G., “Nonlinear Ultrasound Imaging of Nanoscale Acoustic Biomolecules,” Appl. Phys. Lett. 110, 073704 (2017), both of which are hereby incorporated by reference in their entireties. In certain aspects, hGVs can be engineered to remove and/or alter one or more of the stiffening proteins to engineer hGVs that buckle, collapse, and/or cavitate at a particular acoustic pressure threshold.

Engineered harmonic gas vesicles (hGVs) can be engineered and harvested from host bacteria or archaea using various techniques. In one example, Anabaena GVs are cultured and transferred to sterile separating funnels, and the buoyant cells allowed to float to the top and separated from the spent media over a 48 hour period. The GVs are then harvested using hypertonic lysis. Purification can be performed by repeated centrifugally-assisted flotation followed by resuspension. Wild-type Ana GVs are then stripped of their outer GvpC layer by treatment with x-M urea solution to obtain hGVs. Next, two rounds of centrifugally-assisted flotation are followed by removal of the subnatant layer to ensure complete removal of native GvpC.

The hGVs vary in shape depending on the type of genetic host. In some cases, the hGVs are ellipsoid in shape or cylindrical with cone-shaped end portions. The diameter and length of the hGVs also varies. Typically hGVs have a diameter that ranges from about 115 nm to about 302 nm. In some cases, hGVs have a diameter of 60 nm. In some cases, hGVs have a length that ranges from about 440 nm to about 600 nm. In other cases, hGVs have a length that ranges from about 287 nm to about 513 nm. In yet other cases, hGVs have a length that ranges from about 150 nm to about 348 nm.

In certain aspects, hGVs have been engineered with a buckling threshold in a range between 200 kPa to 1000 kPa. In one aspect, hGVs have been engineered with a buckling threshold of about 400 kPa. In another aspect, hGVs have been engineered with a buckling threshold of about 600 kPa. In certain aspects, hGVs have been engineered with a collapse and/or cavitation threshold in a range between 150 kPa to 2000 kPa. In one aspect, hGVs have been engineered with a collapse and/or cavitation threshold of about 600 kPa. In another aspect, hGVs have been engineered with a collapse and/or cavitation threshold of about 900 kPa.

### Payload

The hydrogel compositions disclosed herein can comprise a plurality of payloads (e.g., payload molecules). More than one type of payload can be situated within (e.g., embedded within) the polymer matrix of the hydrogel composition. For example, a hydrogel composition can comprise a first payload and a second payload (e.g., a secondary payload) which can be different in size, shape, and/or intended purpose. The plurality of payload molecules can comprise a molecular weight of less than about 100,000 kDa, of less than about 10,000 kDa, of less than about 1,000 kDa, of less than about 100 kDa, of less than about 100 kDa, or of less than about 10 kDa, or a number or a range between any of these values.

The plurality of payload molecules can comprise a hydrodynamic radius of about 5 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 μm, or a number or a range between any of these values. The payload molecules provided herein encompass pharmaceutically-acceptable salts thereof.

The plurality of payload molecules can comprise a therapeutic agent useful for treating a disease of the GI tract. The disease of the GI tract can be an inflammatory bowel disease. The therapeutic agent useful for treating inflammatory bowel disease can comprise one of the following classes of compounds: 5-aminosalicyclic acids, corticosteroids, thiopurines, tumor necrosis factor-alpha blockers and JAK inhibitors. The payload molecules can comprise an immunosuppressant. The therapeutic agent useful for treating inflammatory bowel disease can comprise one or more of Prednisone, Humira, Lialda, Imuran, Sulfasalazine, Pentasa, Mercaptopurine, Azathioprine, Apriso, Simponi, Enbrel, Humira Crohn's Disease Starter Pack, Colazal, Budesonide, Azulfidine, Purinethol, Proctoso! HC, Sulfazine EC, Delzicol, Balsalazide, Hydrocortisone acetate, Infliximab, Mesalamine, Proctozone-HC, Sulfazine, Orapred ODT, Mesalamine, Azasan, Asacol HD, Dipentum, Prednisone Intensol, Anusol-HC, Rowasa, Azulfidine EN-tabs, Veripred 20, Uceris, Adalimumab, Hydrocortisone, Colocort, Pediapred, Millipred, Azathioprine injection, Prednisolone sodium phosphate, Flo-Pred, Aminosalicylic acid, ProctoCream-HC, 5-aminosalicylic acid, Millipred DP, Golimumab, Prednisolone acetate, Rayos, Proctocort, Paser, Olsalazine, Procto-Pak, Purixan, Cortenema, Giazo, Vedolizumab, Entyvio, Micheliolide, and Parthenolide. The plurality of payload molecules can comprise a single dose or a plurality of doses.

The plurality of payload molecules can comprise a small molecule, a nucleic acid, a cell, a vector, a saccharine, a monosaccharide, a disaccharide, a trisaccharide, an oligosaccharide, a polysaccharide, a peptide, a protein, a peptide analog, a peptidomimetic, an antibody or antigen-binding fragment thereof, an antisense oligonucleotide, an siRNA, an shRNA, a ribozyme, an aptamer, a microRNA, a pre-microRNA, plasmid DNA, modified RNA, or any combination thereof. In some embodiments, the plurality of payload molecules can treat and/or prevent a disease or disorder.

A payload molecule can comprise a compound selected from the group consisting of small organic or inorganic molecules; saccharines; monosaccharides; disaccharides; trisaccharides; oligosaccharides; polysaccharides; peptides; proteins, peptide analogs and derivatives; peptidomimetics; antibodies (polyclonal and monoclonal); antigen binding fragments of antibodies; nucleic acids, e.g., oligonucleotides, antisense oligonucleotides, siRNAs, shRNAs, ribozymes, aptamers, microRNAs, pre-microRNAs, plasmid DNA (e.g. condensed plasmid DNA), modified RNA, nucleic acid analogs and derivatives; an extract made from biological materials such as viruses, bacteria, plants, fungi, or animal cells; animal tissues; naturally occurring or synthetic compositions; and any combinations thereof.

In some embodiments, the payload comprises a bioactive agent. As used herein, “bioactive agents” refer to synthetic or naturally occurring materials that that have a biological effect on a biological cell, tissue or organ. In some embodiments, the bioactive agent can be a biological material, for example, extracellular matrix materials such as fibronectin, vitronection, and laminin, cytokines, growth factors and differentiation factors, nucleic acids, proteins, peptides, antibodies, and cells. In some embodiments, suitable bioactive agents include, but are not limited to, therapeutic agents. As used herein, the term “therapeutic agent” can refer to a substance used in the diagnosis, treatment, or prevention of a disease. Any therapeutic agent known to those of ordinary skill in the art to be of benefit in the diagnosis, treatment or prevention of a disease is contemplated as a therapeutic agent in the context of the present invention. Therapeutic agents include pharmaceutically active compounds, hormones, growth factors, enzymes, DNA, plasmid DNA, RNA, siRNA, antisense oligonucleotides, modified RNA, viruses, proteins, lipids, pro-inflammatory molecules, polyclonal antibodies, monoclonal antibodies, antibiotics, anti-inflammatory agents, anti-sense nucleotides and transforming nucleic acids or combinations thereof. Any of the therapeutic agents can be combined to the extent such combination is biologically compatible. Examples of therapeutic agents which can be incorporated in the GV-hydrogel, include, but are not limited to, narcotic analgesic drugs; salts of gold; corticosteroids; hormones; antimalarial drugs; indole derivatives; pharmaceuticals for arthritis treatment; antibiotics, including Tetracyclines, Penicillin, Streptomycin and Aureomycin; antihelmintic and canine distemper drugs, applied to domestic animals and large cattle, such, as, for example, phenothiazine; drugs based on sulfur, such, as sulfioxazole; antitumor drugs; pharmaceuticals supervising addictions, such as agents controlling alcohol addiction and agents controlling tobacco addiction; antagonists of drug addiction, such, as methadone; weight controlling drugs; thyroid gland controlling drugs; analgesics; drugs controlling fertilization or contraception hormones; amphetamines; antihypertensive drugs; antiinflammatories agents; antitussives; sedatives; neuromuscular relaxants; antiepileptic drugs; antidepressants; antidisrhythmic drugs; vasodilating drugs; antihypertensive diuretics; antidiabetic agents; anticoagulants; antituberculous agents; antipsychotic agents; hormones and peptides. It is understood that above list is not full and simply represents the wide diversification of therapeutic agents that may be included in the compositions. In some embodiments, therapeutic agent is mitoxantrone, peptide, polyclonal antibody, monoclonal antibody, antigen binding fragment of an antibody, protein (e.g. VEGF) or plasmid DNA.

Therapeutic agents include the herein disclosed categories and specific examples. It is not intended that the category be limited by the specific examples. Those of ordinary skill in the art will recognize also numerous other compounds that fall within the categories and that are useful according to the present disclosure. Examples include a radiosensitizer, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-I-antagonist, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifingal agent, a vaccine, a protein, or a nucleic acid. In a further aspect, the pharmaceutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritic antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetaminophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-i-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as Coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hdyrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

A payload can comprise a micelle, lipsome, or other vesicle-forming lipids. A “liposome” as used herein can refer to a small, spherical vesicle composed of lipids, particularly vesicle-forming lipids capable of spontaneously arranging into lipid bilayer structures in water with its hydrophobic moiety in contact with the interior, hydrophobic region of the bilayer membrane, and its head group moiety oriented toward the exterior, polar surface of the membrane.

A payload can comprise an RNA interfering inhibitory or activating agent, for example a siRNA or a miRNA gene silencer or activator that decreases or increases respectively, the mRNA level of a gene identified herein. The modulating compound results in a decrease or increase, respectively, in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one embodiment, the mRNA levels are decreased or increased respectively by at least about 70%, about 80%, about 90%, about 95%, about 99%, about 100%. As used herein, the term “RNAi” can refer to any type of interfering RNA, including but are not limited to, siRNA, shRNA, endogenous microRNA and artificial microRNA; inhibitory or activating of gene expression. As used herein an “siRNA” can refer to a nucleic acid that forms a double stranded RNA, which double stranded RNA has the ability to reduce or inhibit expression of a gene or target gene when the siRNA is present or expressed in the same cell as the target gene, e.g. the long genes of the brain. The double stranded RNA siRNA can be formed by the complementary strands. In one embodiment, a siRNA can refer to a nucleic acid that can form a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length). In one embodiment, the double stranded siRNA can contain a 3′ and/or 5′ overhang on each strand having a length of about 1, 2, 3, 4, or 5 nucleotides. In one embodiment, the siRNA is capable of promoting inhibitory RNA interference through degradation or specific post-transcriptional gene silencing (PTGS). As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow. The terms “microRNA” or “miRNA” are used interchangeably herein are endogenous RNAs, some of which are known to regulate the expression of protein-coding genes at the posttranscriptional level. Endogenous microRNA are small RNAs naturally present in the genome which are capable of modulating the productive utilization of mRNA. The term artificial microRNA includes any type of RNA sequence, other than endogenous microRNA, which is capable of modulating the productive utilization of mRNA. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

A payload molecule can comprise one or more of (i) a programmable nuclease or a nucleic acid encoding the programmable nuclease, (ii) a targeting molecule or a nucleic acid encoding the targeting molecule, and/or (iii) a donor nucleic acid or a nucleic acid encoding the donor nucleic acid. The targeting molecule can comprise a single guide RNA (sgRNA). The programmable nuclease can comprise a zinc finger nuclease, TAL effector nuclease, meganuclease, MegaTAL, Tev-m TALEN, MegaTev, homing endonuclease, derivatives thereof, or any combination thereof. The programmable nuclease can comprise Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof.

The plurality of payload molecules can comprise a vector (e.g., a viral vector). The vector can comprise an adenovirus vector, an adeno-associated virus vector, an Epstein-Barr virus vector, a Herpes virus vector, an attenuated HIV vector, a lentiviral vector, a retroviral vector, a vaccinia virus vector, or any combination thereof.

Small or Large Organic and/or Inorganic Molecules

The payload molecule can comprise a compound of interest selected from the group consisting of small or large organic or inorganic molecules, carbon-based molecules (e.g., nanotubes, fullerenes, buckeyballs, and the like), metals (e.g., alkali metals, e.g., lithium, sodium, potassium rubidium, caesium, and francium; alkaline earth metals, e.g., beryllium, magnesium, calcium strontium, barium, and radium; transition metals, e.g., zinc, molybdenum, cadmium scandium, titanium, vanadium chromium, manganese, iron cobalt, nickel, copper yttrium, zirconium, niobium technetium, ruthenium, rhodium palladium, silver, hafnium tantalum, tungsten, rhenium osmium, iridium, platinum gold, mercury, rutherfordium, dubnium, seaborgium, bohrium, hassium, and copernicium; post-transition metals, e.g., aluminium, gallium, indium, tin thallium, lead, bismuth; lanthanides, e.g., lanthanum, cerium, praseodymium neodymium, promethium, samarium europium, gadolinium, terbium dysprosium, holmium, erbium thulium, ytterbium, and lutetium; actinides (e.g., actinium, thorium, protactinium uranium, neptunium, plutonium americium, curium, berkelium californium, einsteinium, fermium mendelevium, nobelium, and lawrencium; meitnerium; darmstadtium; roentgenium ununtrium; flerovium ununpentium; livermorium germanium; arsenic; antimony; polonium; and astatine), metal oxides (e.g., titanium dioxide (TiO₂), iron oxides (e.g., Fe₃O₄, Fe₂O₃, and the like), aluminum oxide, antimony tetraoxide, antimony oxide, arsenous oxide, beryllium oxide, bismuth oxide, cadmium oxide, chromic oxide, cobaltic oxide, gallium dioxide, germanium dioxide, hafnium oxide, indium oxide, lead oxide, magnesium oxide, mercuric oxide, molybdenum trioxide, nickel monoxide, niobium pentaoxide, scandium oxide, selenium dioxide, silicon dioxide, silver oxide, tantalum pentaoxide, tellurium dioxide, thallic oxide, thorium oxide, stannic oxide, tungsten trioxide, uranium oxide, vanadium pentoxide, ytrrium oxide, zinc oxide, zirconium dioxide, ceric oxide, dysprosium oxide, erbium oxide, europium oxide, gadolinium oxide, holmium oxide, lanthanum sesquioxide, lutetium oxide, neodymium oxide, samarium oxide, terbium peroxide, thulium oxide, ytterbium oxide, PuO₂, and the like), nanoparticles (e.g., metal nanoparticles, inorganic nanoparticles, gold nanoparticles, silica nanoparticles, calcium carbonate nanoparticles, and the like), imaging agents, contrast agents.

Immunostimulators Agents

The payload molecule can comprise an immunostimulatory agent. As used herein, an immunostimulatory agent is an agent that stimulates an immune response (including enhancing a pre-existing immune response) in a subject to whom it is administered, whether alone or in combination with another agent. Examples include antigens, adjuvants (e.g., TLR ligands such as imiquimod and resiquimod, imidazoquinolines, nucleic acids comprising an unmethylated CpG dinucleotide, monophosphoryl lipid A (MPLA) or other lipopolysaccharide derivatives, single-stranded or double-stranded RNA, flagellin, muramyl dipeptide), cytokines including interleukins (e.g., IL-2, IL-7, IL-15 (or superagonist/mutant forms of these cytokines), IL-12, anti-PD-1, ant-PD-L1, IFN-gamma, IFN-alpha, GM-CSF, FLT3-ligand, etc.), immunostimulatory antibodies (e.g., anti-CTLA-4, anti-CD28, anti-CD3, or single chain/antibody fragments of these molecules), and the like.

Antigens, Adjuvants, Etc.

The payload molecule can comprise an antigen. An antigen may be without limitation a cancer antigen, a self or autoimmune antigen, a microbial antigen, an allergen, or an environmental antigen. The antigen may be peptide, lipid, or carbohydrate in nature, but it is not so limited.

A cancer or tumor antigen is an antigen that is expressed preferentially by cancer cells (e.g., it is expressed at higher levels in cancer cells than on non-cancer cells) and in some instances it is expressed solely by cancer cells. The cancer antigen may be expressed within a cancer cell or on the surface of the cancer cell. The cancer antigen may be MART-1/Melan-A, gp100, adenosine deaminase-binding protein (ADAbp), FAP, cyclophilin b, colorectal associated antigen (CRC)-0017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain, and CD20. The cancer antigen can be MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05). The cancer antigen may be selected from the group consisting of GAGE-1, GAGE-2, GAGE-3, GAGE-4, GAGE-5, GAGE-6, GAGE-7, GAGE-8, GAGE-9. The cancer antigen can be BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100^{Pmel117}, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, lmp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20, and c-erbB-2.

Microbial antigens are antigens derived from microbial species such as without limitation bacterial, viral, fungal, parasitic and mycobacterial species. As such, microbial antigens include bacterial antigens, viral antigens, fungal antigens, parasitic antigens, and mycobacterial antigens. Examples of bacterial, viral, fungal, parasitic and mycobacterial species are provided herein. The microbial antigen may be part of a microbial species or it may be the entire microbe.

An allergen is an agent that can induce an allergic or asthmatic response in a subject. Allergens include without limitation pollens, insect venoms, animal dander dust, fungal spores and drugs (e.g. penicillin).

The payload molecule can comprise an adjuvant. The adjuvant may be without limitation alum (e.g., aluminum hydroxide, aluminum phosphate); saponins purified from the bark of the Q. saponaria tree such as QS21 (a glycolipid that elutes in the 21st peak with IPLC fractionation; Antigenics, Inc., Worcester, Mass.); poly[di(carboxylatophenoxy)phosphazene (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS1312, water-based nanoparticles combined with a soluble immunostimulant, Seppic).

Adjuvants may be TLR ligands. Adjuvants that act through TLR3 include without limitation double-stranded RNA. Adjuvants that act through TLR4 include without limitation derivatives of lipopolysaccharides such as monophosphoryl lipid A (MPLA; Ribi ImmunoChem Research, Inc., Hamilton, Mont.) and muramyl dipeptide (MDP; Ribi) andthreonyl-muramyl dipeptide (t-MDP; Ribi); OM-174 (a glucosamine disaccharide related to lipid A; OM Pharma SA, Meyrin, Switzerland). Adjuvants that act through TLR5 include without limitation flagellin. Adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)). Adjuvants acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages.

Immunoinhibitory Agents and Anti-Inflammatory Agents

The payload molecule can comprise an anti-inflammatory agent and/or immunoinhibitory agent. As used herein, an immunoinhibitory agent is an agent that inhibits an immune response in a subject to whom it is administered, whether alone or in combination with another agent. Examples include steroids, retinoic acid, dexamethasone, cyclophosphamide, anti-CD3 antibody or antibody fragment, and other immunosuppressants. Anti-inflammatory agents are agents that reduce or eliminate inflammation. They include Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Salycilates; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Glucocorticoids; Zomepirac Sodium. One preferred anti-inflammatory agent is aspirin.

Anti-Cancer Agents

The payload molecule can comprise an anti-cancer agent. As used herein, an anti-cancer agent is an agent that at least partially inhibits the development or progression of a cancer, including inhibiting in whole or in part symptoms associated with the cancer even if only for the short term. Several anti-cancer agents can be categorized as DNA damaging agents and these include topoisomerase inhibitors (e.g., etoposide, ramptothecin, topotecan, teniposide, mitoxantrone), DNA alkylating agents (e.g., cisplatin, mechlorethamine, cyclophosphamide, ifosfamide, melphalan, chorambucil, busulfan, thiotepa, carmustine, lomustine, carboplatin, dacarbazine, procarbazine), DNA strand break inducing agents (e.g., bleomycin, doxorubicin, daunorubicin, idarubicin, mitomycin C), anti-microtubule agents (e.g., vincristine, vinblastine), anti-metabolic agents (e.g., cytarabine, methotrexate, hydroxyurea, 5-fluorouracil, floxuridine, 6-thioguanine, 6-mercaptopurine, fludarabine, pentostatin, chlorodeoxyadenosine), anthracyclines, vinca alkaloids, or epipodophyllotoxins.

Examples of anti-cancer agents include without limitation Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Bortezomib (VELCADE); Brequinar Sodium; Bropirimine; Busulfan; Cactinomycin; Calusterone; Caracemide; Carbetimer; Carboplatin (a platinum-containing regimen); Carmustine; Carubicin Hydrochloride; Carzelesin; Cedefingol; Chlorambucil; Cirolemycin; Cisplatin (a platinum-containing regimen); Cladribine; Crisnatol Mesylate; Cyclophosphamide; Cytarabine; Dacarbazine; Dactinomycin; Daunorubicin; Decitabine; Dexormaplatin; Dezaguanine; Diaziquone; Docetaxel (TAXOTERE); Doxorubicin; Droloxifene; Dromostanolone; Duazomycin; Edatrexate; Eflornithine; Elsamitrucin; Enloplatin; Enpromate; Epipropidine; Epirubicin; Erbulozole; Erlotinib (TARCEVA), Esorubicin; Estramustine; Etanidazole; Etoposide; Etoprine; Fadrozole; Fazarabine; Fenretinide; Floxuridine; Fludarabine; 5-Fluorouracil; Fluorocitabine; Fosquidone; Fostriecin; Gefitinib (IRESSA), Gemcitabine; Hydroxyurea; Idarubicin; Ifosfamide; Ilmofosine; Imatinib mesylate (GLEEVAC); Interferon alpha-2a; Interferon alpha-2b; Interferon alpha-ni; Interferon alpha-n3; Interferon beta-I a; Interferon gamma-I b; Iproplatin; Irinotecan; Lanreotide; Lenalidomide (REVLIMID, REVIMID); Letrozole; Leuprolide; Liarozole; Lometerxol; Lomustine; Losoxantrone; Masoprocol; Maytansine; Mechlorethamine; Megestrol; Melengestrol; Melphalan; Menogaril; Mercaptopurine; Methotrexate; Metoprine; Meturedepa; Mitindomide; Mitocarcin; Mitocromin; Mitogillin; Mitomalcin; Mitomycin; Mitosper; Mitotane; Mitoxantrone; Mycophenolic Acid; Nocodazole; Nogalamycin; Ormaplatin; Oxisuran; Paclitaxel; Pemeterxed (ALIMTA), Pegaspargase; Peliomycin; Pentamustine; Pentomone; Peplomycin; Perfosfamide; Pipobroman; Piposulfan; Piritrexim Isethionate; Piroxantrone; Plicamycin; Plomestane; Porfimer; Porfiromycin; Prednimustine; Procarbazine; Puromycin; Pyrazofurin; Riboprine; Rogletimide; Safingol; Semustine; Simtrazene; Sitogluside; Sparfosate; Sparsomycin; Spirogermanium; Spiromustine; Spiroplatin; Streptonigrin; Streptozocin; Sulofenur; Talisomycin; Tamsulosin; Taxol; Taxotere; Tecogalan; Tegafur; Teloxantrone; Temoporfin; Temozolomide (TEMODAR); Teniposide; Teroxirone; Testolactone; Thalidomide (THALOMID) and derivatives thereof, Thiamiprine; Thioguanine; Thiotepa; Tiazofurin; Tirapazamine; Topotecan; Toremifene; Trestolone; Triciribine; Trimeterxate; Triptorelin; Tubulozole; Uracil Mustard; Uredepa; Vapreotide; Verteporfin; Vinblastine; Vincristine; Vindesine; Vinepidine; Vinglycinate; Vinleurosine; Vinorelbine; Vinrosidine; Vinzolidine; Vorozole; Zeniplatin; Zinostatin; Zorubicin.

The anti-cancer agent may be an enzyme inhibitor including without limitation tyrosine kinase inhibitor, a CDK inhibitor, a MAP kinase inhibitor, or an EGFR inhibitor. The tyrosine kinase inhibitor may be without limitation Genistein (4′,5,7-trihydroxyisoflavone), Tyrphostin 25 (3,4,5-trihydroxyphenyl), methylene]-propanedinitrile, Herbimycin A, Daidzein (4′,7-dihydroxyisoflavone), AG-126, trans-1-(3′-carboxy-4′-hydroxyphenyl)-2-(2″,5″-dihydroxy-phenyl)ethane, or HDBA (2-Hydroxy-5-(2,5-Dihydroxybenzylamino)-2-hydroxybenzoic acid. The CDK inhibitor may be without limitation p21, p27, p57, p15, p16, p18, or p19. The MAP kinase inhibitor may be without limitation KY12420 (C₂₃H₂₄O₈), CNI-1493, PD98059, or 4-(4-Fluorophenyl)-2-(4-methylsulfinyl phenyl)-5-(4-pyridyl) 1H-imidazole. The EGFR inhibitor may be without limitation erlotinib (TARCEVA), gefitinib (IRESSA), WH1-P97 (quinazoline derivative), LFM-A12 (leflunomide metabolite analog), ABX-EGF, lapatinib, canertinib, ZD-6474 (ZACTIMA), AEE788, and AG1458.

The anti-cancer agent may be a VEGF inhibitor including without limitation bevacizumab (AVASTIN), ranibizumab (LUCENTIS), pegaptanib (MACUGEN), sorafenib, sunitinib (SUTENT), vatalanib, ZD-6474 (ZACTIMA), anecortave (RETAANE), squalamine lactate, and semaphorin.

The anti-cancer agent may be an antibody or an antibody fragment including without limitation an antibody or an antibody fragment including but not limited to bevacizumab (AVASTIN), trastuzumab (HERCEPTIN), alemtuzumab (CAMPATH, indicated for B cell chronic lymphocytic leukemia), gemtuzumab (MYLOTARG, hP67.6, anti-CD33, indicated for leukemia such as acute myeloid leukemia), rituximab (RITUXAN), tositumomab (BEXXAR, anti-CD20, indicated for B cell malignancy), MDX-210 (bispecific antibody that binds simultaneously to HER-2/neu oncogene protein product and type I Fc Receptors for Immunoglobulin G (IgG) (Fc Gamma RI)), Oregovomab (OVAREX, Indicated for ovarian cancer), edrecolomab (PANOREX), daclizumab (ZENAPAX), palivizumab (SYNAGIS, indicated for respiratory conditions such as RSV infection), ibritumomab tiuxetan (ZEVALIN, indicated for Non-Hodgkin's lymphoma), cetuximab (ERBITUX), MDX-447, MDX-22, MDX-220 (anti-TAG-72), IOR-05, IOR-T6 (anti-CD1), IOR EGF/R3, celogovab (ONCOSCINT OV103), epratuzumab (LYMPHOCIDE), pemtumomab (THERAGYN), and Gliomab-H (indicated for brain cancer, melanoma).

Analgesics

The payload molecule can comprise an analgesic. Analgesics include aspirin, phenybutazone, idomethacin, sulindac, tolmetic, ibuprofen, piroxicam, fenamates, acetaminophen, phenacetin, morphine sulfate, codeine sulfate, meperidine, nalorphine, opioids (e.g., codeine sulfate, fentanyl citrate, hydrocodone bitartrate, loperamide, morphine sulfate, noscapine, norcodeine, normorphine, thebaine, nor-binaltorphimine, buprenorphine, chlomaltrexamine, funaltrexamione, nalbuphine, nalorphine, naloxone, naloxonazine, naltrexone, and naltrindole), procaine, lidocain, tetracaine and dibucaine.

Anti-Infective Agents

The payload molecule can comprise an anti-infective agent. The payload molecule may be an anti-infective agent including without limitation an anti-bacterial agent, an anti-viral agent, an anti-parasitic agent, an anti-fungal agent, and/or an anti-mycobacterial agent.

The payload molecule can comprise an anti-bacterial agent. Anti-bacterial agents may be without limitation β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), other β-lactams (such as imipenem, monobactams), β-lactamase inhibitors, vancomycin, aminoglycosides and spectinomycin, tetracyclines, chloramphenicol, erythromycin, lincomycin, clindamycin, rifampin, metronidazole, polymyxins, sulfonamides and trimethoprim, or quinolines. Other anti-bacterials may be without limitation Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfas alazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; or Zorbamycin. Anti-mycobacterial agents may be without limitation Myambutol (Ethambutol Hydrochloride), Dapsone (4,4′-diaminodiphenylsulfone), Paser Granules (aminosalicylic acid granules), Priftin (rifapentine), Pyrazinamide, Isoniazid, Rifadin (Rifampin), Rifadin IV, Rifamate (Rifampin and Isoniazid), Rifater (Rifampin, Isoniazid, and Pyrazinamide), Streptomycin Sulfate or Trecator-SC (Ethionamide).

Anti-fungal agents may be without limitation imidazoles and triazoles, polyene macrolide antibiotics, griseofulvin, amphotericin B, and flucytosine. Antiparasites include heavy metals, antimalarial quinolines, folate antagonists, nitroimidazoles, benzimidazoles, avermectins, praxiquantel, ornithine decarboxylase inhibitors, phenols (e.g., bithionol, niclosamide); synthetic alkaloid (e.g., dehydroemetine); piperazines (e.g., diethylcarbamazine); acetanilide (e.g., diloxanide furonate); halogenated quinolines (e.g., iodoquinol (diiodohydroxyquin)); nitrofurans (e.g., nifurtimox); diamidines (e.g., pentamidine); tetrahydropyrimidine (e.g., pyrantel pamoate); or sulfated naphthylamine (e.g., suramin). Other anti-infective agents may be without limitation Difloxacin Hydrochloride; Lauryl Isoquinolinium Bromide; Moxalactam Disodium; Ornidazole; Pentisomicin; Sarafloxacin Hydrochloride; Protease inhibitors of HIV and other retroviruses; Integrase Inhibitors of HIV and other retroviruses; Cefaclor (Ceclor); Acyclovir (Zovirax); Norfloxacin (Noroxin); Cefoxitin (Mefoxin); Cefuroxime axetil (Ceftin); Ciprofloxacin (Cipro); Aminacrine Hydrochloride; Benzethonium Chloride: Bithionolate Sodium; Bromchlorenone; Carbamide Peroxide; Cetalkonium Chloride; Cetylpyridinium Chloride: Chlorhexidine Hydrochloride; Clioquinol; Domiphen Bromide; Fenticlor; Fludazonium Chloride; Fuchsin, Basic; Furazolidone; Gentian Violet; Halquinols; Hexachlorophene: Hydrogen Peroxide; Ichthammol; Imidecyl Iodine; Iodine; Isopropyl Alcohol; Mafenide Acetate; Meralein Sodium; Mercufenol Chloride; Mercury, Ammoniated; Methylbenzethonium Chloride; Nitrofurazone; Nitromersol; Octenidine Hydrochloride; Oxychlorosene; Oxychlorosene Sodium; Parachlorophenol, Camphorated; Potassium Permanganate; Povidone-Iodine; Sepazonium Chloride; Silver Nitrate; Sulfadiazine, Silver; Symclosene; Thimerfonate Sodium; Thimerosal; or Troclosene Potassium.

Anti-viral agents may be without limitation amantidine and rimantadine, ribivarin, acyclovir, vidarabine, trifluorothymidine, ganciclovir, zidovudine, retinovir, and interferons. Anti-viral agents may be without limitation further include Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; Zinviroxime or integrase inhibitors.

Cardiovascular Agents

The payload molecule can comprise a cardiovascular agent, such as, for example, an antithrombotic or thrombolytic agent or fibrinolytic agent selected from the group consisting of anticoagulants, anticoagulant antagonists, antiplatelet agents, thrombolytic agents, thrombolytic agent antagonists, and any combinations thereof.

In some embodiments, the payload molecule is thrombogenic agent selected from thrombolytic agent antagonists, anticoagulant antagonists, pro-coagulant enzymes, pro-coagulant proteins, and any combinations thereof. Some exemplary thrombogenic agents include, but are not limited to, protamines, vitamin K1, amiocaproic acid (amicar), tranexamic acid (amstat), anagrelide, argatroban, cilstazol, daltroban, defibrotide, enoxaparin, fraxiparine, indobufen, lamoparan, ozagrel, picotamide, plafibride, tedelparin, ticlopidine, triflusal, collagen, and collagen-coated particles.

In some embodiments, the payload molecule is a thrombolytic agent. As used herein, the term “thrombolytic agent” can refer to any agent capable of inducing reperfusion by dissolving, dislodging or otherwise breaking up a clot, e.g., by either dissolving a fibrin-platelet clot, or inhibiting the formation of such a clot. Reperfusion occurs when the clot is dissolved and blood flow is restored. Exemplary thrombolytic agents include, but are not limited to, tissue-type plasminogen activator (t-PA), streptokinase (SK), prourokinase, urokinase (uPA), alteplase (also known as Activase®, Genentech, Inc.), reteplase (also known as r-PA or Retavase®, Centocor, Inc.), tenecteplase (also known as TNK™, Genentech, Inc.), Streptase® (AstraZeneca, LP), lanoteplase (Bristol-Myers Squibb Company), monteplase (Eisai Company, Ltd.), saruplase (also known as r-scu-PA and Rescupase™, Grunenthal GmbH, Corp.), staphylokinase, and anisoylated plasminogen-streptokinase activator complex (also known as APSAC, Anistreplase and Eminase®, SmithKline Beecham Corp.). Thrombolytic agents also include other genetically engineered plasminogen activators. The invention can additionally employ hybrids, physiologically active fragments or mutant forms of the above thrombolytic agents. The term “tissue-type plasminogen activator” as used herein is intended to include such hybrids, fragments and mutants, as well as both naturally derived and recombinantly derived tissue-type plasminogen activator. The term “anticoagulant” is meant to refer to any agent capable of prolonging the prothrombin and partial thromboplastin time tests and reducing the levels of prothrombin and factors VII, IX and X. Anticoagulants typically include cormarin derivatives and heparin as well as aspirin, which may also be referred to as an antiplatelet agent.

In some embodiments, the payload molecule is an agent known in the art for treatment of inflammation or inflammation associated disorders, or infections. Exemplary anti-inflammatory agents include, but are not limited to, non-steroidal anti-inflammatory drugs (NSAIDs-such as aspirin, ibuprofen, or naproxen), coricosteroids (such as presnisone), anti-malarial medication (such as hydrochloroquine), methotrexrate, sulfasalazine, leflunomide, anti-TNF medications, cyclophosphamise, mycophenolate, dexamethasone, rosiglitazone, prednisolone, corticosterone, budesonide, estrogen, estrodiol, sulfasalazine, fenfibrate, provastatin, simvastatin, proglitazone, acetylsalicylic acid, mycophenolic acid, mesalamine, hydroxyurea, and analogs, derivatives, prodrugs, and pharmaceutically acceptable salts thereof.

In some embodiments, the payload molecule is a vasodilator. A vasodilator can be alpha-adrenoceptor antagonists (alpha-blockers), agiotensin converting enzyme (ACE) inhibitors, angiotensin receptor blockers (ARBs), beta2-adrenoceptor agonists (β2-agonists), calcium-channel blockers (CCBs), centrally acting sympatholytics, direct acting vasodilators, endothelin receptor antagonists, ganglionic blockers, nitrodilators, phosphodiesterase inhibitors, potassium-channel openers, renin inhibitors, and any combinations thereof. Exemplary vasodilator include, but are not limited to, prazosin, terazosin, doxazosin, trimazosin, phentolamine, phenoxybenzamine, benazepril, captopril, enalapril, fosinopril, lisinopril, moexipril, quinapril, ramipril, candesartan, eprosartan, irbesartan, losartan, olmesartan, telmisartan, valsartan, Epinephrine, Norepinephrine, Dopamine, Dobutamine, Isoproterenol, amlodipine, felodipine, isradipine, nicardipine, nifedipine, nimodipine, nitrendipine, clonidine, guanabenz, guanfacine, α-methyldopa, hydralazine, Bosentan, trimethaphan camsylate, isosorbide dinitrate, isosorbide mononitrate, nitroglycerin, erythrityl tetranitrate, pentaerythritol tetranitrate, sodium nitroprusside, milrinone, inaminone (formerly aminone), cilostazol, sildenafil, tadalafil, minoxidil, aliskiren, and analogs, derivatives, prodrugs, and pharmaceutically acceptable salts thereof.

In some embodiments, the payload molecule is a vasoconstrictor. As used herein, the term “vasoconstrictor” can refer to compounds or molecules that narrow blood vessels and thereby maintain or increase blood pressure, and/or decrease blood flow. There are many disorders that can benefit from treatment using a vasoconstrictor. For example, redness of the skin (e.g., erythema or cuperose), which typically involves dilated blood vessels, benefit from treatment with a vasoconstrictor, which shrinks the capillaries thereby decreasing the untoward redness. Other descriptive names of the vasoconstrictor group include vasoactive agonists, vasopressor agents and vasoconstrictor drugs. Certain vasoconstrictors act on specific receptors, such as vasopres sin receptors or adrenoreceptors. Exemplary vasoconstrictors include, but are not limited to, alpha-adrenoreceptor agonists, chatecolamines, vasopressin, vasopressin receptor modualors, calcium channel agonists, and other endogenous or exogenous vasoconstrictors.

In some embodiments, the vasoconstrictor can be aluminum sulfate, amidephrine, amphetamines, angiotensin, antihistamines, argipres sin, bismuth subgallate, cafaminol, caffeine, catecholamines, cyclopentamine, deoxyepinephrine, dopamine, ephedrine, epinephrine, felypressin, indanazoline, isoproterenol, lisergic acid diethylamine, lypressin (LVP), lysergic acid, mephedrone, methoxamine, methylphenidate, metizoline, metraminol, midodrine, naphazoline, nordefrin, norepinephrine, octodrine, ornipressin, oxymethazoline, phenylefhanolamine, phenylephrine, phenylisopropylamines, phenylpropanolamine, phenypres sin, propylhexedrine, pseudoephedrine, psilocybin, tetrahydralazine, tetrahydrozoline, tetrahydrozoline hydrochloride, tetrahydrozoline hydrochloride with zinc sulfate, tramazoline, tuaminoheptane, tymazoline, vasopressin, vasotocin, xylometazoline, zinc oxide, or the like.

In some embodiments, the vasoactive agent is a substance derived or extracted from a herbal source, e.g., Ephedra sinica (ma huang), Polygonum bistorta (bistort root), Hamamelis virginiana (witch hazel), Hydrastis canadensis (goldenseal), Lycopus virginicus (bugleweed), Aspidosperma quebracho (quebracho bianco), Cytisus scoparius (scotch broom), cypress and salts, isomers, analogs and derivatives thereof.

In some embodiments, the payload molecule can be aspirin, wafarin (coumadin), acenocoumarol, ancrod, anisindione, bromindione, clorindione, coumetarol, cyclocumarol, dextran, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol, dipyridamole (persantin), sulfinpyranone (anturane), ticlopidine (ticlid), tissue plasminogen activator (activase), plasmin, pro-urokinase, urokinase (abbokinase) streptokinase (streptase), and anistreplase/APSAC (eminase), and analogs, derivatives, prodrugs, and pharmaceutically acceptable salts thereof.

Hormones

In some embodiments, the payload molecule is a hormone. Hormones include estrogens (e.g., estradiol, estrone, estriol, diethylstibestrol, quinestrol, chlorotrianisene, ethinyl estradiol, mestranol), anti-estrogens (e.g., clomiphene, tamoxifen), progestins (e.g., medroxyprogesterone, norethindrone, hydroxyprogesterone, norgestrel), antiprogestin (mifepristone), androgens (e.g., testosterone cypionate, fluoxymesterone, danazol, testolactone), anti-androgens (e.g., cyproterone acetate, flutamide), thyroid hormones (e.g., triiodothyronne, thyroxine, propylthiouracil, methimazole, and iodixode), and pituitary hormones (e.g., corticotropin, sumutotropin, oxytocin, and vasopressin). Hormones are commonly employed in hormone replacement therapy and/or for purposes of birth control. Steroid hormones, such as prednisone, are also used as immunosuppressants and anti-inflammatories.

Muscle relaxants include mephenesin, methocarbomal, cyclobenzaprine hydrochloride, trihexylphenidyl hydrochloride, levodopa/carbidopa, and biperiden. Anti-spasmodics include atropine, scopolamine, oxyphenonium, and papaverine. Ophthalmic agents include sodium fluorescein, rose bengal, methacholine, adrenaline, cocaine, atropine, alpha-chymotrypsin, hyaluronidase, betaxalol, pilocarpine, timolol, timolol salts, and combinations thereof.

Growth Trophic Factors

In some embodiments, the payload molecule is atrophic factor. Trophic factors are factors whose continued presence improves the viability or longevity of a cell. Trophic factors include, Without limitation, platelet-derived growth factor (PDGP), neutrophil-activating protein, monocyte chemoattractant protein, macrophage-inflammatory protein, platelet factor, platelet basic protein, and melanoma growth stimulating activity; epidermal growth factor, transforming growth factor (alpha), fibroblast growth factor, platelet-derived endothelial cell growth factor, insulin-like growth factor, glial derived growth neurotrophic factor, ciliary neurotrophic factor, nerve growth factor, bone growth/cartilage-inducing factor (alpha and beta), bone morphogenetic proteins, interleukins (e.g., interleukin inhibitors or interleukin receptors, including interleukin 1 through interleukin 10), interferons (e.g., interferon alpha, beta and gamma), hematopoietic factors, including erythropoietin, granulocyte colony stimulating factor, macrophage colony stimulating factor and granulocyte-macrophage colony stimulating factor; tumor necrosis factors, and transforming growth factors (beta), including beta-1, beta-2, beta-3, inhibin, and activin.

In some embodiments, the payload molecule is an osteogenic protein. In some embodiments, the payload molecule is desirably selected from the family of proteins known as the transforming growth factors beta (TGF-β) superfamily of proteins, which includes the activins, inhibins and bone morphogenetic proteins (BMPs). In some embodiments, the payload molecule is at least one protein selected from the subclass of proteins known generally as BMPs, which have been disclosed to have osteogenic activity, and other growth and differentiation type activities. Subsets of BMPs which can be used include BMP-2, BMP-3, BMP-3b, BMP-4, BMP-5, BMP-6, BMP-7, BMP-8, BMP-9, BMP-10, BMP-11, BMP-12, BMP-13, BMP-14, BMP-15, BMP-16, BMP-17, and BMP18. Other osteogenic agents known in the art can also be used, such as teriparatide (FORTEO™) CHRYSALIN®, prostaglandin E2, or LIM protein, among others.

Imaging Agents, Contrast Agents, and Dignostic Agents

The payload can comprise a contrast agent, an imaging agent, a contrast agent, and/or a diagnostic agent. The methods disclosed herein can comprise, in some embodiments, determining the presence and/or progression of a disease or disorder using the contrast agent, the imaging agent and/or the diagnostic agent.

As used herein, the term “imaging agent” can refer to an element or functional group in a molecule that allows for the detection, imaging, and/or monitoring of the presence and/or progression of a condition(s), pathological disorder(s), and/or disease(s). The imaging agent may be an echogenic substance (either liquid or gas), non-metallic isotope, an optical reporter, a boron neutron absorber, a paramagnetic metal ion, a ferromagnetic metal, a gamma-emitting radioisotope, a positron-emitting radioisotope, or an x-ray absorber. As used herein the term “contrast agent” can refer to any molecule that changes the optical properties of tissue or organ containing the molecule. Optical properties that can be changed include, but are not limited to, absorbance, reflectance, fluorescence, birefringence, optical scattering and the like. In some embodiments, the payload is a diagnostic agent. As used herein, “diagnostic agent” comprises any agent that can be used in the diagnosis of a disease in an individual. Non-limiting examples include imaging agents such as radioisotopes, dyes, pigments and fluorescent molecules (such as luciferase, and fluorescein) and heavy metals (such as gadolinium).

Suitable optical reporters include, but are not limited to, fluorescent reporters and chemiluminescent groups. A wide variety of fluorescent reporter dyes are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound. Suitable fluorescent reporters include xanthene dyes, such as fluorescein or rhodamine dyes, including, but not limited to, Alexa Fluor® dyes (InvitrogenCorp.; Carlsbad, Calif.), fluorescein, fluorescein isothiocyanate (FITC), Oregon Green™, rhodamine, Texas red, tetrarhodamine isothiocynate (TRITC), 5-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N,N′-tetramefhyl-6-carboxyrhodamine (TAMRA), 6-carboxy-X-rhodamine (ROX). Suitable fluorescent reporters also include the naphthylamine dyes that have an amino group in the alpha or beta position. For example, naphthylamino compounds include 1-dimethylamino-naphthyl-5-sulfonate, 1-anilino-8-naphthalene sulfonate, 2-p-toluidinyl-6-naphthalene sulfonate, and 5-(2′-aminoethyl)aminonaphthalene-1-sulfonic acid (EDANS). Other fluorescent reporter dyes include coumarins, such as 3-phenyl-7-isocyanatocoumarin; acridines, such as 9-isothiocyanatoacridine and acridine orange; N-(p(2-benzoxazolyl)phenyl)maleimide; cyanines, such as Cy2, indodicarbocyanine 3 (Cy3), indodicarbocyanine 5 (Cy5), indodicarbocyanine 5.5 (Cy5.5), 3-(-carboxy-pentyl)-3′ethyl-5,5′-dimethyloxacarbocyanine (CyA); 1H,5H,11H, 15H-Xantheno[2,3,4-ij:5,6,7-i′j′]diquinolizin-18-ium, 9-[2(or 4)-[[[6-[2,5-dioxo-1-pyrrolidinyl)oxy]-6-oxohexyl]amino]sulfonyl]-4(or 2)-sulfophenyl]-2,3,6,7,12,13,16,17octahydro-inner salt (TR or Texas Red); BODIPY™ dyes; benzoxadiazoles; stilbenes; pyrenes; and the like. Many suitable forms of these fluorescent compounds are available and can be used.

Suitable echogenic gases include, but are not limited to, a sulfur hexafluoride or perfluorocarbon gas, such as perfluoromethane, perfluoroethane, perfluoropropane, perfluorobutane, perfluorocyclobutane, perfluropentane, or perfluorohexane.

Suitable non-metallic isotopes include, but are not limited to, ¹¹C, ¹⁴C, ¹³N, ¹⁸F, ¹²³I, ¹²⁴I, and ¹²⁵I. Suitable radioisotopes include, but are not limited to, ⁹⁹mTc, ⁹⁵Tc, ¹¹¹In, ⁶²Cu, ⁶⁴Cu, Ga, ⁶⁸Ga, and ¹⁵³Gd. Suitable paramagnetic metal ions include, but are not limited to, Gd(III), Dy(III), Fe(III), and Mn(II). Suitable X-ray absorbers include, but are not limited to, Re, Sm, Ho, Lu, Pm, Y, Bi, Pd, Gd, La, Au, Au, Yb, Dy, Cu, Rh, Ag, and Ir. In some embodiments, the radionuclide is bound to a chelating agent or chelating agent-linker attached to the cryogel. Suitable radionuclides for direct conjugation include, without limitation, ¹⁸F, ¹²⁴I, ¹²⁵, ¹³¹I, and mixtures thereof. Suitable radionuclides for use with a chelating agent include, without limitation, ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ^{117m}Sn, ¹⁴⁹Pm, ¹⁵³Sm, ¹⁶⁶H o, ¹⁷⁷Lu, ¹⁸⁶Re, ¹⁸⁸Re, ²¹¹At, and mixtures thereof. Suitable chelating agents include, but are not limited to, DOTA, BAD, TETA, DTPA, EDTA, NTA, HDTA, their phosphonate analogs, and mixtures thereof.

### Methods of Payload Delivery and Treatment

Disclosed herein include methods for the spatial and temporal delivery of payload molecules to a target site of a subject. In some embodiments, the method comprises: administering a hydrogel composition disclosed herein to a subject; applying a first ultrasonic (US) pulse (or set of pulses) to a target site of the subject; detecting the presence of the hydrogel composition at the target site; and applying a second US pulse (or set of pulses) to the target site of the subject, wherein the second US pulse (or set of pulses) induces the release of payload molecules from the hydrogel composition, thereby delivering payload molecules to the target site.

Disclosed herein include methods of treating a disease or disorder. In some embodiments, the method comprises: administering a hydrogel composition disclosed herein to a subject suffering from a disease or disorder; applying a first ultrasonic (US) pulse (or set of pulses) to a target site of the subject; detecting the presence of the hydrogel composition at the target site; and applying a second US pulse (or set of pulses) to the target site of the subject, wherein the second US pulse (or set of pulses) induces the release of payload molecules from the hydrogel composition, thereby delivering payload molecules to the target site and treat the subject of the disease or disorder.

The methods of treating a disease or disorder and/or the methods of spatial and temporal delivery of payload molecules to a target site of a subject can comprise: administering a hydrogel composition disclosed herein to a subject; applying imaging ultrasound (e.g., a first ultrasonic (US) pulse(s)) to a target site of the subject; detecting the presence of the hydrogel composition at the target site; and applying collapsing ultrasound (e.g., a second US pulse(s)) to the target site of the subject, wherein the collapsing ultrasound induces the release of payload molecules from the hydrogel composition, thereby delivering payload molecules to the target site and/or treating the subject of the disease or disorder. The collapsing ultrasound and imaging ultrasound can be selected to have a collapsing pressure and an imaging pressure amplitude based on the acoustic collapse pressure profile of the GV type embedded within the hydrogel composition.

Disclosed herein include methods for the spatial and temporal delivery of payload molecules to a target site of a subject. In some embodiments, the method comprises: administering a hydrogel composition disclosed herein to a subject; and applying one or more ultrasonic (US) pulses to a target site of the subject, wherein the one or more US pulses induce the release of payload molecules from the hydrogel composition, thereby delivering payload molecules to the target site. Disclosed herein include methods of treating a disease or disorder. In some embodiments, the method comprises: administering a hydrogel composition disclosed herein to a subject; and applying one or more ultrasonic (US) pulses to a target site of the subject, wherein the one or more US pulses induce the release of payload molecules from the hydrogel composition, thereby delivering payload molecules to the target site and treat the subject of the disease or disorder. Applying one or more US pulses can comprise applying second US pulse(s). In some embodiments, the second US pulse(s) induces the release of payload molecules from the hydrogel composition. The method can comprise detecting the presence of the hydrogel composition at the target site prior to inducing the release of payload molecules from the hydrogel composition. Applying one or more US pulses can comprise applying fifth US pulse(s). The method can comprise applying first US pulse(s), applying third US pulse(s), applying fourth US pulse(s), and/or applying sixth US pulse(s) to the target site.

The one or more particles can comprise a mixture of particles. The mixture of particles (e.g., hydrogels) can be heterogenous. The mixture of particles can comprise between about 1 and about 100 particles with different compositions (e.g., different polymer matrix, payload, GV, or any combination thereof). The mixture of particles can comprise one or more first particles and one or more second particles. There are provided, in some embodiments, hydrogel compositions comprising a mixture of particles comprising different types of payload molecules and/or gas vesicles. The polymer matrix of the first particles and second particles can be the same or different. The ratio of first particles to second particles can vary. The one or more particles can comprise one or more first particles and one or more second particles. The one or more first particles can comprise a plurality of payload molecules and a plurality of gas vesicles. The one or more second particles comprise a plurality of secondary payload molecules and a plurality of secondary gas vesicles. The gas vesicles and secondary gas vesicles can have different collapse pressure profiles. In some embodiments of the methods provided herein, the payload molecules of the one or more first particles are delivered to a first target site, and the secondary payload molecules of the one or more second particles are delivered to a second target site. The first target site and the second target site can be the same or different areas of the subject. In some embodiments, the payload of the first particles is released at the first target site and the payload of the second particles is not. In some embodiments, the payload of the second particles is released at the second target site and the payload of the first particles is not. In some embodiments, the payload of the first particles is released prior to the payload of the second particles.

The method can comprise applying collapsing ultrasound (e.g., second US pulse(s)) that causes the collapse of the gas vesicles within the one or more first particles (and thereby induce release of embedded payload molecules) but does not cause the collapse of gas vesicles (e.g., secondary gas vesicles) situated within the one or more second particles. The method can further comprise applying collapsing ultrasound (e.g., fifth US pulse(s)) that cause the collapse of the secondary gas vesicles within the one or more second particles (and thereby induce release of embedded secondary payload molecules). The method can comprise detecting the presence of the one or more first particles at a target site (e.g., a first target site) using the gas vesicles as contrast agents as disclosed herein (e.g., imaging ultrasound, detecting scattering of first US pulse(s) and/or third US pulse(s)). The method can comprise detecting the presence of the one or more second particles at a target site using the secondary gas vesicles as contrast agents as disclosed herein (e.g., imaging ultrasound, detecting scattering of fourth US pulse(s) and/or sixth US pulse(s)). The method can comprise detecting the presence of the one or more first particles at a target site (e.g., a second target site) in the subject using the gas vesicles as contrast agents as disclosed herein and/or detecting the presence of the one or more first particles at a target site in the subject using the secondary gas vesicles as contrast agents as disclosed herein. Confirming the delivery of payload molecules to a target site (e.g., a first target site) can comprise detecting a difference in the images derived from the first US pulse(s) and third US pulse(s). Confirming the delivery of secondary payload molecules to a target site (e.g., a second target site) can comprise detecting a difference in the images derived from the fourth US pulse(s) and sixth US pulse(s). In some embodiments, imaging US is not performed. In some such embodiments, only collapsing US is performed. For example, in some embodiments, collapsing US can be applied to a general region of anatomy. In some embodiments, the first US pulse(s) and the third US pulse(s) have the same parameters. In some embodiments, the fourth US pulse(s) and the sixth US pulse(s) have the same parameters.

The basic physics of sound waves enables ultrasound to visualize biological tissues with high spatial and temporal resolution. This capability has been enhanced by the development of acoustic biomolecules proteins with physical properties enabling them to scatter sound. The first acoustic biomolecules developed as contrast agents in ultrasound imaging, analogous to GFPs used in optical imaging, were based on a unique class of air-filled protein nanostructures called gas vesicles (GVs). The advancement of GVs has made it possible to use ultrasound to visualize the functions of cells deep inside tissues.

The term “ultrasound” can refer to sound with frequencies higher than the audible limits of human beings, typically over 20 kHz. Ultrasound devices typically can range up to the gigahertz range of frequencies, with most medical ultrasound devices operating in the 0.2 to 18 MHz range. The amplitude of the waves relates to the intensity of the ultrasound, which in turn relates to the pressure created by the ultrasound waves. Applying ultrasound can be accomplished, for example, by sending strong, short electrical pulses to a piezoelectric transducer directed at the target. Ultrasound can be applied as a continuous wave, or as wave pulses as will be understood by a person skilled in focused ultrasound. U.S. Patent Application Publication No. 2020/0237346 describes methods comprising the application of a step function increase in acoustic pressure during ultrasound imaging using gas vesicle contrast, along with capturing successive frames of ultrasound imaging and extracting time-series vectors for pixels of the frames, the content of which is hereby expressly incorporated by reference in its entirety. In some embodiments, the first, second, third, fourth, fifth, and/or sixth US pulse(s) each comprise a set of pulses.

Focused ultrasound (“FUS”) can refer to the technology that uses ultrasound energy to target specific areas of a subject, such as a specific area of a brain or body. FUS focuses acoustic waves by employing concave transducers that usually have a single geometric focus, or an array of ultrasound transducer elements which are actuated in a spatiotemporal pattern such as to produce one or more focal zones. At this focus or foci most of the power is delivered during sonication in order to induce mechanical effects, thermal effects, or both. The frequencies used for focused ultrasound are in the range of 200 KHz to 8000 KHz.

As used herein, the term “harmonic signal” or “harmonic frequency” can refer to a frequency in a periodic waveform that is an integer multiple of the frequency of the fundamental signal. In addition, this term encompasses sub-harmonic signals, which are signals with a frequency equal to an integral submultiple of the frequency of the fundamental signal. In ultrasound imaging, the transmitted pulse is typically centered around a fundamental frequency, and received signals may be processed to isolate signals centered around the fundamental frequency or one or more harmonic frequencies.

The term “fundamental signal” or “fundamental wave” can refer to the primary frequency of the transmitted ultrasound pulse. All GVs can backscatter ultrasound at the fundamental frequency, allowing their detection by ultrasound.

The term “non-linear signal” can refer to a signal that does not obey superposition and scaling properties, with regards to the input. The term “linear signal” can refer to a signal that does obey those properties. One example of non-linearity is the production of harmonic signals in response to ultrasound excitation at a certain fundamental frequency. Another example is a non-linear response to acoustic pressure. One embodiment of such a non-linearity is the acoustic collapse profile of GVs, in which there is a non-linear relationship between the applied pressure and the disappearance of subsequent ultrasound contrast from the GVs as they collapse. Another example of a non-linear signal that does not involve the destruction of GVs, is the increase in both fundamental and harmonic signals with increasing pressure of the transmitted imaging pulse, wherein certain GVs exhibit a super-linear relationship between these signals and the pulse pressure.

The term “applying ultrasound” shall be given its ordinary meaning, and shall also refer to sending ultrasound-range acoustic energy to a target. The sound energy produced by the piezoelectric transducer can be focused by beamforming, through transducer shape, lensing, or use of control pulses. The soundwave formed is transmitted to the body, then partially reflected or scattered by structures within a body; larger structures typically reflecting, and smaller structures typically scattering. The return sound energy reflected/scattered to the transducer vibrates the transducer and turns the return sound energy into electrical signals to be analyzed for imaging. The frequency and pressure of the input sound energy can be controlled and are selected based on the needs of the particular imaging/delivery task and, in some methods described herein, collapsing GVs (thereby inducing hydrogel compositions provided herein to release embedded payload molecules at a target site). To create images, particularly 2D and 3D imaging, scanning techniques can be used where the ultrasound energy is applied in lines or slices which are composited into an image.

In some embodiments, detecting the presence of the hydrogel composition at the target site can comprise detecting scattering of the first US pulse(s) (e.g., imaging ultrasound) by the gas vesicles. The method can comprise: confirming the delivery of payload molecules at the target site. Confirming the delivery of payload molecules can comprise detecting reduced scattering of the second US pulse(s) (e.g., collapsing ultrasound) by the gas vesicles. The gas vesicles can be capable of acting as a contrast agent at the first US pulse(s) (e.g., imaging ultrasound) but not at the second US pulse(s) (e.g., collapsing ultrasound). The first US pulse(s) (e.g., imaging ultrasound) can comprise a pressure value less than the first selectable collapse pressure value.

The second US pulse(s) (e.g., collapsing ultrasound) can comprise a pressure value equal to or higher than the first selectable collapse pressure value. In some embodiments, the second US pulse(s) (e.g., collapsing ultrasound) induces gas vesicle collapse. In some embodiments, the second US pulse(s) (e.g., collapsing ultrasound) increases the diffusivity of the hydrogel material by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values). In some embodiments, the second US pulse(s) (e.g., collapsing ultrasound) increases the porosity of the hydrogel composition by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values). In some embodiments, the second US pulse(s) (e.g., collapsing ultrasound) increases the rate of payload molecule release from the hydrogel composition by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values).

The gas vesicles can be capable of acting as a contrast agent at the first US pulse(s) (e.g., imaging ultrasound) but not at the second US pulse(s) (e.g., collapsing ultrasound).

In some embodiments of the methods disclosed herein comprise stable cavitation of nanobubbles liberated from the GVs following collapse and/or inertial cavitation of liberated nanobubbles. Compositions and methods related to stable and/or inertial cavitation of nanobubbles liberated from the GVs following collapse are disclosed in Bar-Zion, Avinoam, et al. (“Acoustically detonated biomolecules for genetically encodable inertial cavitation.” (2019) bioRxiv 620567; https://doi.org/10.1101/620567), hereby incorporated by reference in its entirety. In some embodiments, the gas vesicle collapse results in the release of a nanoscale air bubble. In some embodiments, the released nanoscale air bubble undergoes cavitation and is converted into a micron-scale air bubble. The second US pulse(s) (e.g., collapsing ultrasound) can be capable of inducing cavitation. The cavitation can comprise cavitation of the gas vesicles and/or bubbles created by gas vesicle collapse. The gas vesicles can be capable as acting as the nuclei for the formation and/or cavitation of bubbles. The cavitation can comprise stable cavitation. The cavitation can comprise inertial cavitation. In some embodiments, the cavitation triggers the degradation of the hydrogel composition. In some embodiments, the cavitation induces the release of payload molecules from the hydrogel composition. In some embodiments, the cavitation exerts mechanical forces and/or thermal forces on the hydrogel composition, thereby inducing the release of payload molecules. The target site can comprise target cells. In some embodiments, the cavitation exerts mechanical forces and/or thermal forces on target cells proximate to the hydrogel composition, thereby enhancing uptake of payload molecules by said target cells. In some embodiments, said mechanical forces and/or thermal forces reduce the membrane permeability of target cells proximate to the hydrogel composition. The peak positive pressure of the second US pulse(s) (e.g., collapsing ultrasound) can be equal to or higher than an initial collapse pressure of the gas vesicles, thereby collapsing the gas vesicles. The peak negative pressure of the second US pulse(s) (e.g., collapsing ultrasound) can be below the critical cavitation pressure of the gas vesicles.

In some embodiments, at least about 5% (e.g., at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or a number or a range between any two of these values) of the plurality of payload molecules are released at the target site. In some embodiments, less than about 95% (95%, 90%, 85%, 80%, 75%, 70%, 65%, 60%, 55%, 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, or a number or a range between any two of these values) of the plurality of payload molecules are released at a location other than the target site.

In some embodiments, at least about 5% (e.g., at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or a number or a range between any two of these values) of the plurality of payload molecules are released from the hydrogel composition within about 1 ns, about 10 ns, about 100 ns, about 1 ms, about 10 ms, about 100 ms, about 1 s, or a number or a range between any two of these values) after the second US pulse(s).

In some embodiments, at least about 5%, (e.g., at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or a number or a range between any two of these values) of the plurality of payload molecules are released from the hydrogel composition within at least about 1 nm, (e.g., about 1 nm, about 10 nm, about 100 nm, about 1 μm, about 10 μm, about 100 μm, about 1 mm, about 10 mm, about 100 mm, or a number or a range between any two of these values) of the location of the hydrogel composition at the time of the second US pulse(s).

The ratio of the concentration of payload molecules at the subject's target site to the concentration of payload molecules in subject's blood, serum, or plasma can be about 2:1 to about 3000:1, about 2:1 to about 2000:1, about 2:1 to about 1000:1, or about 2:1 to about 600:1. In some embodiments, the ratio of the concentration of payload molecules at the subject's target site to the concentration of payload molecules in subject's blood, serum, or plasma can be, or be about, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, 10000:1, or a number or a range between any two of the values. In some embodiments, the ratio of the concentration of payload molecules at the subject's target site to the concentration of payload molecules in subject's blood, serum, or plasma can be at least, or be at most, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, or 10000:1.

In some embodiments, multiplexed imaging and payload delivery methods are provided. The term “multiplex” can refer to the presence of two or more distinct GVPS types, each of which exhibits an acoustic collapse pressure profile distinct from one another, in the hydrogel composition. The two or more distinct GVPSs can be derived from different organisms or variants of GVPSs from the same or different organisms (e.g., archaea).

In multiplexing methods herein described, both the collapsing pressure of the collapsing ultrasound and the imaging pressure of the imaging ultrasound are selected based on the acoustic collapse pressure profiles of the GVPS types (e.g., peak acoustic pressure) to selectively collapse one GVPS type over the other GVPS types. The term “selectively collapse” can refer to collapsing at least a portion of one GVPS type in a greater amount that any other GVPS type in a mixture containing a plurality of GVPS types.

The second US pulse(s) can comprise a pressure value can be selected from the first collapse pressure profile (e.g., peak acoustic pressure) that optimally maximizes collapse of the first gas vesicles while minimizing collapse of the secondary gas vesicles. The first US pulse(s) and the second US pulse(s) can comprise a pressure value lower than the second selectable collapse pressure value. The method can comprise: applying fifth US pulse(s) (e.g., collapsing ultrasound for the second gas vesicles) to the same or different target site of the subject. The fifth US pulse(s) (e.g., collapsing ultrasound for the second gas vesicles) can comprise a pressure value equal to or higher than the second selectable collapse pressure value. In some embodiments, the fifth US pulse(s) (e.g., collapsing ultrasound for the second gas vesicles) induces secondary gas vesicle collapse. In some embodiments, the fifth US pulse(s) increases the diffusivity of the hydrogel material by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values). In some embodiments, the fifth US pulse(s) increases the porosity of the hydrogel composition by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values). The hydrogel composition can comprise: a plurality of secondary payload molecules. A secondary payload molecule can comprise a hydrodynamic radius at least 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values) larger than a payload molecule.

In some embodiments, the fifth US pulse(s) increases the rate of secondary payload molecule release from the hydrogel composition by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values).

The method can comprise: confirming the delivery of the secondary payload molecules to the same or different target site of the subject. Confirming the delivery of the secondary payload can comprise detecting reduced scattering of the fifth US pulse(s) by the secondary gas vesicles. The secondary gas vesicles can be capable of acting as a contrast agent at the fourth US pulse(s) and sixth US pulse(s) but not at the fifth US pulse(s). Less than about 40%, 30%, 20%, 10%, 5%, 3%, 1%, 0.1%, or a number or a range between any of these values, of the secondary gas vesicles can be not collapsed following the second US pulse(s).

Applying an US pulse(s) can comprise applying focused US pulse(s). Applying an US pulse(s) can comprise applying ultrasound at a frequency of 100 kHz to 100 MHz. Applying an US pulse(s) can comprise applying ultrasound at a frequency of 0.2 to 1.5 MHz. Applying an US pulse(s) can comprise applying ultrasound having a mechanical index in a range between 0.2 and 0.6. The first US pulse(s), second US pulse(s), third US pulse(s), fourth US pulse(s), fifth US pulse(s), and/or sixth US pulse(s) can comprise a peak pressure of about 40 kPa to about 800 kPa. The first US pulse(s), second US pulse(s), third US pulse(s), fourth US pulse(s), fifth US pulse(s), and/or sixth US pulse(s) can comprise a peak pressure of about 70 kPa to about 150 kPa, and/or about 440 kPa to about 605 kPa. The method can comprise applying imaging ultrasound for the secondary gas vesicles prior to applying collapsing ultrasound for the secondary gas vesicles. In some embodiments, the peak pressure is peak positive pressure. In some embodiments, the peak pressure is peak negative pressure. In some embodiments, applying first US pulse(s) comprises applying a single first US pulse, applying second US pulse(s) comprises applying a single second US pulse, applying third US pulse(s) comprises applying a single third US pulse, applying fourth US pulse(s) comprises applying a single fourth US pulse, applying fifth US pulse(s) comprises applying a single fifth US pulse, and/or applying sixth US pulse(s) comprises applying a single sixth US pulse. In some embodiments, applying first US pulse(s) comprises applying a set of first US pulses, applying second US pulse(s) comprises applying a set of second US pulses, applying third US pulse(s) comprises applying a set of third US pulses, and/or applying fourth US pulse(s) comprises applying a set of fourth US pulses, applying fifth US pulse(s) comprises applying a set of fifth US pulses, and/or applying sixth US pulse(s) comprises applying a set of sixth US pulses. In some embodiments, first US pulse(s) comprises a set of first US pulses, second US pulse(s) comprises a set of second US pulses, third US pulse(s) comprises a set of third US pulses, fourth US pulse(s) comprises a set of fourth US pulses, fifth US pulse(s) comprises a set of fifth US pulses, and/or sixth US pulse(s) comprises a set of sixth US pulses. In some embodiments, first US pulse(s) comprises a single first US pulse, second US pulse(s) comprises a single second US pulse, third US pulse(s) comprises a single third US pulse, fourth US pulse(s) comprises a single fourth US pulse, fifth US pulse(s) comprises a single fifth US pulse, and/or sixth US pulse(s) comprises a single sixth US pulse. The peak acoustic pressure for a single US pulse or a set of US pulses can range from about 1 pascals to about 10 megapascals (and all combinations and subcombinations of ranges therein). In some embodiments, the pulse(s) comprise a pulse train, and can have a pulse duration ranging from about 10 microseconds to about 10 seconds (and all combinations and subcombinations of ranges therein). In some embodiments, applying pulse(s) comprises applying multiple pulses to ensure spatial coverage and/or complete GV collapse.

In certain embodiments, the method includes applying a set of imaging pulses from an ultrasound transmitter to the target site, and receiving ultrasound signal at a receiver. In certain instances, the ultrasound signal detected by the receiver includes an ultrasound echo signal. Additional information of ultrasound systems and methods can be found in related publications as will be understood by a person skilled in the art.

Methods for performing ultrasound imaging are known in the art and can be employed in methods of the current disclosure. In certain aspects, an ultrasound transducer, which comprises piezoelectric elements, transmits an ultrasound imaging signal (or pulse) in the direction of the target site. Variations in the acoustic impedance (or echogenicity) along the path of the ultrasound imaging signal causes backscatter (or echo) of the imaging signal, which is received by the piezoelectric elements. The received echo signal is digitized into ultrasound data and displayed as an ultrasound image. Conventional ultrasound imaging systems comprise an array of ultrasonic transducer elements that are used to transmit an ultrasound beam, or a composite of ultrasonic imaging signals that form a scan line. The ultrasound beam is focused onto a target site by adjusting the relative phase and amplitudes of the imaging signals. The imaging signals are reflected back from the target site and received at the transducer elements. The voltages produced at the receiving transducer elements are summed so that the net signal is indicative of the ultrasound energy reflected from a single focal point in the subject. An ultrasound image is then composed of multiple image scan lines.

In some embodiments, imaging the target site is performed by applying or transmitting an imaging ultrasound signal from an ultrasound transmitter to the target site and receiving a set of ultrasound data at a receiver. The ultrasound data can be obtained using a standard ultrasound device, or can be obtained using an ultrasound device configured to specifically detect the contrast agent used. Obtaining the ultrasound data can include detecting the ultrasound signal with an ultrasound detector. In some embodiments, the imaging step further comprises analyzing the set of ultrasound data to produce an ultrasound image.

In certain embodiments, the ultrasound signal has a transmit frequency of at least 1 MHz, 5 MHz, 10 MHz, 20 MHz, 30 MHz, 40 MHz or 50 MHz. For example, an ultrasound data is obtained by applying to the target site an ultrasound signal at a transmit frequency from 4 to 11 MHz, or at a transmit frequency from 14 to 22 MHz. In some instances, the imaging frequency is selected so as to maximize the contrast generated by the administered contrast agent.

In the embodiments herein described, the collapsing ultrasound and imaging ultrasound are selected to have a collapsing pressure and an imaging pressure amplitude based on the acoustic collapse pressure profile (e.g., peak acoustic pressure) of the GVPS type used in the hydrogel composition. In some instances, the ultrasound pressure, including the collapsing ultrasound pressure and the imaging ultrasound pressure can be referred to as the “peak positive pressure” of the ultrasound pulses. The term “peak positive pressure” can refer to the maximum pressure amplitude of the positive pulse of a pressure wave, typically in terms of the difference between the peak pressure and the ambient pressure at the location in the person or specimen that is being imaged.

The target site can comprise a site of disease or disorder or can be proximate to a site of a disease or disorder. The location of the one or more sites of a disease or disorder can be predetermined. The location of the one or more sites of a disease or disorder can be determined during the method (e.g., by an imaging-based method). The target site can comprise a tissue, such as, for example, adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue and/or fat tissue. The tissue can be inflamed tissue. The tissue can comprise (i) grade I, grade II, grade III or grade IV cancerous tissue; (ii) metastatic cancerous tissue; (iii) mixed grade cancerous tissue; (iv) a sub-grade cancerous tissue; (v) healthy or normal tissue; and/or (vi) cancerous or abnormal tissue. In some embodiments, upon administration, the hydrogel composition accumulates in vasculature of cancerous tissue. Exemplary target sites include collections of microorganisms, including, bacteria or archaea in a solution in vitro, as well as cells grown in an in vitro culture, including, primary mammalian, cells, immortalized cell lines, tumor cells, stem cells, and the like. Additional exemplary target sites include tissues and organs in an ex vivo colture and tissue, organs, or organs systems in a subject, for example, lungs, brain, kidney, liver, heart, the central nervous system, the peripheral nervous system, the gastrointestinal system, the circulatory system, the immune system, the skeletal system, the sensory system, within a body of an individual and additional environments identifiable by a skilled person. The term “individual” or “subject” or “patient” as used herein in the context of imaging includes a single plant or animal and in particular higher plants or animals and in particular vertebrates such as mammals and more particularly human beings. Types of ultrasound imaging of biological target sites include abdominal ultrasound, vascular ultrasound, obstetrical ultrasound, hysterosonography, pelvic ultrasound, renal ultrasound, thyroid ultrasound, testicular ultrasound, and pediatric ultrasound as well as additional ultrasound imaging as would be understood by a skilled person.

In some embodiments, the subject has a disease or disorder. The subject can be a mammal (e.g., a marine mammal). In some embodiments, the subject has a disease of the GI tract. In some embodiments, the disease of the GI tract can be an inflammatory bowel disease, such as, for example, Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease or Behcet's disease. The target site can comprise a section or subsection of the GI tract, such as, for example, the stomach, proximal duodenum, distal duodenum, proximal jejunum, distal jejunum, proximal ileum, distal ileum, proximal cecum, distal cecum, proximal ascending colon, distal ascending colon, proximal transverse colon, distal transverse colon, proximal descending colon and distal descending colon, or any combination thereof.

A subject can have, or be suspected of having, a diseases or disorder. Various diseases and disorders can be treated with the hydrogel compositions provided herein. Diseases and disorders disclosed herein include, but are not limited to, rheumatoid arthritis, osteoarthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia greata, seronegative arthropathy, arthropathy, Reiter's disease, psoriatic arthropathy, ulcerative colitic arthropathy, enteropathic synovitis, chlamydia, yersinia and salmonella associated arthropathy, spondyloarthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, acquired immunodeficiency disease syndrome, acquired immunodeficiency related diseases, hepatitis B, hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylo sing spondylitis associated lung disease, vasculitic diffuse lung disease, haemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjörgren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo acute liver disease, chronic liver diseases, alcoholic cirrhosis, alcohol-induced liver injury, cholestasis, idiosyncratic liver disease, Drug-Induced hepatitis, Non-alcoholic Steatohepatitis, allergy and asthma, group B streptococci (GBS) infection, mental disorders, depression, schizophrenia, Th2 Type and Th1 Type mediated diseases, acute pain, chronic pain, cancer, lung cancer, breast cancer, stomach cancer, bladder cancer, colon cancer, pancreatic cancer, ovarian cancer, prostate cancer, rectal cancer, hematopoietic malignancies, leukemia, lymphoma, abetalipoprotemia, acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti cd3 therapy, antiphospholipid syndrome, anti-receptor hypersensitivity reactions, aortic and peripheral aneuryisms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chronic myelocytic leukemia (CMIL), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, corpulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatitis, dermatologic conditions, diabetes, diabetes mellitus, diabetic aterio sclerotic disease, diffuse Lewy body disease, dilated congestive cardiomyopathy, disorders of the basal ganglia, Down's syndrome in middle age, drug-induced movement disorders induced by drugs which block CNS dopamine receptors, drug sensitivity, eczema, encephalomyelitis, endocarditis, endocrinopathy, epiglottitis, epstein-barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallervorden-Spatz disease, Hashimoto's thyroiditis, hay fever, heart transplant rejection, hemachromatosis, hemodialysis, hemolytic uremic syndrome/thrombolytic thrombocytopenic purpura, hemorrhage, hepatitis A, H is bundle arrythmias, HIV infection/HIV neuropathy, Hodgkin's disease, hyperkinetic movement disorders, hypersensitivity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, asthenia, infantile spinal muscular atrophy, inflammation of the aorta, influenza A, ionizing radiation exposure, iridocyclitis/uveitis/optic neuritis, ischemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphedema, malaria, malignant lymphoma, malignant histiocytosis, malignant melanoma, meningitis, meningococcemia, metabolic/idiopathic, migraine headache, mitochondrial multi. system disorder, mixed connective tissue disease, monoclonal gammopathy, multiple myeloma, multiple systems degenerations, Mencel Dejerine-Thomas Shi-Drager degeneration, Machado-Joseph degeneration, myasthenia gravis, Mycobacterium avium intracellulare, Mycobacterium tuberculosis, myelodyplastic syndrome, myocardial infarction, myocardial ischemic disorders, nasopharyngeal carcinoma, neonatal chronic lung disease, nephritis, nephrosis, neurodegenerative diseases, neurogenic muscular atrophies, neutropenic fever, non-hodgkins lymphoma, occlusion of the abdominal aorta and its branches, occulsive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral arteriosclerotic disease, peripheral vascular disorders, peritonitis, pernicious anemia, Pneumocystis carinii pneumonia, pneumonia, POEMS syndrome, polyneuropathy, organomegaly, endocrinopathy, monoclonal gammopathy, skin changes syndrome, post perfusion syndrome, post pump syndrome, post-MI cardiotomy syndrome, preeclampsia, progressive supranuclear palsy, primary pulmonary hypertension, radiation therapy, Raynaud's phenomenon and disease, Raynoud's disease, Refsum's disease, regular narrow QRS tachycardia, renovascular hypertension, reperfusion injury, restrictive cardiomyopathy, sarcomas, scleroderma, senile chorea, senile dementia of Lewy body type, seronegative arthropathies, shock, sickle cell anemia, skin allograft rejection, skin changes syndrome, small bowel transplant rejection, solid tumors, specific arrythmias, spinal ataxia, spinocerebellar degenerations, streptococcal myositis, structural lesions of the cerebellum, subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, telangiectasia, thromboangitis obliterans, thrombocytopenia, toxicity, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, viral encephalitis/aseptic meningitis, viral-associated hemaphagocytic syndrome, Wernicke-Korsakoff syndrome, Wilson's disease, xenograft rejection of any organ or tissue, acute coronary syndromes, acute idiopathic polyneuritis, acute inflammatory demyelinating polyradiculoneuropathy, acute ischemia, adult Still's disease, alopecia greata, anaphylaxis, anti-phospholipid antibody syndrome, aplastic anemia, arteriosclerosis, atopic eczema, atopic dermatitis, autoimmune dermatitis, autoimmune disorder associated with Streptococcus infection, autoimmune enteropathy, autoimmune hearing loss, autoimmune lymphoproliferative syndrome (ALPS), autoimmune myocarditis, autoimmune premature ovarian failure, blepharitis, bronchiectasis, bullous pemphigoid, cardiovascular disease, catastrophic antiphospholipid syndrome, celiac disease, cervical spondylosis, chronic ischemia, cicatricial pemphigoid, clinically isolated syndrome (cis) with risk for multiple sclerosis, conjunctivitis, childhood onset psychiatric disorder, chronic obstructive pulmonary disease (COPD), dacryocystitis, dermatomyositis, diabetic retinopathy, diabetes mellitus, disk herniation, disk prolapse, drug induced immune hemolytic anemia, endocarditis, endometriosis, endophthalmitis, episcleritis, erythema multiforme, erythema multiforme major, gestational pemphigoid, Guillain-Barre syndrome (GBS), hay fever, Hughes syndrome, idiopathic Parkinson's disease, idiopathic interstitial pneumonia, IgE-mediated allergy, immune hemolytic anemia, inclusion body myositis, infectious ocular inflammatory disease, inflammatory demyelinating disease, inflammatory heart disease, inflammatory kidney disease, IPF/UIP, iritis, keratitis, keratoconjunctivitis sicca, Kussmaul disease or Kussmaul-Meier disease, Landry's paralysis, Langerhan's cell histiocytosis, livedo reticularis, macular degeneration, microscopic polyangiitis, morbus bechterev, motor neuron disorders, mucous membrane pemphigoid, multiple organ failure, myasthenia gravis, myelodysplastic syndrome, myocarditis, nerve root disorders, neuropathy, non-A non-B hepatitis, optic neuritis, osteolysis, pauciarticular JRA, peripheral artery occlusive disease (PAOD), peripheral vascular disease (PVD), peripheral artery, disease (PAD), phlebitis, polyarteritis nodosa, periarteritis nodosa, polychondritis, polymyalgia rheumatica, poliosis, polyarticular JRA, polyendocrine deficiency syndrome, polymyositis, polymyalgia rheumatica (PMR), post-pump syndrome, primary Parkinsonism, prostatitis, pure red cell aplasia, primary adrenal insufficiency, recurrent neuromyelitis optica, restenosis, rheumatic heart disease, sapho (synovitis, acne, pustulosis, hyperostosis, and osteitis), scleroderma, secondary amyloidosis, shock lung, scleritis, sciatica, secondary adrenal insufficiency, silicone associated connective tissue disease, sneddon-wilkinson dermatosis, spondilitis ankylosans, Stevens-Johnson syndrome (SJS), systemic inflammatory response syndrome, temporal arteritis, toxoplasmic retinitis, toxic epidermal necrolysis, transverse myelitis, TRAPS (tumor necrosis factor receptor, type 1 allergic reaction, type II diabetes, urticaria, usual interstitial pneumonia (UIP), vasculitis, vernal conjunctivitis, viral retinitis, Vogt-Koyanagi-Harada syndrome (VKH syndrome), wet macular degeneration, wound healing, Yersinia, and Salmonella associated arthropathy.

### Pharmaceutically Acceptable Compositions and Methods of Administration

Administering can comprise aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, and/or intradermal injection, or any combination thereof.

The period of time between the administering and applying the first US pulse(s) can be about 100 hours, 50 hours, about 48 hours, about 44 hours, about 40 hours, about 35 hours, about 30 hours, about 24 hours, 8 hours, about 7 hours, about 6 hours, about 5 hours, about 4 hours, about 3 hours, about 2 hours, about 1 hour, about 30 minutes, about 15 minutes, about 10 minutes, about 5 minutes, about 1 minute, or a number or a range between any two of these values.

In methods herein described, administering the hydrogel composition can be performed in any way suitable to deliver a hydrogel composition to the target site for payload delivery. In some embodiments, the hydrogel composition can be administered to the target site locally or systemically.

The wording “local administration” or “topic administration” as used herein indicates any route of administration by which a hydrogel composition is brought in contact with the body of the individual, so that the resulting hydrogel composition location in the body is topic (limited to a specific tissue, organ or other body part where the imaging is desired). Exemplary local administration routes include injection into a particular tissue by a needle, gavage into the gastrointestinal tract, and spreading a solution containing hydrogel composition on a skin surface.

The wording “systemic administration” as used herein indicates any route of administration by which a hydrogel composition is brought in contact with the body of the individual, so that the resulting hydrogel composition location in the body is systemic (i.e. non limited to a specific tissue, organ or other body part where the imaging is desired). Systemic administration includes enteral and parenteral administration. Enteral administration is a systemic route of administration where the substance is given via the digestive tract, and includes but is not limited to oral administration, administration by gastric feeding tube, administration by duodenal feeding tube, gastrostomy, enteral nutrition, and rectal administration. Parenteral administration is a systemic route of administration where the substance is given by route other than the digestive tract and includes but is not limited to intravenous administration, intra-arterial administration, intramuscular administration, subcutaneous administration, intradermal, administration, intraperitoneal administration, and intravesical infusion.

Accordingly, in some embodiments of methods herein described, administering a contrast agent can be performed topically or systemically by intradermal, intramuscular, intraperitoneal, intravenous, subcutaneous, intranasal, rectal, vaginal, and oral routes. In particular, a hydrogel composition can be administered by infusion or bolus injection, by absorption through epithelial or mucocutaneous linings (e.g., oral mucosa, vaginal, rectal and intestinal mucosa, etc.) and can optionally be administered together with other biologically active agents. In some embodiments of methods herein described, administering a hydrogel composition can be performed by injecting the hydrogel composition into a subject at the target site of interest, such as in a body cavity or lumen. In some embodiments, it can be performed by spreading a solution containing the hydrogel composition on a region of the skin.

The hydrogel composition can be formulated for oral administration. The hydrogel composition can comprise an enteric coating. The enteric coating can comprise one or more of acetyltributyl citrate, carbomers, cellulose acetate phthalate, cellulose acetate succinate, ethyl cellulose, guar gum, hypromellose acetate succinate, hypromellose phthalate, polymethacrylates, polyvinyl acetate phthalate, shellac, tributyl citrate, triethyl citrate, white wax, and zein. In some embodiments, the method does not comprise a pH-dependent payload release mechanism.

In another aspect, the present invention provides pharmaceutically acceptable compositions which comprise a therapeutically-effective amount of a hydrogel composition disclosed herein. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension: (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the hydrogel composition. The pharmaceutical compositions can comprise one or more pharmaceutically-acceptable carriers.

The phrase “therapeutically-effective amount” as used herein means that amount of hydrogel composition disclosed herein which is effective for producing some desired therapeutic effect, e.g., cancer treatment, at a reasonable benefit/risk ratio.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth: (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (1) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the hydrogel composition which produces a therapeutic effect. Generally, out of one hundred percent, this amount will range from about 1% to about 99% of active ingredient, preferably from about 5% to about 70%, most preferably from about 10% to about 30%.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a respiration uncoupling agent as an active ingredient. A hydrogel composition may also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more respiration uncoupling agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate.

Dosage forms for the topical or transdermal administration of hydrogel compositions include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain, in addition to a respiration uncoupling agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Ophthalmic formulations, eye ointments, powders, solutions and the like, are also contemplated as being within the scope of this invention.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Actual dosage levels of the active ingredients in the pharmaceutical compositions of this invention may be determined by the methods of the present invention so as to obtain an amount of the active ingredient, which is effective to achieve the desired therapeutic response for a particular subject, composition, and mode of administration, without being toxic to the subject.

Also provided herein are kits comprising one or more compositions described herein, in suitable packaging, and may further comprise written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. A kit may comprise one or more unit doses described herein. The compositions can be in the form of kits of parts. In a kit of parts, one or more components of the compositions disclosed herein are provided independent of one another (e.g., the polymers, payload, and/or GVs are provided as separate compositions) and are then employed (e.g., by a user) to generate the compositions.

### EXAMPLES

Some aspects of the embodiments discussed above are disclosed in further detail in the following examples, which are not in any way intended to limit the scope of the present disclosure.

### Methods of the Examples

GV-Gel Preparation

50 μl of hydrogel reaction mixture is made by copolymerizing a predetermined volume fraction of acrylamide and bis-acrylamide (A7168: Sigma Aldrich) in prefiltered pH 7.4 phosphate buffered saline (PBS) solution at a fixed cross-linking density of 2.7%. 50 μl of purified Anabaena Flos-Aquae GVs solution obtained following the protocol of Lakshman et al (2017) was concentrated to a predetermined volume fraction (OD 24=1% vol. GV) and added to the hydrogel reaction mixture. The hydrogel reaction mixture is then degassed in a vacuum chamber for 30 min to rid the system of oxygen. 0.1 and 0.5 μl of initiators tetramethyl ethylene diamine (T9281: Sigma Aldrich) and 10% ammonium persulfate solution (A3678: Sigma Aldrich) respectively was then added to the reaction mixture and agitated slightly using a pipette to start the free-radical polymerization at room temperature (25C). Using the prepared syringe-capillary system, the hydrogel reaction mixture is immediately withdrawn into the glass capillary, leaving about a 1 inch of the glass capillary empty at the top for dye loading later on. The bottom of the glass capillary is quickly capped with some clay to prevent the hydrogel from drying or leaking out. To collapse the GVs in the GV-gel, the filled glass capillary is first placed into a PBS-filled container and held in place by solidified 1% Agarose. An L11-4V 128-element linear array ultrasound transducer (Verasonics) operating at 45 V was then held in place over the capillary for 3 mins to collapse the GVs in the gel. GV collapse is verified visually from the loss of gel opacity, and from the loss of ultrasound signal within the gel. These capillary loaded gels are then equilibrated at room temperature with PBS for at least 6 hours prior to experiments.

BSA Diffusivity Experiment

Albumin from Bovine Serum (BSA) with AlexaFluor™ 647 conjugate (A34785: ThermoFisher Scientific) was diluted to 500 μg/ml using PBS and equilibrated at room temperature for 5 mins. A 1 ml BD Luer-Lok™ tip syringe fitted with a blunt 30G needle (SAI Infusion Technologies) was then used to inject the BSA-Alexa Fluor dye near the interface of the hydrogel casted within a 3-inch open-ended glass capillary (1B120-3: World Precision Instruments), ensuring all the air bubbles are extracted prior to experiments. The experiments were then carried out following the protocols of Hettiarachi et al (2018) for rapid measurements of protein diffusion through hydrogels using a Zeiss LSM 800 inverted microscope fitted to a 2.5× objective lens (numerical aperture: 0.075). Fluorescence imaging was performed under an Alexa 647 channel (excitation: 587/25 nm, emission: 647/70 nm) and images were taken every 6 mins over a period of 2h.

Calculation of Diffusion Coefficient

The time series of fluorescence images from the capillary diffusivity experiments capillary were imported into MATLAB for image processing. The time evolution of the fluorescence intensity across the capillary length from the fluid-gel interface was recorded and averaged across the capillary cross-sectional area. Since the length of the gel (5 cm) is significantly larger that its diameter (0.6 mm) one can approximate the protein-dye transport in the gel to follow a 1-D diffusion model through an infinite slab given by the following equation:

$$F ( x , t ) = K erfc ( \frac{x}{2 \sqrt{{D}_{eff} t}} ) \qquad ( 1 )$$

where K and D_{eff} are constants that represent the gel partitioning and diffusion coefficients of BSA respectively, F is the fluorescence intensity normalized to the dye reservoir intensity, x is the distance from the interface and t is the time. The experimental data was then fitted to equation (1) using a nonlinear programming solver on MATLAB to obtain the two constants.

GV-Gel Cargo Preparation for In Vitro Release

Preparation of the GV-gel cargoes utilized the same hydrogel recipe as described in the BSA diffusivity experiments with BSA-AlexaFluor at a final dye concentration of 500 μg/ml also dissolved in the polymerization media. The reaction mixture was then casted into 8 μl cubic moulds as shown in FIG. 3A. Collapse of GVs in the gel also utilize a similar protocol as that described in the GV-gel preparation section, fixing instead the cargo-containing moulds into the solidified 1% Agarose.

In Vitro Cargo Release Experiment

Ten pieces of the cubic GV-gel cargoes were suspended in 30 ml fresh PBS and stirred at 37° C. and 100 rpm. The experiment was performed in a dark room to minimize fluorophore photobleaching. Every 2 hours for a total of 12 hours, 10 μL of the suspension was aliquoted into a 90-well plate containing 100 μL of PBS. The wells were then analysed using a Spectramax M5 plate reader with the following wavelength settings: excitation: 587/25 nm and emission: 647/70 nm. After 72 hours, the suspension was aliquoted again to determine the total protein-dye payload present in all ten GV-gel cargoes. Fluorescence intensities collected during the time series were then normalised to the final intensity at 72 hours to determine the release kinetics of the GV-gel cargoes.

### Example 1

### Demonstration of Ultrasound-Modulated Diffusion Using GV-Gels

The ability of embedded GVs to affect diffusion properties through a material was tested with polyacrylamide gel used as a proxy for a generic polymer system. Adapting the rapid diffusivity quantification protocols devised by Hettiarachi et al (2018), GV-gels of varying GV and monomer/cross-linker volume fractions were casted inside glass capillaries loaded with a fluorescent BSA-AlexaFluor 647 reservoir. Bovine serum albumin (BSA) can be employed as a model drug payload. The evolution of fluorescence through the length of a GV-gel was recorded as a time-series under a confocal microscope using the set-up in FIG. 2A, with representative fluorescence intensity curves shown in FIG. 2B.

As shown in FIG. 2C, the presence of intact GVs hinder GV diffusion through the gel with increasing effect as more GVs are dispersed throughout the hydrogel matrix. Hydrogels with GVs collapsed in situ using ultrasound however, enabled faster BSA diffusion through the gel. Ultrasound can applied using a diagnostic ultrasound transducer operating at 6 MHz. These two effects in combination can enable a user to create triggered drug delivery vehicles by modulating the material diffusive properties at a molecular level using ultrasound.

GVs can also be preclustered to larger fractal dimensions of around 1 m using a biotin-streptavidin reaction prior to gel immobilization. For the same polyacrylamide content of 10%, a 100-fold change in BSA diffusivity through the gel upon collapse of these GV clusters was observed (FIG. 2D). This result alludes to the formation of closely linked voids—or percolation channels—within the gel since clustered GVs are more closely spaced than their unclustered counterparts in the hydrogel matrix due to the biotin-streptavidin binding.

### Example 2

### Demonstration of Ultrasound-Modulated Control of In Vitro Payload Release Kinetics Using GV-Gels

An in vitro GV-gel cargo release experiment was designed (FIGS. 3A-3B) to verify that the earlier changes in diffusivity observed on a molecular level translate to differences in payload release kinetics on the macroscopic-length scales of drug delivery cargoes. As shown in FIG. 3C, GV-gels exposed to ultrasound are able to release their payload at a much faster rate than the polyacrylamide gel negative control. Much slower release kinetics were also observed as predicted for the gels with intact GV-gels that have not yet been exposed to ultrasound. This result was also demonstrated in clustered GV-gels (FIG. 3D), showing much more significant changes in payload release kinetics compared to the negative control before and after ultrasound-induced GV collapse. These sets of experiment therefore validate the GV nanoadditives disclosed herein as agents that can easily couple material diffusive properties to ultrasound in a controllable manner.

### Example 3

### GV-Gels Made from Different Polymers Produce Ultrasound Modulated Diffusion Changes Across Payloads of Different Length Scales

Hydrogels of varying gas vesicle (GV) and monomer volume fractions were casted inside glass capillaries and loaded with a fluorescent reservoir on one end. Using the rapid diffusivity quantification protocols devised by Hettiarachi et al (2018), the evolution of fluorescence through the length of a GV-gel was recorded as a time-series under a confocal microscope using the set-up depicted in FIG. 2A. The fluorescence intensity profiles along the length of the hydrogel was fitted to the 1-D diffusion equation solution to determine the diffusion coefficient.

Discrete GVs Embedded in Polyacrylamide Hydrogel Produce Fold Changes in Material Diffusivity to BSA Payload when Exposed to Ultrasound

GV-gels were made by dispersing purified gas vesicles obtained from Anabaena flos-aquae in the hydrogel reaction mixture containing 2-10% volume fraction of 19:1 acrylamide/bisacrylamide solution and trace amounts of tetramethyl ethylene diamine (TEMED) and ammonium persulfate (APS) initiators. The reservoir contained phosphate buffered saline (PBS) and bovine serum albumin (BSA) labelled with the Alexa 647 fluorophore.

The presence of intact GVs was found to obstruct BSA diffusion in the gel with increasing effect as more GVs are dispersed throughout the hydrogel matrix (FIG. 4A). This was expected since solute transport within hydrogels occur primarily within the water-filled regions, which have been reduced as a result of GV addition. Upon in situ collapse of the embedded GVs using a Verasonics L10 diagnostic transducer operating with a parabolic B-mode pulse sequence at 6 MHz and 40V, the diffusivity values were observed to return to comparable magnitudes of the gel-only negative controls as a result of the excluded GV volume within the gel being liberated. This ultrasound-modulated diffusivity change was only observable in gels that contained GVs (FIG. 4A). The dynamic range between the intact and collapsed GV cases also widened at higher concentrations of GV and polymer. (FIG. 4A). Thus, the hydrogel compositions and methods provided herein were able to achieve an 8-fold change in material diffusivity by using ultrasound to influence the material properties at a molecular level, demonstrating proof of principle for the hydrogel compositions and methods provided herein.

Clustered GVs Embedded in Polyacrylamide Hydrogel Produce Greater Fold Changes in Material Diffusivity to BSA Payload when Exposed to Ultrasound

Another strategy to influence solute transport and enhance the dynamic range of the disclosed platform methods and compositions is to precluster GVs using a biotin-streptavidin reaction (FIG. 4B) to larger fractal dimensions of around 1 μm prior to gel immobilization. As shown in FIG. 4C, this change in geometry resulted in a much larger diffusion fold change compared to that of unclustered GV-gels. Without being limited by any particular theory, this is presumed to be a result of the collapse of larger GV clusters that generate more interconnected voids to facilitate rapid drug release.

Clustered GV-Gels Produce Ultrasound Induced Material Diffusivity Changes for a Variety of Payload and Hydrogel Length Scales

As proof of principle for the hydrogel compositions and methods provided herein, the versatility of exploiting the GV US-induced collapse phenomenon to trigger changes in material diffusivity was demonstrated (FIG. 4D) in a variety of payloads and hydrogel systems across clinically-relevant length scales (Tables 1-2).

TABLE 1
List of hydrogel systems characterized

| Hydrogel | Formation | Pore Size (nm) |
|---|---|---|
| 2-10% | Chemical - Free Radical | 10-20 |
| Polyacrylamide | | |
| 10% Alginate | Physical - Ionic Interaction | 5 |
| 10% Agarose | Physical - Hydrogen Bonding | 100 |

TABLE 2
List of payloads added to the reservoir

| | Stokes' | |
|---|---|---|
| Payload | Diameter (nm) | Clinical Relevance |
| Alexa 647 fluorophore | <1 | Small molecules (e.g. NSAID) |
| BSA | 5 | Peptides and small proteins |
| Adalimumab | 10 | Antibody therapies |
| Quantum Dot | 25 | AAV |

### Example 4

### In Vitro Release of Payload from GV-Gel Vehicle

GV-gel tablets were formed by setting the polymer mixture, clustered GV and the fluorescent BSA-AlexaFluor 647 payload together within 3D-printed moulds (FIG. 3A).

Ultrasound Imaging and Targeting of GV-Gels

Targeting of GV-gels in vitro was performed by casting delivery vehicles into a plate containing 1% agarose (FIG. 5A). Certain GV-gels were picked to be imaged and collapsed (FIG. 5B) using a parabolic B-mode pulse sequence on the Verasonics L22 transducer operating at 18 MHz at 12 V and 20 V respectively. Fluorescence images were taken immediately after GV collapse at t=0 and at t=12 hours and the difference in the intensities between the two timepoints is shown in FIG. 5C. From these experimental results, it is demonstrated that only vehicles in the targeted locations experienced payload release, making it an ideal platform for spatiotemporal targeting.

Ultrasound Enhanced Payload Release Kinetics from GV-Gels

These GV-gel tablets were then placed into a stirred vial filled with simulated stomach fluid (FIG. 5D) for 12 hours. Fluorescence intensity of the stomach fluid was then tracked over time as an indication of drug release kinetics. Consistent with the findings discussed above, FIG. 5E shows that macroscopic GV-gels exposed to ultrasound are also capable of releasing their payload at a much faster rate than the gel-only negative control. To further reduce the leak observed, these GV-gels were preincubated for 6 hours in PBS before determine drug release kinetics. This step resulted in the significant reduction of leakiness to a low level of 60.1% (N=3), which could be due to the release of the more loosely bound protein payload located closer to the surface of the GV-gel tablets.

### Example 5

### In Vivo Release of Payload from GV-Gel Vehicle in Mice

An in vivo proof of principle experiment for the hydrogel compositions and methods provided herein was performed as follows. Six 10-week old male C57BL/6J mice were fasted 16 hours prior to being given enemas according to the groups they were in: (i) two mice received a 10 mg dose of GV-gels suspended in PBS followed by ultrasound imaging and collapse (ii) two mice received a 10 mg dose of GV-gels suspended in PBS but without ultrasound treatment and (iii) two mice received a 10 mg dose of polyacrylamide gels (no GV) with ultrasound imaging and collapse. The gels were prepared according to the procedures detailed out in Example 4.

In Vivo Imaging of GV-Gels

Ultrasound imaging and collapse was performed using a parabolic B-mode pulse sequence on the Verasonics L22 transducer operating at 18 MHz at 12 V and 25 V respectively. Representative pre and post-collapse x-AM images of the vehicles in vivo are shown in FIG. 6A. Gels containing gas vesicles were found to display a faint x-AM signal at 12 V which was partially eliminated upon ramping up the voltage to 25V to trigger partial collapse of the gas vesicle population in the gels, thus triggering payload release. Gels that did not contain gas vesicles did not show up under x-AM imaging.

In Vivo Release of Payload from GV-Gels Using Ultrasound

After enema administration, the mice were sacrificed by cervical dislocation following anaesthesia, and the abdominal cavity was subsequently dissected with the entirety of the lower gastrointestinal tract isolated and stored in a petri dish containing PBS at 4° C. The plates were then fluorescently imaged. Twelve hours later, the intestines were gently flushed with PBS to remove the GV-gels and then fluorescently imaged. Representative images of the three different mice groups are shown in FIG. 6B. It was found that after 12 hours, there remains a clear fluorescence signal localized within the mice large intestine only for the case where ultrasound was used to collapse gels that contained GVs. This can be explained by the fact that most fluorescently labelled BSA payload released from the gel within the 12-hour period would have stuck to the viscous mucosal lining on the lumen instead of being flushed out with the gel like those in the negative control mice groups (ii) and (iii).

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A hydrogel composition, comprising:
a polymer scaffold comprising a plurality of polymers,
a plurality of payload molecules, and
a plurality of gas vesicles.

2. The hydrogel composition of claim 1, wherein the hydrogel composition comprises one or more particles.

3. The hydrogel composition of claim 1, wherein the plurality of payload molecules and the plurality of gas vesicles are embedded within the polymer scaffold.

4. The hydrogel composition of claim 1, wherein the plurality of polymers are cross-linked via (1) covalent bonds and/or non-covalent bonds, (2) inter-polymer bonds and/or intra-polymer bonds, and/or (3) physical crosslinks and/or chemical crosslinks.

5. (canceled)

6. The hydrogel composition of claim 1, wherein the plurality of polymers comprise a copolymer, a natural polymer, a synthetic polymer, a block copolymer, or any combination thereof.

7.-13. (canceled)

14. The hydrogel composition of claim 1, wherein less than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the plurality of payload molecules and/or the plurality of gas vesicles within the hydrogel composition are released from the hydrogel composition within a 24 hour period in the absence of exposure to ultrasound.

15. The hydrogel composition of claim 1, wherein the polymer scaffold comprises a plurality of pores, and wherein the polymer scaffold comprises a porosity of at least about 10%, at least about 20%, at least about 30%, at least about 40%, at least about 50%, at least about 60%, at least about 70%, at least about 80%, or at least about 90%.

16.-18. (canceled)

19. The hydrogel composition of claim 1, wherein the porosity of the polymer scaffold is configured to retain embedded payload molecules with a hydrodynamic radius of less than about 100 nm, about 80 nm, about 60 nm, about 40 nm, about 30 nm, about 20 nm, about 10 nm, about 5 nm, or about 1 nm.

20. The hydrogel composition of claim 1, wherein the average pore size of the polymer scaffold is smaller than the average diameter of the payload molecules.

21.-28. (canceled)

29. The hydrogel composition of claim 1, wherein the plurality of payload molecules comprise a contrast agent, an imaging agent, and/or a diagnostic agent, a small molecule, a nucleic acid, a cell, a vector, a saccharine, a monosaccharide, a disaccharide, a trisaccharide, an oligosaccharide, a polysaccharide, a peptide, a protein, a peptide analog, a peptidomimetic, an antibody or antigen-binding fragment thereof, an antisense oligonucleotide, an siRNA, an shRNA, a ribozyme, an aptamer, a microRNA, a pre-microRNA, plasmid DNA, modified RNA, or any combination thereof.

30. (canceled)

31. The hydrogel composition of claim 1, wherein the plurality of gas vesicles are bacterially-derived gas vesicles and/or archaea-derived gas vesicles.

32. The hydrogel composition of claim 1, wherein the plurality of gas vesicles are capable of gas vesicle collapse, wherein at least one of the plurality of gas vesicle collapse comprises an at least about 10-fold reduction in volume or at least one of the plurality of gas vesicle collapse comprises an at least about 10-fold reduction in acoustic contrast.

33. The hydrogel composition of claim 1, wherein the plurality of gas vesicles are capable of acting as steric diffusion blockers to the payload molecules, and wherein the gas vesicles are capable of impeding the diffusion of the payload molecules through the polymer scaffold.

34. The hydrogel composition of claim 32, wherein the gas vesicle collapse yields the formation of pores and/or percolation channels within the hydrogel composition, and wherein the dimensions of said pores and/or percolation channels are larger than the dimensions of the payload molecules.

35. The hydrogel composition of claim 1, wherein the gas vesicle collapse (1) increases the diffusivity of the hydrogel material by at least about 2-fold, (2) increases the porosity of the hydrogel composition by at least about 2-fold, and/or (3) increases the rate of payload molecule release from the hydrogel composition by at least about 2-fold.

36. (canceled)

37. The hydrogel composition of claim 1, wherein the plurality of gas vesicles comprise clustered gas vesicles, and wherein the clustered gas vesicles comprise aggregates of two or more gas vesicles bound to each other via one or more clustering moieties.

38.-40. (canceled)

41. The hydrogel composition of claim 1, wherein the plurality of gas vesicles comprises a first collapse pressure profile, wherein the first collapse pressure profile comprises a collapse function from which a gas vesicle collapse amount can be determined for a given pressure value, wherein the first collapse pressure profile comprises a first initial collapse pressure where 5% or lower of the plurality of gas vesicles are collapsed, a first midpoint collapse pressure where 50% of the plurality of gas vesicles are collapsed, a first complete collapse pressure where at least 95% of the plurality of gas vesicles are collapsed, any pressure between the first initial collapse pressure and the first midpoint collapse pressure, and any pressure between the first midpoint collapse pressure and the first complete collapse pressure, and wherein a first selectable collapse pressure is equal to or greater than one or more of the first initial collapse pressure, the first midpoint collapse pressure, and the first complete collapse pressure.

42. (canceled)

43. (canceled)

44. The hydrogel composition of claim 1, wherein the hydrogel composition further comprises a plurality of secondary gas vesicles, and wherein the secondary gas vesicles comprise different mechanical, acoustic, and/or surface properties as compared to the gas vesicles.

45.-51. (canceled)

52. A method for the spatial and temporal delivery of payload molecules to a target site of a subject, comprising:
administering a hydrogel composition to a subject, wherein the hydrogel composition comprises (i) a polymer scaffold comprising a plurality of polymers, (ii) a plurality of payload molecules, and (iii) a plurality of gas vesicles; and
applying one or more ultrasonic (US) pulses to a target site of the subject, wherein the one or more US pulses induce the release of payload molecules from the hydrogel composition, thereby delivering payload molecules to the target site.

53.-58. (canceled)

59. A method of treating a disease or disorder, comprising:
administering a hydrogel composition to a subject suffering from a disease or disorder, wherein the hydrogel composition comprises (i) a polymer scaffold comprising a plurality of polymers, (ii) a plurality of payload molecules, and (iii) a plurality of gas vesicles;
applying first ultrasonic (US) pulse(s) to a target site of the subject;
detecting the presence of the hydrogel composition at the target site; and
applying second US pulse(s) to the target site of the subject, wherein the second US pulse(s) induces the release of payload molecules from the hydrogel composition, thereby delivering payload molecules to the target site and treat the subject of the disease or disorder.

60.-98. (canceled)
